(19) United States
(12) Patent Application Publication
(10) Pub. No.: US 2021/0098341 A1
(43) Pub. Date: Apr. 1, 2021

(54) MICROELECTRODE ARRAY AND METHODS OF FABRICATING SAME

(71) Applicant: Paradromics Inc., Austin, TX (US)

(72) Inventors: Yifan Kong, Austin, TX (US); Kevin Boergens, Austin, TX (US); Matthew Angle, Austin, TX (US); Aleksandar Tadic, Austin, TX (US)

(21) Appl. No.: 17/031,584

(22) Filed: Sep. 24, 2020

Related U.S. Application Data
(60) Provisional application No. 62/908,474, filed on Sep. 30, 2019.
(60) Provisional application No. 62/965,663, filed on Jan. 24, 2020.
(60) Provisional application No. 63/005,116, filed on Apr. 3, 2020.

(51) Int. Cl. H01L 23/48; H01L 23/498; H01L 23/522

(52) U.S. Cl. CPC H01L 23/481; H01L 23/532; H01L 23/5226; H01L 23/49805

(57) ABSTRACT

An implantable device and methods for forming the same are provided. The device may comprise: (a) a substrate comprising a plurality of feedthroughs, wherein the plurality of feedthroughs comprises a first conductive material; and (b) an array of microwires extending from the substrate. The array of microwires may be connected or bonded to the plurality of feedthroughs using a biocompatible solder or braze material or intermediate filler material. The array of microwires may comprise a second conductive material that is different from the first conductive material.

### CROSS-REFERENCE

This application claims priority to U.S. Provisional Application No. 62/908,474 filed Sep. 30, 2019, U.S. Provisional Application No. 62/965,663 filed on Jan. 24, 2020, and U.S. Provisional Application No. 63/005,116 filed Apr. 3, 2020, all of which are incorporated herein by reference in their entirety.

### BACKGROUND

Neural-interface probes may be used to obtain a better understanding of brain functionality, which can lead to improved treatment of certain neurological diseases. Such probes are typically implanted into a brain to record neuronal electrical activity. The neuronal recordings may be analyzed to determine how neural circuits process information at a cellular level. Furthermore, such devices can be used to control neural prosthetics for patients who have lost input or output functionality of their brains.

A neural-interface probe may comprise a high density microelectrode array bonded to a chip. The microelectrode array may comprise a plurality of microwires. The microwires may be rigid, or in some cases flexible. There are challenges associated with fabricating a high density microelectrode array having flexible microwires, and insertion of the tips of those flexible microwires into brain tissue. Those challenges may relate to the machining/cutting/etching of different material types, fixturing during the fabrication process, reducing damage during the fabrication process, hermeticity and sealing, fluid transfer and ion migration, etc.

### SUMMARY

The present disclosure addresses at least the above needs by providing a microelectrode array comprising a plurality of elongated microwires, and methods for fabricating the same. The microelectrode array may be part of a neural-interface probe, and may be implantable on or into a human brain.

According to an aspect, an implantable device may be provided. The method may comprise a substrate comprising a plurality of feedthroughs, wherein the plurality of feedthroughs may comprise a first conductive material; and an array of microwires extending from the substrate, wherein the array of microwires may be connected or bonded to the plurality of feedthroughs using a biocompatible solder or braze material or intermediate filler material, and wherein the array of microwires may comprise a second conductive material that may be different from the first conductive material.

In some embodiments, the substrate may comprise ceramic. In some embodiments, a thickness of the substrate may be equal to or less than about 1 millimeter (mm). In some embodiments, a diameter of each of the plurality of feedthroughs may be from about 25 microns to about 250 microns. In some embodiments, the plurality of feedthroughs may be completely filled with the first conductive material. In some embodiments, sidewalls of the plurality of feedthroughs may be coated with the first conductive material.

In some embodiments, each microwire in the array of microwires may have a conical tip. In some embodiments, a radius of the conical tip may be less than about 5 micrometers. In some embodiments, each microwire in the array of microwires may have a diameter of about 10 micrometers to about 50 micrometers. In some embodiments, each microwire in the array of microwires may have a diameter that decreases monotonically from a proximal end to a distal end of the microwire. In some embodiments, the proximal end of the microwire may be located closer to the substrate than the distal end. In some embodiments, the proximal end of the microwire may have a flange that may be at least about 50% of a diameter of the feedthrough on which the microwire may be located. In some embodiments, wherein a diameter of the distal end of the microwire may be less than about 50% of a diameter of the feedthrough on which the microwire may be located.

In some embodiments, a thickness of the biocompatible solder or braze material may be less than about 200 micrometers. In some embodiments, the biocompatible solder or braze material may be configured to connect the array of microwires to the plurality of feedthroughs without causing electrical shorting between adjacent feedthroughs.

In some embodiments, the device may further comprise a ceramic film coated over or onto the array of microwires. In some embodiments, a thickness of the ceramic film may be about 500 nanometers to about 2 micrometers. In some embodiments, the ceramic film may be further coated on one side of the substrate. In some embodiments, each microwire in the array of microwires may have a de-insulated tip. In some embodiments, the de-insulated tip may have an impedance of about 50 kilo-ohms to about 5000 kilo-ohms when tested at a frequency of about 1 KHz in a biological saline solution. In some embodiments, the array of microwires may be configured to be inserted into brain tissue.

In another aspect, a method of producing an implantable device may be provided. The method may comprise (a) providing a substrate comprising a plurality of feedthroughs, wherein the plurality of feedthroughs may be hermetically formed and may have a leak rate equal to or less than about 10−8 atm*cc/s; (b) bonding a conductive block to the substrate using a biocompatible solder or braze material; and (c) subtracting one or more portions of the conductive block in one or more directions to form an array of microwires extending from the plurality of feedthroughs on the substrate, without affecting the hermiticity and leak rate of the feedthroughs.

In some embodiments, the method of producing an implantable device may further comprise coating the array of microwires and the substrate with a ceramic film. In some embodiments, a thickness of the ceramic film may be about 500 nanometers to about 2 micrometers.

In some embodiments, the method of producing an implantable device may further comprise using a subtractive technique to expose and de-insulate distal tips of the microwires. In some embodiments, the subtractive technique may comprise laser or ion milling. In some embodiments, the de-insulated distal tips of the microwires may have an impedance of about 50 kilo-ohms to about 5000 kilo-ohms when tested at a frequency of about 1 KHz in a biological saline solution.

In some embodiments, the plurality of feedthroughs may comprise a first conductive material, and wherein the array of microwires may comprise a second conductive material that may be different from the first conductive material. In some embodiments, the plurality of feedthroughs may be completely filled with the first conductive material. In some embodiments, sidewalls of the plurality of feedthroughs may be coated with the first conductive material. In some embodiments, the substrate may comprise ceramic. In some embodiments, a thickness of the substrate may be equal to or less than about 1 millimeter (mm). In some embodiments, a diameter of each of the plurality of feedthroughs may be from about 25 microns to about 250 microns.

In some embodiments, the method of producing an implantable device may further comprise modifying distal ends of the microwires such that each distal end may comprise a conical tip or a pyramidal tip. In some embodiments, each distal end may comprise conical tip, and a radius of the conical tip may be less than about 5 micrometers. In some embodiments, each microwire in the array of microwires may have a diameter of about 10 micrometers to about 50 micrometers. In some embodiments, the one or more portions of the conductive block may be subtracted such that each microwire in the array of microwires may have a diameter that decreases monotonically from a proximal end to a distal end of the microwire.

In some embodiments, the proximal end of the microwire may be located closer to the substrate than the distal end. In some embodiments, the proximal end of the microwire may have a flange that may be at least about 50% of a diameter of the feedthrough on which the microwire may be located. In some embodiments, a diameter of the distal end of the microwire may be less than about 50% of a diameter of the feedthrough on which the microwire may be located. In some embodiments, a thickness of the biocompatible solder or braze material may be less than about 200 micrometers. In some embodiments, the array of microwires may be insertable into brain tissue.

In another aspect, a method for forming a microwire array configured for use in a neural interface probe may be provided. The method may comprise (a) providing a substrate, wherein the substrate may comprise a hermetic feedthrough plate comprising a plurality of conductive feedthroughs; (b) bonding the substrate to a block of material using one or more of the following processes: (i) diffusion bonding, (ii) intermediate layer reflow, (iii) ultrasonic bonding/welding, (iv) friction welding, (v) electric welding, or (vi) vacuum cementing; and (c) forming a plurality of microwires on the substrate by applying one or more subtractive processes to the block, wherein the one or more subtractive processes may comprise wire electric discharge machining (EDM), die-sinking EDM, electrochemical machining (ECM), micro-electrochemical machining (microECM), or deep drilling.

In some embodiments, the one or more subtractive processes to the block may be configured to remove one or more portions of the block without substantially affecting the hermetic feedthrough plate or the plurality of conductive feedthroughs.

In another aspect, a method for forming a microwire array configured for use in a neural interface probe may be provided. The method may comprise (a) providing a substrate, wherein the substrate may comprise a hermetic feedthrough plate comprising a plurality of conductive feedthroughs; and (b) forming a plurality of microwires on the substrate by using one or more additive processes, wherein the one or more additive processes may comprise laser sintering, local electrochemical deposition, photolithography-based layer-by-layer (LBL) manufacturing, or 3-D printing.

In some embodiments, the plurality of microwires may comprise heterogeneous microwires comprising two or more different types of materials at different portions of the microwires. In some embodiments, the one or more additive processes may be used to deposit or stack the two or more different types of materials to form the heterogeneous microwires.

In some embodiments, the two or more different types of materials may be configured to impart different physical, chemical and/or electrical properties to the different portions of the microwires.

In some embodiments, the one or more additive processes or the subtractive processes may comprise forming one or more electrical circuits between a plurality of electrodes. In some embodiments, the one or more electrical circuits may be configured to provide control over one or more individual channels for assisting the additive processes or the subtractive processes. In some embodiments, the one or more individual channels may be connected to one or more feedthroughs of the hermetic feedthrough plate. In some embodiments, the one or more electrical circuits may comprise one or more active elements comprising of a voltage controller or a current controller.

In some embodiments, at least one of the subtractive or additive processes may comprise forming a temporary conductive layer on a backside of the hermetic feedthrough plate. In some embodiments, the temporary conductive layer may comprise another block that may be bonded to the backside of the hermetic feedthrough plate. In some embodiments, the temporary conductive layer may be deposited on the backside of the hermetic feedthrough plate using chemical vapor deposition (CVD), physical vapor deposition (PVD), or an electrochemical process.

In another aspect, a method for forming a microwire array may be provided. The method may comprise (a) bonding a substrate to a block, wherein the substrate may comprise a hermetic feedthrough substrate, and wherein the bonding may comprise diffusion bonding; (b) machining a portion of the block along a first direction to form a plurality of extended protrusions extending from the substrate, wherein the plurality of extended protrusions may be separated by a plurality of spaces located therebetween; (c) applying a support material into the plurality of spaces; (d) machining the plurality of extended protrusions along a second direction to form a plurality of microwires extending from the substrate, wherein the second direction may be different from the first direction; and (e) removing the support material from the plurality of spaces to expose the plurality of microwires extending from the substrate.

In some embodiments, the hermetic feedthrough substrate exhibits a high level of hermeticity such that the substrate may be substantially impermeable to fluids and ions. In some embodiments, the block may comprise a conductive material. In some embodiments, the conductive material may be platinum-iridium. In some embodiments, the block may comprise a plurality of insulated portions formed on a surface of the block. In some embodiments, the surface of the block on which the insulated portions may be formed may be opposite to the portion that may be machined.

In some embodiments, the support material may comprise gold, indium, or tin. In some embodiments, the support material may comprise a solder alloy. In some embodiments, the support material may comprise a polymer resin. In some embodiments, the support material may be biocompatible. In some embodiments, the support material may be applied into the plurality of spaces using a reflow process. In some embodiments, the support material may be applied into the plurality of spaces using a needle dispense process. In some embodiments, the support material may be configured to constrain movement of the plurality of extended protrusions and provide structural support as the extended protrusions may be being machined in (c). In some embodiments, the support material may be removed from the plurality of spaces without affecting a position, orientation or structural integrity of the plurality of microwires. In some embodiments, the support material may be removed using an etchant that preferentially etches the support material over the conductive material.

In some embodiments, the method for forming a microwire array may further comprise bonding the substrate to a plurality of bond pads on a chip. In some embodiments, the block may be machined using an electric discharge machining (EDM) process. In some embodiments, the method for forming a microwire array may further comprise forming a plurality of sharpened tips on the plurality of microwires. In some embodiments, the first direction and the second direction may be orthogonal to each other. In some embodiments, the first direction and the second direction may be non-orthogonal to each other.

In another aspect, a method for forming a microwire array may be provided. The method may comprise (a) machining a portion of a block along one or more directions to form a first set of extended protrusions extending from a substrate, wherein the substrate may comprise a hermetic feedthrough substrate, and wherein the first set of extended protrusions may be spaced apart by a first gap; (b) applying a support material into the first gap; and (c) machining the first set of extended protrusions along one or more other directions to form a second set of extended protrusions extending from the substrate, wherein the second set of extended protrusions may be spaced apart by a second gap, wherein a dimension of the first set of extended protrusions may be greater than a dimension of the second set of extended protrusions, and wherein a width of the first gap may be less than a width of the second gap.

In another aspect, a method for forming a microwire array configured for use in a neural interface probe is provided. The method comprises: (a) providing a substrate, wherein the substrate comprises a hermetic feedthrough plate comprising a plurality of conductive feedthroughs; (b) bonding the substrate to a block of material using one or more of the following processes: (i) diffusion bonding, (ii) intermediate layer reflow, (iii) ultrasonic bonding/welding, (iv) friction welding, (v) electric welding, or (vi) vacuum cementing; and (c) forming a plurality of microwires on the substrate by applying one or more subtractive processes to the block, wherein the one or more subtractive processes comprises wire electric discharge machining (EDM), die-sinking EDM, electrochemical machining (ECM), micro-electrochemical machining (microECM), or deep drilling.

In some embodiments, the one or more subtractive processes to the block may be configured to remove one or more portions of the block without substantially affecting the hermetic feedthrough plate or the plurality of conductive feedthroughs.

In another aspect, a method for forming a microwire array configured for use in a neural interface probe is provided. The method comprises: (a) providing a substrate, wherein the substrate comprises a hermetic feedthrough plate comprising a plurality of conductive feedthroughs; and (b) forming a plurality of microwires on the substrate by using one or more additive processes, wherein the one or more additive processes comprises laser sintering, local electrochemical deposition, photolithography-based layer-by-layer (LBL) manufacturing, or 3-D printing.

In some embodiments, the plurality of microwires may comprise heterogeneous microwires comprising two or more different types of materials at different portions of the microwires. The one or more additive processes may be used to deposit or stack the two or more different types of materials to form the heterogeneous microwires. The two or more different types of materials may be configured to impart different physical, chemical and/or electrical properties to the different portions of the microwires.

In some embodiments, the one or more additive processes or the subtractive processes may comprise forming one or more electrical circuits between a plurality of electrodes. The one or more electrical circuits can be configured to provide control over one or more individual channels for assisting the additive processes or the subtractive processes. The one or more individual channels may be connected to one or more feedthroughs of the hermetic feedthrough plate. The one or more electrical circuits may comprise one or more active elements comprising of a voltage controller or a current controller. In some embodiments, at least one of the subtractive or additive processes comprises forming a temporary conductive layer on a backside of the hermetic feedthrough plate. The temporary conductive layer may comprise another block that is bonded to the backside of the hermetic feedthrough plate. The temporary conductive layer may be deposited on the backside of the hermetic feedthrough plate using chemical vapor deposition (CVD), physical vapor deposition (PVD), or an electrochemical process.

In another aspect, a method for forming a microwire array is provided. The method comprises: (a) bonding a substrate to a block, wherein the substrate comprises a hermetic feedthrough substrate, and wherein the bonding comprises diffusion bonding; (b) machining a portion of the block along a first direction to form a plurality of extended protrusions extending from the substrate, wherein the plurality of extended protrusions are separated by a plurality of spaces located therebetween; (c) applying a support material into the plurality of spaces; (d) machining the plurality of extended protrusions along a second direction to form a plurality of microwires extending from the substrate, wherein the second direction is different from the first direction; and (e) removing the support material from the plurality of spaces to expose the plurality of microwires extending from the substrate.

In some embodiments, the hermetic feedthrough substrate may exhibit a high level of hermeticity such that the substrate is substantially impermeable to fluids and ions. The block may comprise a conductive material. In some cases, the conductive material may be platinum-iridium.

In some embodiments, the block may comprise a plurality of insulated portions formed on a surface of the block. The surface of the block on which the insulated portions is formed may be opposite to the portion that is machined. In some cases, the support material may comprise gold, indium, or tin. In some cases, the support material may comprise a solder alloy. In some cases, the support material may comprise a polymer resin. In some cases, the support material may be biocompatible.

In some embodiments, the support material can be applied into the plurality of spaces using a reflow process. In some cases, the support material can be applied into the plurality of spaces using a needle dispense process. The support material can be configured to constrain movement of the plurality of extended protrusions and provide structural support as the extended protrusions are being machined in (c). The support material can be removed from the plurality of spaces without affecting a position, orientation or structural integrity of the plurality of microwires. The support material can be removed using an etchant that preferentially etches the support material over the conductive material.

In some embodiments, the method may further comprise: bonding the substrate to a plurality of bond pads on a chip. In some cases, the block can be machined using an electric discharge machining (EDM) process. In some embodiments, the method may further comprise: forming a plurality of sharpened tips on the plurality of microwires.

In some embodiments of the method, the first direction and the second direction may be orthogonal to each other. Alternatively, the first direction and the second direction may be non-orthogonal to each other.

In another aspect, a method for forming a microwire array is provided. The method comprises: (a) machining a portion of a block along one or more directions to form a first set of extended protrusions extending from a substrate, wherein the substrate comprises a hermetic feedthrough substrate, and wherein the first set of extended protrusions are spaced apart by a first gap; (b) applying a support material into the first gap; and (c) machining the first set of extended protrusions along one or more other directions to form a second set of extended protrusions extending from the substrate, wherein the second set of extended protrusions are spaced apart by a second gap, wherein a dimension of the first set of extended protrusions is greater than a dimension of the second set of extended protrusions, and wherein a width of the first gap is less than a width of the second gap.

In another aspect, a method for forming a microwire array is provided. The method comprises: (a) machining a portion of a block along a first direction to form a plurality of extended protrusions, wherein the plurality of extended protrusions are separated by a plurality of spaces located therebetween; (b) applying a support material into the plurality of spaces; (c) machining the plurality of extended protrusions along a second direction to form a plurality of microwires on a substrate, wherein the second direction is different from the first direction; and (d) removing the support material from the plurality of spaces to expose the plurality of microwires on the substrate.

In another aspect, a method for forming a microwire array configured for use in a neural interface probe is provided. The method comprises: (a) providing a substrate, wherein the substrate comprises a hermetic feedthrough plate comprising a plurality of conductive feedthroughs; (b) bonding the substrate to a block of material using one or more of the following processes: (i) diffusion bonding, (ii) intermediate layer reflow, (iii) ultrasonic bonding/welding, (iv) friction welding, (v) electric welding, or (vi) vacuum cementing; and (c) forming a plurality of microwires on the substrate by applying one or more subtractive processes to the block, wherein the one or more subtractive processes comprises wire electric discharge machining (EDM), die-sinking EDM, electrochemical machining (ECM), micro-electrochemical machining (microECM), or deep drilling.

In some embodiments, the one or more subtractive processes to the block may be configured to remove one or more portions of the block without substantially affecting the hermetic feedthrough plate or the plurality of conductive feedthroughs.

In another aspect, a method for forming a microwire array configured for use in a neural interface probe is provided. The method comprises: (a) providing a substrate, wherein the substrate comprises a hermetic feedthrough plate comprising a plurality of conductive feedthroughs; and (b) forming a plurality of microwires on the substrate by using one or more additive processes, wherein the one or more additive processes comprises laser sintering, local electrochemical deposition, photolithography-based layer-by-layer (LBL) manufacturing, or 3-D printing.

In some embodiments, the plurality of microwires may comprise heterogeneous microwires comprising two or more different types of materials at different portions of the microwires. The one or more additive processes may be used to deposit or stack the two or more different types of materials to form the heterogeneous microwires. The two or more different types of materials may be configured to impart different physical, chemical and/or electrical properties to the different portions of the microwires.

In some embodiments, the one or more additive processes or the subtractive processes may comprise forming one or more electrical circuits between a plurality of electrodes. The one or more electrical circuits can be configured to provide control over one or more individual channels for assisting the additive processes or the subtractive processes. The one or more individual channels may be connected to one or more feedthroughs of the hermetic feedthrough plate. The one or more electrical circuits may comprise one or more active elements comprising of a voltage controller or a current controller. In some embodiments, at least one of the subtractive or additive processes comprises forming a temporary conductive layer on a backside of the hermetic feedthrough plate. The temporary conductive layer may comprise another block that is bonded to the backside of the hermetic feedthrough plate. The temporary conductive layer may be deposited on the backside of the hermetic feedthrough plate using chemical vapor deposition (CVD), physical vapor deposition (PVD), or an electrochemical process.

In another aspect, a method for forming a microwire array is provided. The method comprises: (a) bonding a substrate to a block, wherein the substrate comprises a hermetic feedthrough substrate, and wherein the bonding comprises diffusion bonding; (b) machining a portion of the block along a first direction to form a plurality of extended protrusions extending from the substrate, wherein the plurality of extended protrusions are separated by a plurality of spaces located therebetween; (c) applying a support material into the plurality of spaces; (d) machining the plurality of extended protrusions along a second direction to form a plurality of microwires extending from the substrate, wherein the second direction is different from the first direction; and (e) removing the support material from the plurality of spaces to expose the plurality of microwires extending from the substrate.

In some embodiments, the hermetic feedthrough substrate may exhibit a high level of hermeticity such that the substrate is substantially impermeable to fluids and ions. The block may comprise a conductive material. In some cases, the conductive material may be platinum-iridium.

In some embodiments, the block may comprise a plurality of insulated portions formed on a surface of the block. The surface of the block on which the insulated portions is formed may be opposite to the portion that is machined. In some cases, the support material may comprise gold, indium, or tin. In some cases, the support material may comprise a solder alloy. In some cases, the support material may comprise a polymer resin. In some cases, the support material may be biocompatible.

In some embodiments, the support material can be applied into the plurality of spaces using a reflow process. In some cases, the support material can be applied into the plurality of spaces using a needle dispense process. The support material can be configured to constrain movement of the plurality of extended protrusions and provide structural support as the extended protrusions are being machined in (c). The support material can be removed from the plurality of spaces without affecting a position, orientation or structural integrity of the plurality of microwires. The support material can be removed using an etchant that preferentially etches the support material over the conductive material.

In some embodiments, the method may further comprise: bonding the substrate to a plurality of bond pads on a chip. In some cases, the block can be machined using an electric discharge machining (EDM) process. In some embodiments, the method may further comprise: forming a plurality of sharpened tips on the plurality of microwires.

In some embodiments of the method, the first direction and the second direction may be orthogonal to each other. Alternatively, the first direction and the second direction may be non-orthogonal to each other.

In another aspect, a method for forming a microwire array is provided. The method comprises: (a) machining a portion of a block along one or more directions to form a first set of extended protrusions extending from a substrate, wherein the substrate comprises a hermetic feedthrough substrate, and wherein the first set of extended protrusions are spaced apart by a first gap; (b) applying a support material into the first gap; and (c) machining the first set of extended protrusions along one or more other directions to form a second set of extended protrusions extending from the substrate, wherein the second set of extended protrusions are spaced apart by a second gap, wherein a dimension of the first set of extended protrusions is greater than a dimension of the second set of extended protrusions, and wherein a width of the first gap is less than a width of the second gap.

In another aspect, a method for forming a microwire array is provided. The method comprises: (a) machining a portion of a block along a first direction to form a plurality of extended protrusions, wherein the plurality of extended protrusions are separated by a plurality of spaces located therebetween; (b) applying a support material into the plurality of spaces; (c) machining the plurality of extended protrusions along a second direction to form a plurality of microwires on a substrate, wherein the second direction is different from the first direction; and (d) removing the support material from the plurality of spaces to expose the plurality of microwires on the substrate.

According to a further aspect, a method for monitoring and/or stimulating neural activity, may comprise inserting a neural-interface probe comprising any of the aforementioned microwire array into a subject's brain, such that the tips of the plurality of microwires interface and are in contact with an area of neural matter; and monitoring and/or stimulating neural activity in the area via a plurality of electrical signals transmitted between the chip and the neural matter through the plurality of microwires.

Additional aspects and advantages of the present disclosure will become readily apparent to those skilled in this art from the following detailed description, wherein only exemplary embodiments of the present disclosure are shown and described, simply by way of illustration of the different modes contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a patterned substrate in accordance with some embodiments.

FIG. 2 shows a patterned matching block in proximity and aligned with the patterned substrate of FIG. 1, in accordance with some embodiments.

FIG. 3 shows the bonding of the patterned matching block with the patterned substrate, in accordance with some embodiments.

FIG. 4 shows a feedthrough device that is formed after backgrinding of the bonded assembly of FIG. 3, in accordance with some embodiments.

FIG. 5 shows the feedthrough device in proximity with a conductive block, in accordance with some embodiments.

FIG. 6 shows the bonding of the feedthrough device with the conductive block, in accordance with some embodiments.

FIG. 7 shows sharpened tips being formed on a surface of the conductive block, in accordance with some embodiments.

FIG. 8 shows a protective coat provided on the surface of the conductive block covering the sharpened tips, in accordance with some embodiments.

FIG. 9 shows a portion of the conductive block being removed to form elongated protrusions, in accordance with some embodiments.

FIG. 10 shows the elongated protrusions of the conductive block in proximity and aligned with a template plate, in accordance with some embodiments.

FIG. 11 shows the elongated protrusions inserted through the holes of the template plate, in accordance with some embodiments.

FIG. 12 shows additional material of the conductive block being removed to elongate the protrusions, in accordance with some embodiments.

FIG. 13 shows microwires being formed and isolated from one another, in accordance with some embodiments.

FIG. 14 shows the backside of the feedthrough device bonded to a chip to form an active microelectrode array, in accordance with some embodiments.

FIG. 15 shows the sharpened tips of the microwires being inserted into the brain of a subject using the template plate for guiding the tips of the microwires, in accordance with some embodiments.

FIG. 16 shows a perspective view of the microprobe array of FIG. 14 without the template plate, in accordance with some embodiments.

FIG. 17 shows a perspective view of the microprobe array of FIG. 14 with the template plate, in accordance with some embodiments.

FIGS. 18A-18E illustrate a process for fabricating a microwire array, in accordance with some embodiments.

FIGS. 19A-19G illustrate examples of other processes for fabricating a microwire array, in accordance with some other embodiments.

FIG. 20 illustrates a cross section of a microwire array, in accordance with some embodiments.

FIGS. 21A-21F illustrate a process for improving support during fabrication of a microwire array, in accordance with some embodiments.

FIG. 22 illustrates a patterned substrate in accordance with some embodiments.

FIG. 23 shows a patterned matching block in proximity and aligned with the patterned substrate of FIG. 22, in accordance with some embodiments.

FIG. 24 shows the bonding of the patterned matching block with the patterned substrate, in accordance with some embodiments.

FIG. 25 shows a feedthrough device that is formed after backgrinding of the bonded assembly of FIG. 24, in accordance with some embodiments.

FIG. 26 shows the feedthrough device in proximity with a conductive block, in accordance with some embodiments.

FIG. 27 shows the bonding of the feedthrough device with the conductive block, in accordance with some embodiments.

FIG. 28 shows additional material of the conductive block being removed to form elongated protrusions, in accordance with some embodiments.

FIGS. 29A and 29B show a process for improving support during fabrication of a microwire array, in accordance with some embodiments.

FIGS. 30A and 30B show microwires being formed and isolated from one another, in accordance with some embodiments.

FIGS. 31A and 31B illustrate examples of other processes for fabricating a microwire array (sectional view), in accordance with some other embodiments.

FIGS. 32A-32C illustrate examples of other processes for fabricating a microwire array (planar view), in accordance with some other embodiments.

FIG. 33 illustrates a perspective view of the microprobe array, in accordance with some embodiments.

FIGS. 34A and 34B illustrate a process for fabricating a microwire array, in accordance with some embodiments.

FIGS. 35A-35E show another example of a process for fabricating a microwire array, in accordance with some other embodiments.

FIGS. 36A-36D illustrate another example of a process for fabricating a microwire array, in accordance with some other embodiments.

FIG. 37 shows an example of a circuit configured to form electrodes, in accordance with some embodiments.

FIG. 38A shows an example of connecting feedthroughs using a conductive block, in accordance with some embodiments.

FIG. 38B shows an example of connecting feedthroughs using a thin film, in accordance with some embodiments.

FIGS. 39A-39C illustrate a circuit connection to the feedthrough plate, in accordance with some embodiments.

FIGS. 40A-40D show examples of connecting a fixture to a feedthrough plate, in accordance with some embodiments.

FIGS. 41A-41D illustrate an example of a circuit configured to form electrodes, in accordance with some embodiments.

FIG. 42A-42C illustrates examples of neural interface probe devices, in accordance with some embodiments.

FIG. 43A-43C illustrate other examples of neural interface probe devices, in accordance with some embodiments.

### DETAILED DESCRIPTION

The present disclosure is directed to a microelectrode array comprising a plurality of elongated flexible microwires, and methods for fabricating the same. The microelectrode array may be part of a neural-interface probe, and may be implantable on or into a human brain. The microelectrode array may comprise a wire bundle having a plurality of wires configured to interface with neural matter. The terms “wire(s),” “microwire(s),” “microprobe(s),” “probe(s),” “microelectrode(s),” and “electrode(s)” may be used interchangeably herein. The wires in the wire bundle are configured to be electrically interconnected with a chip. The chip may be configured to stimulate and/or monitor brain activity. In some instances, the chip may be an integrated circuit imaging chip capable of recording neural signals from areas and/or curved surfaces within a brain. In some cases, the wires of the wire bundle may be individually addressable, such that one or more wires can be configured to provide multi-site, spatially controlled stimulation of neural matter. For example, the chip may comprise a plurality of pixels controlling a plurality of electrodes. One or more wires of the wire bundle may be connected to each pixel. The stimulation frequency and amplitude of each electrode can be individually fine-tuned to control the pixels.

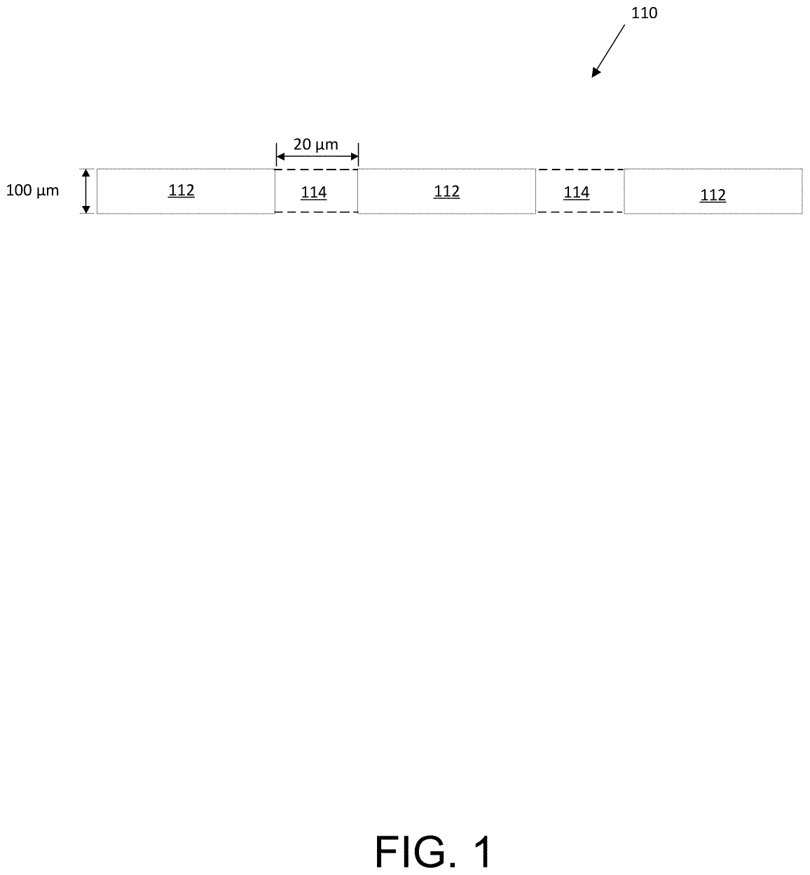

FIG. 1 illustrates a patterned substrate 110 in accordance with some embodiments. The term “substrate,” as used herein, generally refers to any substance to which other materials can be bonded, or upon which a layered structure can be deposited. The substrate 110 may comprise a solid material such as a semiconductor or an insulator. The substrate material may be single crystalline, poly crystalline, or amorphous. Substrate materials may comprise, for example, sapphire, silicon, silicon dioxide, silicon carbide, aluminum oxide, aluminum nitride, germanium, gallium arsenide, gallium nitride, indium phosphide, diamond, or synthetic diamond. In some embodiments, substrate materials may comprise silicon, gallium, carbon, germanium, arsenic, thallium, cadmium, tellurium, selenium, or alloy or allotrope thereof, or an oxide or nitride thereof. In some embodiments, the substrate may include one or more chemical dopants, such as nitrogen, phosphorous, boron or indium. Referring to FIG. 1, the patterned substrate 100 may comprise a plurality of patterned portions 112 and holes 114. The holes may be through-holes. The holes may be formed using a variety of processes, for example laser drilling, etching (e.g. deep reactive ion etching), dry etch or wet etch processes, additive processes such as 3D printing, molding or sintering, etc. In some preferred embodiments, the patterned substrate 110 may be made of sapphire, and the holes may be formed by laser drilling through an unpatterned sapphire substrate. The patterned substrate 110 may be formed having a thickness ranging from 5 um to 1 mm. The holes 114 may be formed having a size (e.g. width or diameter) ranging from 5 um to 100 um. In some embodiments, the thickness of the patterned substrate 110 may be about 100 um, and the holes may have a size of about 20 um.

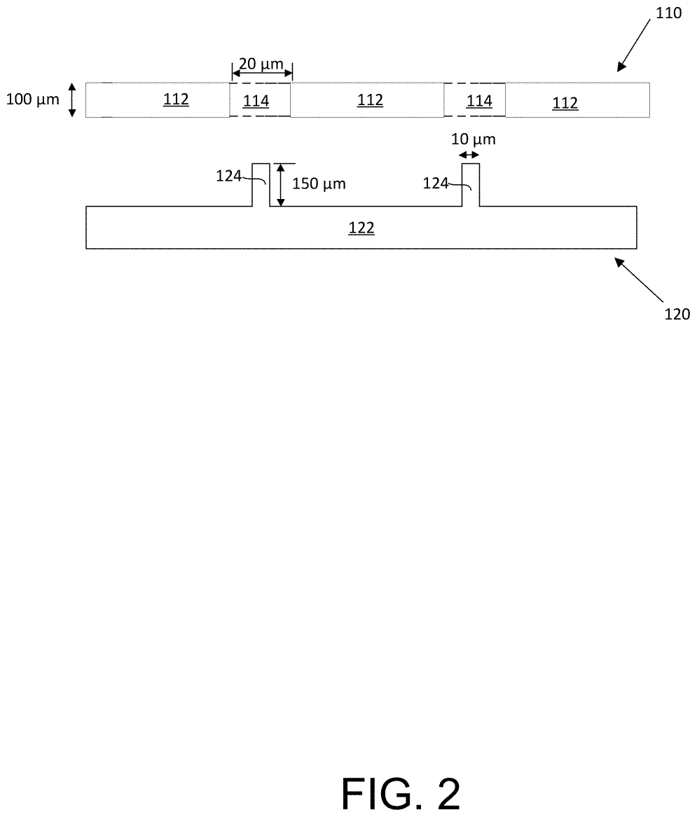

FIG. 2 shows a patterned matching block 120 in proximity and aligned with the patterned substrate 110 of FIG. 1, in accordance with some embodiments. In some embodiments, the patterned matching block 120 may be made of a material comprising a transition metal. In some embodiments, the material may comprise niobium, chromium, scandium, titanium, vanadium, manganese, iron, cobalt, nickel, copper, zinc, yttrium, zirconium, platinum, gold, mercury, iridium, molybdenum, silver, tantalum, tungsten, aluminum, silicon, phosphorous, tin, an oxide of any of the preceding or any combination thereof. In other embodiments, the material may be a conductive ceramic such as TiN, conductive SiN, Indium tin oxide, etc. The patterned matching block 120 may be formed having a pattern that matches with the patterned substrate 110. The patterned matching block 120 may comprise a base portion 122, and a plurality of pins (or pillars) 124 on the base portion. The pins 124 may extend or protrude from a surface of the base portion. The pins may have a height ranging from 50 um to 1 mm, and a width (or diameter) ranging from 2 um to 90 um. In some embodiments, the pins may have a height of about 150 um, and a width (or diameter) of about 10 um. In some preferred embodiments, the patterned matching block 120 may be made of niobium, and the pins may be formed by etching a niobium block using a mask. In some instances, the pins may be formed by electric discharge machining.

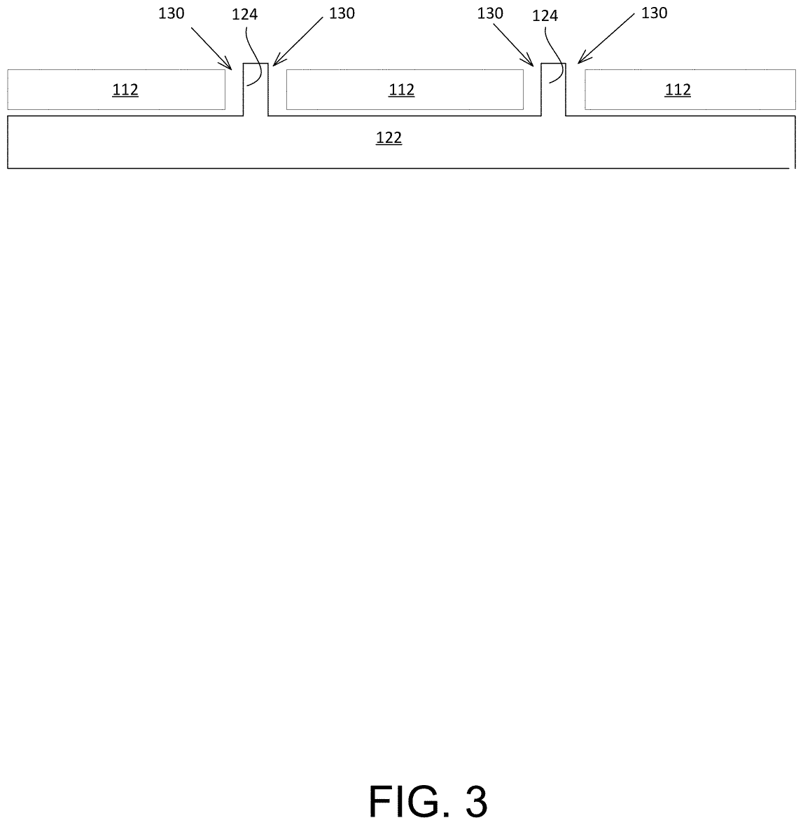

FIG. 3 shows the bonding of the patterned matching block 120 with the patterned substrate 110, in accordance with some embodiments. When the pins of the patterned matching block 120 are aligned with the holes of the patterned substrate 110, the patterned matching block 120 and the patterned substrate 110 are brought into proximity with each other such that the pins 124 are located in the holes 114, as shown in FIG. 3. Next, the patterned matching block 120 and the patterned substrate 110 are bonded to each other, by reflowing a bonding material 130 into the gaps between the patterned matching block 120 and the patterned substrate 110. The bonding material may comprise a conductive material that has a relatively low melting point, and that is capable of filling the gaps completely. In some embodiments, the bonding material may comprise gold. In some cases, the reflow of the bonding material and the bonding process may be performed in a low pressure environment under vacuum.

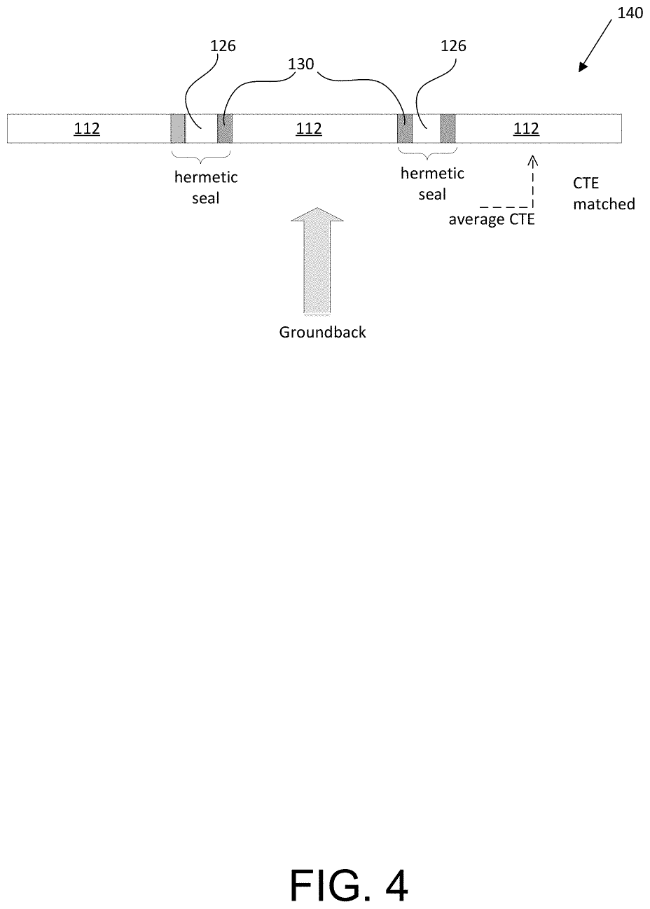

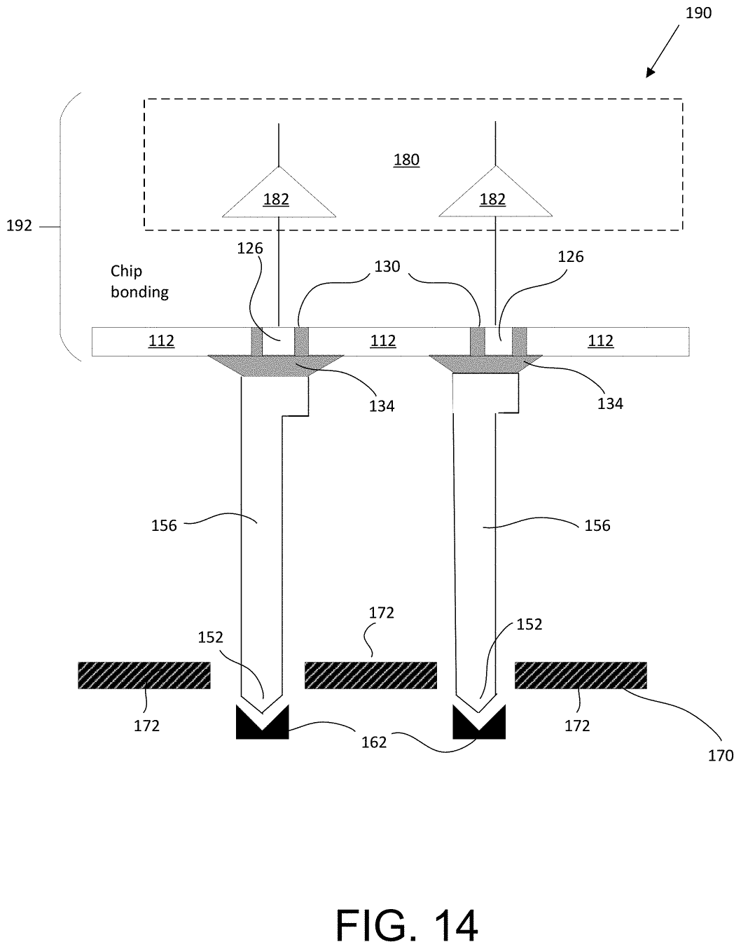

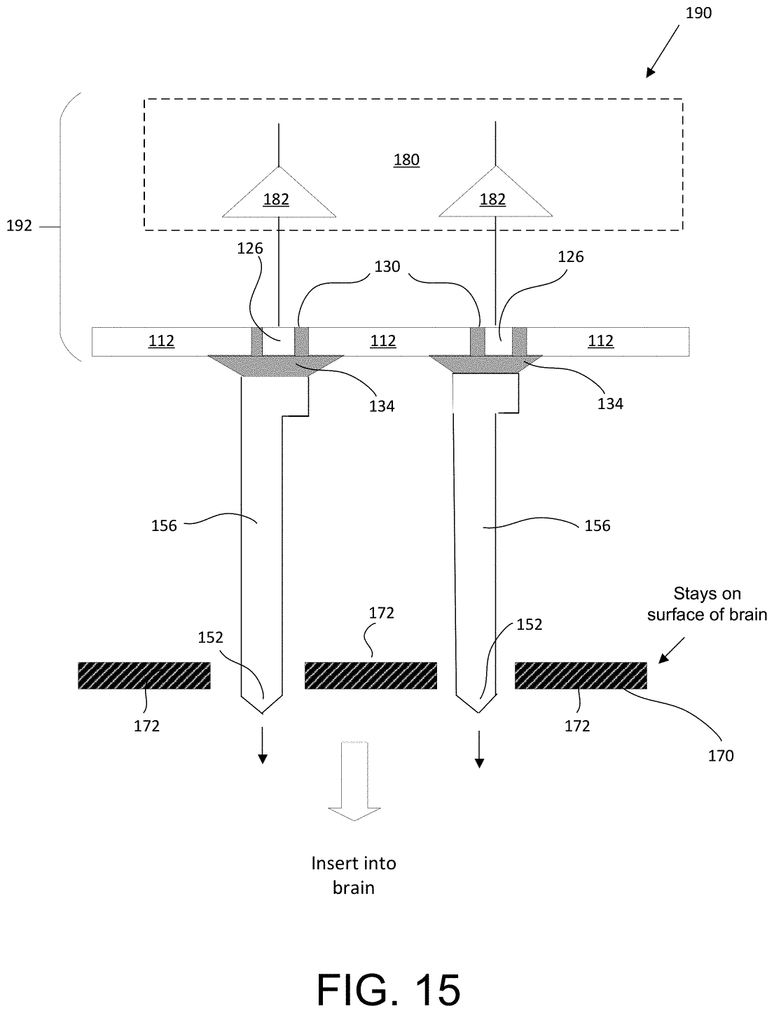

FIG. 4 shows a feedthrough device 140 that is formed after backgrinding of the bonded assembly of FIG. 3, in accordance with some embodiments. The backgrinding may be performed on one side, or both opposite sides of the bonded assembly. The backgrinding may be performed using any bulk etch back or grinding/polishing/machining processes, for example chemical mechanical polishing (CMP). The backgrinding also enables planarization on both surfaces of the feedthrough device 140. In some embodiments, the feedthrough device 140 may have a thickness of about or less than 100 um. Referring to FIG. 4, the feedthrough device 140 may comprise a plurality of conductive pads 126 spaced apart and separated from one another by insulating portions. The conductive pads 126 may include the backgrinded remaining portion of the pins 124. The insulating portions may comprise the patterned portions 112 of the patterned substrate 110. In some embodiments, the patterned portions 112 may comprise sapphire. In some embodiments, the conductive pads 126 may comprise niobium which is bonded to the patterned portions 112 via the bonding material 130 (e.g. gold). The bonding material 130 is configured to form a hermetic seal that prevents any fluid or ions from flowing through the feedthrough device 140. The feedthrough device 140 may be bonded to a chip, as described in more detail with reference to FIGS. 14 and 15. The hermetic sealing can help to prevent fluids from leaking into and entering the chip which can cause electrical failures. The bonding material 130 is also configured to reduce a coefficient of thermal expansion (CTE) mismatch between the bonded conductive pads 126 and the patterned portions 112, which are made of different materials and may be subject to thermomechanical stresses. This may be achieved by selecting dimensions such that the weighted average CTE of the materials 126 and 130 match the CTE of material 112.

It should be appreciated that there may be other methods for forming the feedthrough device 140. In some embodiments, a feedthrough device may be formed by creating through holes or through vias in a substrate, or machining holes in a substrate. Next, the conductive pads may be formed by filling the through vias or holes with a conductive material. The conductive material may be electroplated to form the conductive pads. In some instances, the conductive material (e.g. metal particles) may be reflowed to form the conductive pads.

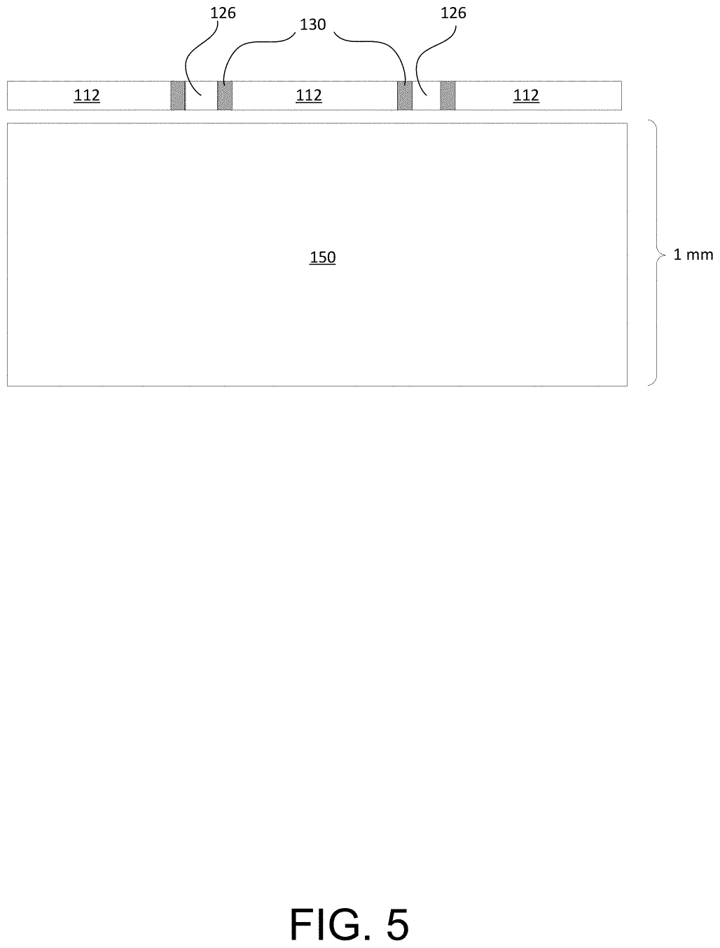

FIG. 5 shows the feedthrough device 140 in proximity with a conductive block 150, in accordance with some embodiments. The conductive block 150 may comprise a metal or metal alloy, for example platinum, iridium, niobium, chromium, scandium, titanium, vanadium, manganese, iron, cobalt, nickel, copper, zinc, yttrium, zirconium, gold, mercury, molybdenum, silver, tantalum, tungsten, aluminum, silicon, phosphorous, tin, an oxide of any of the preceding or any combination thereof. In other embodiments, the material may be a conductive ceramic such as TiN, conductive SiN, Indium tin oxide, etc.

In some embodiments, the conductive block 150 may comprise a same material as the patterned matching block 120. In other embodiments, the conductive block 150 and the patterned matching block 120 may comprise different materials. For example, in some embodiments, the conductive block 150 may comprise a platinum-iridium alloy, and the patterned matching block 120 may comprise niobium. In some embodiments, the conductive block 150 may comprise two or materials. For example, the conductive block 150 may comprise a first portion made of tungsten, and a second portion made of platinum iridium. The first and second portions may be bonded or fused to each other, for example using compression bonding or friction welding. The first portion (e.g. tungsten) may constitute a bulk of the conductive block, and may be used to provide stiffness/rigidity along the length of the microwires to be fabricated. The second portion (e.g. platinum iridium) may be used for the tips of the microwires, and may comprise a material that allows for enhanced neuronal recording. In some embodiments, the conductive block 150 may comprise a titanium-aluminum-vanadium alloy. It should be appreciated that two or more blocks of different materials (or alternating same materials) can be fused together to form the conductive block 150. The conductive block 150 may have a thickness ranging from about 100 um to about 2 mm. In some embodiments, the thickness of the conductive block 150 may be about 1 mm.

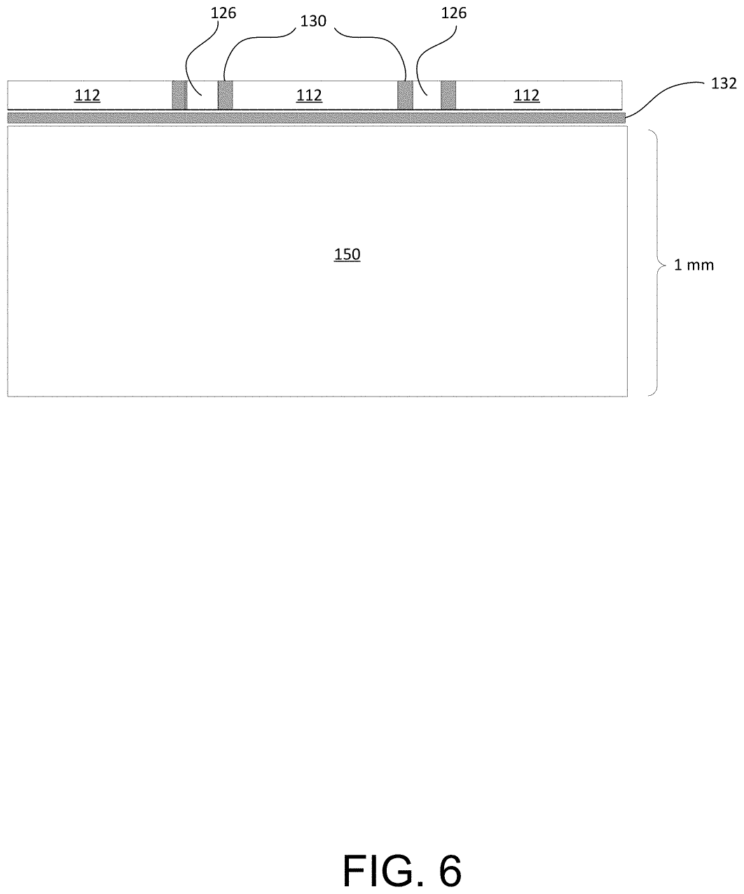

FIG. 6 shows the bonding of the feedthrough device 140 with the conductive block 150, in accordance with some embodiments. The feedthrough device 140 and the conductive block 150 may be brought into proximity with each other, and bonded using a bonding material 132. The bonding material 132 may be similar to the bonding material 130 described elsewhere herein. In some embodiments, the bonding material 132 may comprise gold. The feedthrough device 140 and the conductive block 150 may be bonded to each other by applying pressure and reflowing the bonding material 132 therebetween, for example using thermocompression bonding.

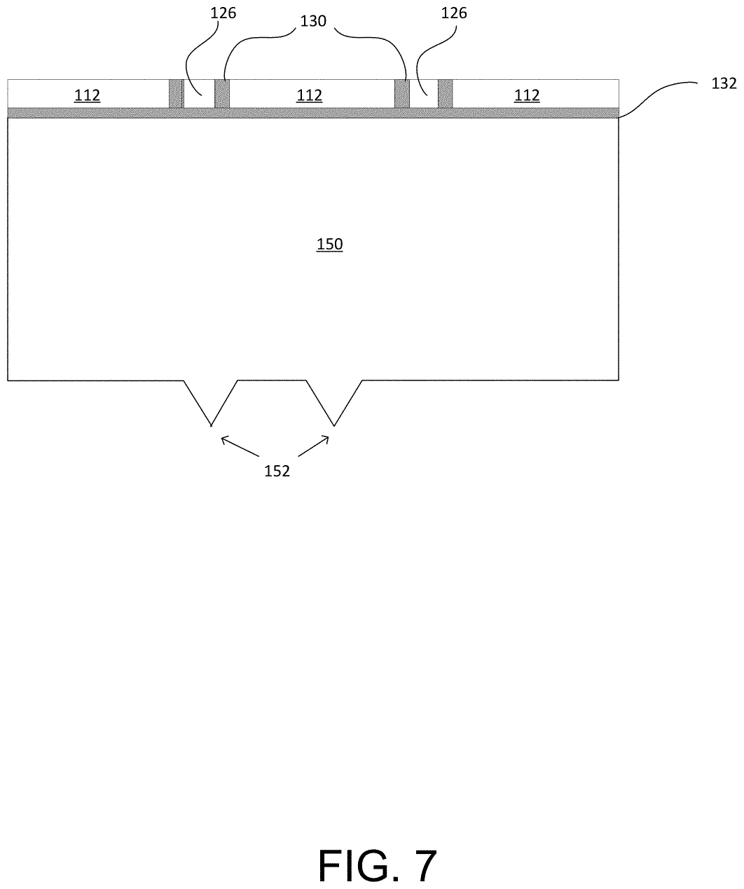

FIG. 7 shows sharpened tips 152 being formed on a surface of the conductive block 150, in accordance with some embodiments. The sharpened tips may be formed by etching the conductive block along a set of predefined planes, by ion beam material removal, by laser milling, by electric discharge machining, or by mechanical milling. The sharpened tips may have a beveled cut surface. In some embodiments, the beveled cut surface may have an angle of about 5 degrees, 10 degrees, 20 degrees, 30 degrees, 40 degrees, 50 degrees, 60 degrees, 70 degrees, 80 degrees, 90 degrees, or any angle within a range between any two of the preceding values. The sharpened tips can help to facilitate insertion into brain tissue, as shown later in FIG. 15.

In some embodiments, a portion of the surface of the conductive block 150 may be subject to an electrochemical modification process, which may comprise tip-shaping of a plurality of protrusions on the conductive block to form sharpened tips.

Electrochemical modification can be carried out in an electrically conductive bath. In the case of subtractive electrochemical modification, the bath may contain reactive elements such as sodium or potassium hydroxide, at a concentration above 0.01M and below 10M. The bath may also contain acids such as sulfuric or phosphoric acid. The subtractive process may be performed under an applied voltage at a metal core, which may typically be positive. The specific formulation used in the bath can vary depending on the selected material, as known to those skilled in the art.

Subtractive modification may result in two types of general geometries: (1) smooth and flat material removal (often called electropolishing), or (2) the sharpening of the wire to an apex (called electrosharpening). The geometries may depend on factors such as mass transport of chemical reagents to the material surface, applied potential at the electrode surface, solution composition, temperature, applied forces to the electrode during the process, and the like.

The additive process may be carried out using a solution that contains the dissolved ions of the material. An electric potential may be applied to the electrode which is undergoing modification, but this may not be necessary. When a potential is applied, it may typically be negative, which causes the dissolved ions to reduce onto the surface of the material as a solid. When a potential is not applied to the electrode undergoing modification, a reducing agent may typically be included in the solution to enable the application of the material coating. Growth of material at the surface of the electrode may take on different forms and morphologies depending on the deposition conditions, including reagent chemistry, chemical mass transport, temperature, electric potential applied to the electrode, and the like. In some embodiments, deposition of the additive solution may be carried out by local deposition using a pipette or micropipette or any other suitable tool that is capable of dispensing volumes from 1 um³ to 10000 um³. The additive solution may also be applied via the use of surface tension, for example by dipping the array into a solution and withdrawing it so that the surface tension of the liquid holds it in place. The additive solution may also be added via condensation, being nucleated at the tip of the wire via an evaporative or Ostwald ripening process. The process may also be carried out by the formation of a mask layer on top of the electrode, after which material can be deposited by a sputtering, evaporation, or electrochemical process.

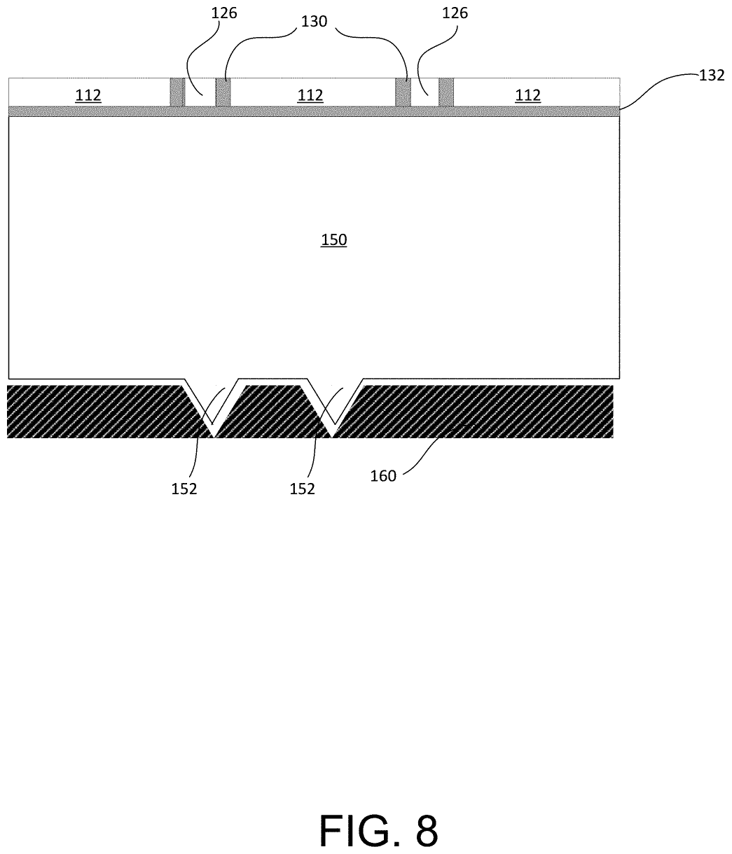

FIG. 8 shows a coating layer 160 provided on the surface of the conductive block 150 covering the sharpened tips 152, in accordance with some embodiments. The coating layer serves to protect the sharpened tips 152 as a portion of the conductive block 150 is removed to form elongated protrusions eventually resulting in a plurality of microwires.

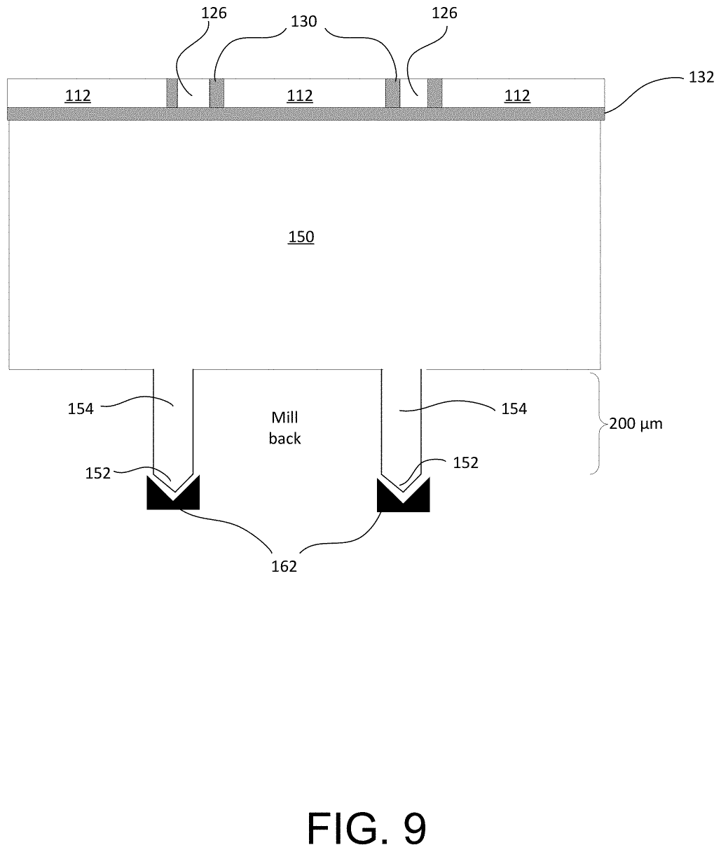

FIG. 9 shows a portion of the conductive block 150 being removed to form elongated protrusions 154, in accordance with some embodiments. A portion of the conductive block 150 can be removed by machining back the conductive block to yield the high aspect ratio elongated protrusions 154. The machining process may include electric discharge machining (EDM), mechanical milling, LIGA, inductively coupled plasma (ICP) etching and the like.

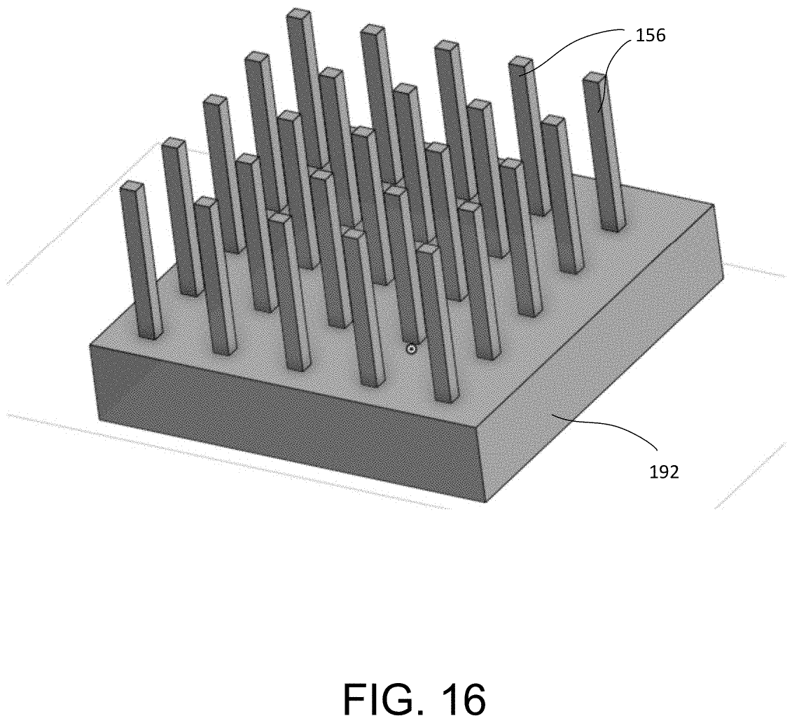

In some embodiments, the conductive block can be milled back using wire-EDM, which is a metal-working process whereby material is removed by an electro-thermal erosion mechanism. Wire-EDM can be used to fabricate metallic microelectrodes with high aspect ratio since there is no cutting force involved in material removal. Wire-EDM generally occurs in a controlled environment and utilizes precision actuation stages to enable positioning with micrometer accuracy. Wire-EDM can be used to cut slits/slots having a relatively small width (e.g. 30 um) and deep (e.g. on the order of several hundred microns) in a metal block. By cutting a set of parallel slits and then rotating the work piece (block) by an angle (e.g. 90 degrees) and repeating the cutting process, a microstructure array can be formed, for example shown in FIG. 16. Although FIG. 16 shows an orthogonal microstructure array, the present disclosure is not limited thereto. For example, three sets of cuts at 60 degrees can be carried out to form a triangular patterned microstructure array.

Referring back to FIG. 9, the coating layer 160 may be removed before or during the cutting process. A portion of the coating layer 160 may remain on the sharpened tips 152 as protective covers 162. The protective covers serve to protect the sharpened tips as the conductive block 150 is being cut back.

Traditional wire-EDM has several process challenges. For example, it can be difficult to mount a small block/workpiece to a worktable. Repositioning and readjusting the workpiece can be tedious and time-consuming if there is more than one surface to be machined. In some cases, it can be difficult to control the positioning and tension of the cutting wire if the aspect ratio is large. For example, the microstructures and the cutting wire may be prone to vibration and unwanted movements during the EDM process, which can affect the geometric accuracy of the microstructures. In addition, microstructures with high aspect ratio may be sensitive to machining heat, and can deform or break due to excessive absorption of Joule heat. Accordingly, the movement and vibration of the microstructures and cutting wire have to be reduced, otherwise geometric accuracy and/or structural deformation of the microstructures may occur during the EDM process.

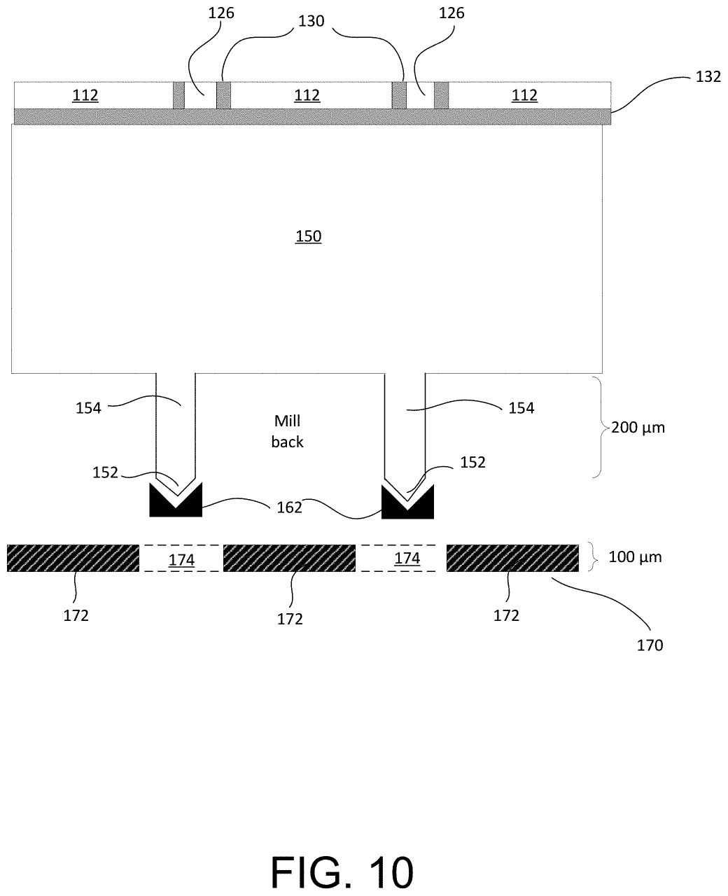

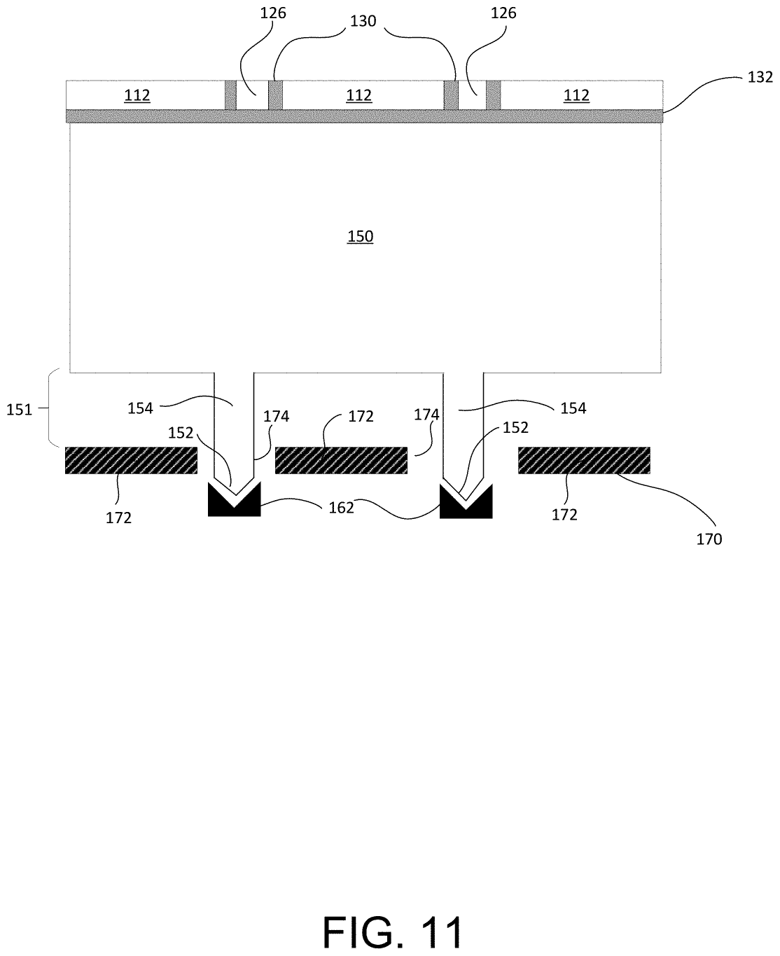

The above challenges associated with wire-EDM can be mitigated through the use of a template plate to constrain vibration and movement of the elongated protrusions (thus allowing for controlled movement of the cutting wire) during the EDM process. FIG. 10 shows the elongated protrusions 154 of the conductive block 150 in proximity and aligned with a template plate 170, and FIG. 11 shows the elongated protrusions 154 inserted through the holes 174 of the template plate, in accordance with some embodiments. The template plate 170 may comprise a plurality of patterned portions 172 and holes 174. The template plate 170 can be used to constrain the ends of the elongated protrusions (i.e. near the sharpened tis 152) as the conductive block 150 is being milled back. The holes 174 in the template plate 170 may be through-holes. The holes may be formed using a variety of processes, for example laser drilling, etching (e.g. deep reactive ion etching), dry etch or wet etch processes, etc. In some embodiments, the template plate 170 may be made of any appropriate material that is biocompatible and suitable for placement onto brain tissue. The biocompatible material may comprise a biocompatible metal that does not easily degrade in a moist environment. In some embodiments, the biocompatible metal may comprise gold, copper, platinum, silver, or any metallic alloy. In some embodiments, the template plate 170 may comprise a semiconductor, a conductive polymer, or a conductive composite material. In some embodiments, the template plate 170 may comprise an insulating material such as silicone compounds (e.g., polydimethylsiloxane (PDMS), poly(methyl methacrylate) (PMMA), etc.), medical-grade epoxies, organic polymer encapsulants, composite materials, and the like. In some embodiments, the material for the template plate may be chosen such that the material is capable of dissolving within a subject's body, and thus disappears after the template plate has served its function of stabilizing the wires during implantation. In some embodiments, the holes in the template plate 170 may be formed by laser drilling through an unpatterned substrate. The template plate 170 may be formed having a thickness ranging from 50 um to 1 mm. The holes 174 may be formed having a size (e.g. width or diameter) ranging from 5 um to 100 um. In some embodiments, the thickness of the template plate 170 may be about 100 um, and the holes 174 may have a size of about 30 um.

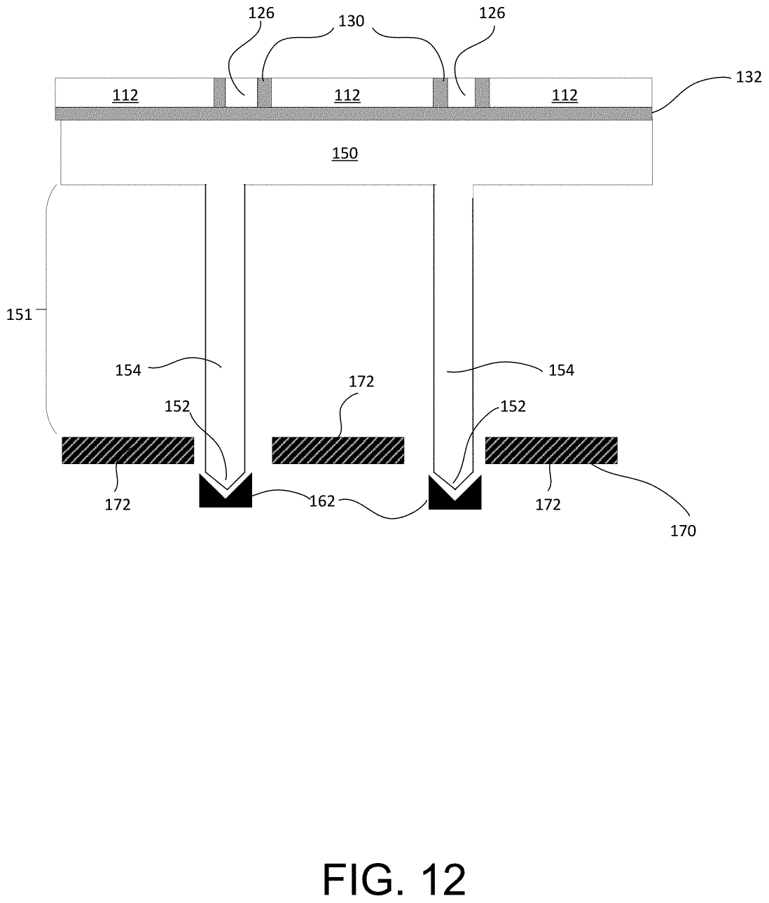

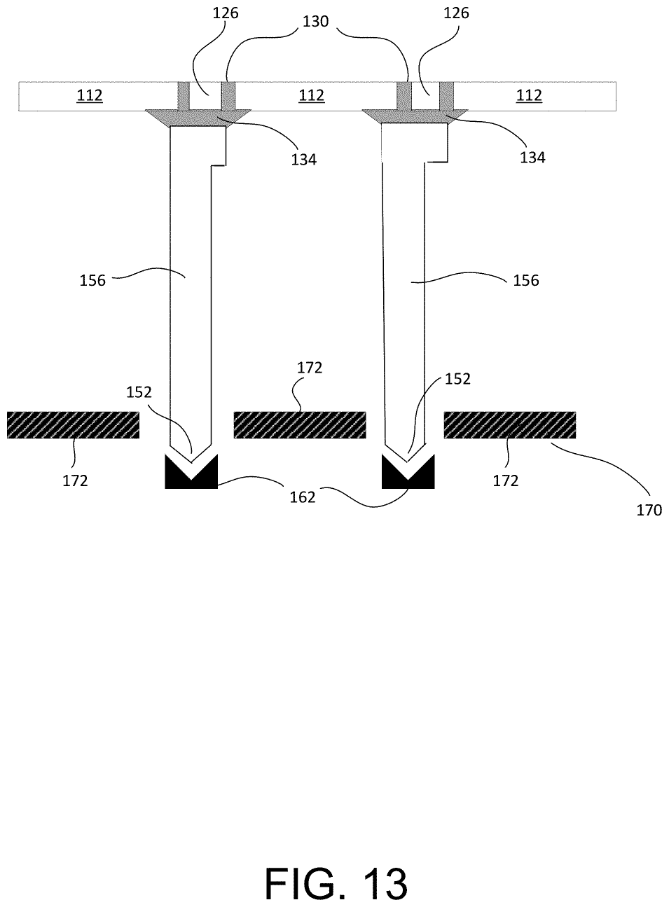

The template plate can be used as follows. During machining, the conductive block 150 may be first cut to a shallow depth (e.g. 150 um to 200 um) to yield the elongated protrusions 154. At this point, the elongated protrusions may be still relatively rigid since the aspect ratio is not very high. Next, the template plate 170 may be aligned and threaded onto the elongated protrusions 154, as shown in FIG. 11. The conductive block 150 may be further cut or milled back to yield the microwires 156, as shown in FIGS. 12 and 13.

Given that the template plate may be in the way of the wire of the wire-EDM, the wire may not be able to cut from the bottom side. To overcome this issue, an auto threader can be used with the wire-EDM. An auto threader is commonly used on an EDM if either the wire breaks or a new segment of the structure has to be cut. In the embodiments of the present disclosure, the auto threader can be used to thread the wire through a thin gap 151 left between the template plate 170 and the still uncut block 150. From there, the cutting of the conductive block 150 can continue towards the feedthrough device 140. In some embodiments, the template plate 170 may be repositioned out of the way of the wire between each cut to provide space for the wire.

FIG. 12 shows additional material of the conductive block 150 being removed to elongate the protrusions, and FIG. 13 shows microwires being formed and isolated from one another, in accordance with some embodiments. Referring to FIGS. 12 and 13, the conductive block 150 is milled back to a surface of the feedthrough device 140, to form a plurality of microwires 156. For example, wire-EDM can be used to cut slits into the conductive block 150 down to the base of the feedthrough device 140. The cutting process is self-terminating since the wire-EDM is unable to cut into the feedthrough device 140. For example, the cutting process can self-terminate at the insulating portion of the feedthrough device 140 which may comprise sapphire. The microwires 156 may be connected to the feedthrough device 140 via interconnects 134. The interconnects 134 may be the portion of the bonding material 132 that remains after the block cutting (e.g. wire-EDM) process. As shown in FIG. 13, the microwires 156 are spaced apart and separated from one another.

In some embodiments, after the microwires have been cut by the EDM, the microwires may be slightly etched further to make the microwires round and smooth along the length of the microwires, and to reduce the diameter of the microwires. In some cases, further electrosharpening of the tips of the microwires may be performed.

In some embodiments, the microwires 156 can be made wider towards the base (e.g. as shown in FIG. 14) so that there is more overlap between the conductive pads 126 of the feedthrough device 140 and the base of the microwires 156 (to increase hermeticity), or to make the microwires 156 mechanically more stable.

In some embodiments, one or more of the microwires 156 may be formed having a non-straight structure with one or more curved sections. In some cases, each of the microwires 156 may be formed having a non-straight structure with multiple curved sections along its length.

In some embodiments, designated weak zones may be formed along different sections of the microwires 156 during the wire-EDM process. These designated weak zones can allow the microwires 156 to fold or crumple in a controlled fashion when the insertion force of the microwires into brain tissue exceeds a predefined threshold.

In some embodiments, the microwires 156 may be coated with an insulating layer (not shown) along the length of the microwires. The insulating layer may be made of any appropriate material that is biocompatible and suitable for placement or insertion into neural matter. The insulating layer may be formed by a thermal drawing process, for example by drawing glass as a cladding over the microwires. The insulating layer may comprise glass, or any other suitable insulating materials such as silicone compounds (e.g., polydimethylsiloxane (PDMS), poly(methyl methacrylate) (PMMA), etc.), high temperature deposited oxides, medical-grade epoxies, organic polymer encapsulants, composite materials, and the like. In some embodiments, the insulating layer may comprise a plurality of insulating layers having one or more different material properties (e.g., dielectric constant, chemical reactivity/resistance, hardness, etc.).

FIG. 14 shows the backside of the feedthrough device 140 bonded to a chip 180 to form an active microprobe array 190, in accordance with some embodiments. The chip 180 may be an active device that is capable of recording voltage and/or generating current.

In some embodiments, the chip 180 may be a display driver chip. The chip may be a high performance readout integrated circuit (ROIC) chip that has been configured for adapted for neural recording. The chip may comprise a plurality of pixels/electrodes 182. In some embodiments, the chip may be a m×n pixel read out integrated circuit (ROIC) imaging chip with a total of m×n pixels/electrodes over an array area. The array area may be given by X1×Y1. In some embodiments, X1=Y1 such that the array has a square shape. In other embodiments, X1≠Y1 such that the array has a rectangular shape. The chip can be configured to acquire data at a rate of millions of pixels per second. The chip may have an adjustable gain current amplifier in each pixel circuit can be controlled by a series of input and output boards through a computer. The chip may be a multiplexed current readout chip with a gain amplifier in each unit cell or pixel.

The chip may include an m×n two-dimensional array of bond pads corresponding to the pixel array. Each of the bond pads may be individually addressable and configured to drive a pixel on a separate display (e.g., an LED or LCD-based display, not shown). The bond pads may be spaced apart by a pitch pₓ along an x-axis and by a pitch p_{y} along a y-axis. The pitches pₓ and p_{y} may be constant or variable. The pitches pₓ and p_{y} may be the same or different. In some embodiments, each of the pitches pₓ and p_{y} may be at least 10 μm, 50 μm, 100 μm, 200 μm, less than 10 μm, or greater than 200 μm. The pitch of the bond pads on the chip 180 may be customized based on the pitch of the conductive pads 126 on the feedthrough device 140.

In some embodiments, the plurality of pixels and the plurality of bonding pads may be provided in different array configurations. For example, in some instances (not shown), the plurality of pixels may be provided in a rectangular array, and the plurality of bonding pads may be provided in a non-rectangular array (e.g., a hexagonal array).

The bond pads may be formed of a regular shape or irregular shape, and may be formed having the same size or different sizes. Examples of regular shapes include rectangular, square, triangular, circular, elliptical, hexagonal, or any other known regular shapes. In some embodiments, the bond pads may include a mixed array of bond pads comprising bond pads of different shapes and/or sizes. The bond pads may be arranged in a regular pattern array. Alternatively, the bond pads may be arranged in an irregular pattern. The sizes of the bond pads may be determined by their dimensions, for example by their lengths, widths, heights, diameters, thicknesses, etc., depending on the shape and structural configuration in which the bond pads are formed. In some embodiments, all the bond pads may have the same height. In other embodiments, the bond pads may be formed having different heights. The bond pads may also have the same lateral dimensions (e.g., same diameter or length/width). Alternatively, the bond pads may have different lateral dimensions (e.g. different diameters or different lengths/widths).

The layout of the bond pads on the chip 180 may or may not directly match the geometry of the distal portion of the plurality of microwires 156. For example, a hexagonal pixel array may closely match a plurality of microwires that are arranged in a hexagonal closed-packed configuration. In contrast, a rectangular pixel array may match the plurality of microwires (arranged in a hexagonal closed-packed configuration) to a lesser degree compared to the hexagonal pixel array.

In some embodiments, the plurality of bond pads may occupy a significant fraction of the total surface area on the pixel region of the pixel side of the chip. For example, the bond pads may occupy 50%, 60%, 70%, 80%, or more than 80% of the total surface area on the pixel side of the chip.

In some embodiments, one or more pixels on the chip may be used as ground electrodes. Accordingly, those one or more pixels may be grounded, instead of being active pixels. One or more microwires may be connected to those “ground” pixels. Those wires may or may not include an insulating layer.

The feedthrough device 140 is configured to be attached (mechanically and electrically connected) to the chip 180 via the conductive pads 126. The mechanical/electrical coupling may be provided by a plurality of interconnects (not shown) formed at an interface between the bond pads of the chip 180 and the conductive pads 126 of the feedthrough device 140. The microwires 156 can be electrically connected to the plurality of bond pads of the chip 180 via the plurality of interconnects formed between the chip 180 and the feedthrough device 140. The interconnects allow the electrodes at the distal portion of the microwires to be in electrical communication with the integrated circuit elements on the chip 180, during monitoring and/or stimulation of brain activity.

Various interconnect structures and assembly methods thereof in association with the fabrication of a neural-interface probe are next described. In some embodiments, the interconnects between the chip 180 and the feedthrough device 140 may comprise solder bumps. The solder bumps may be formed of a low melting point metal or metallic alloy (e.g., In, or an In alloy). The solder bumps may have low levels of toxicity, and may not contain toxic metals such as Pb. The solder bumps can be screen printed, electroplated, or solder jetted. In some cases, solder balls may be physically placed onto the bond pads of the chip and reflowed to form the solder bumps. The solder bumps may be formed of any type of binary or ternary solder alloys. In some cases, the solder bumps may be formed of a lead-free solder such as SnAg, or a SnAg alloy (e.g., SnAgCu). In some embodiments, the interconnects may comprise a conductive polymer. In some embodiments, the interconnects may be surrounded by an underfill between the chip 180 and the feedthrough device 140. The underfill may comprise a biocompatible epoxy resin. The underfill can help to relieve thermomechanical stresses, by compensating for any mismatch in the coefficients of thermal expansion (CTEs) between the chip 180 and the feedthrough device 140.

In some cases, the epoxy resin may be dispensed on the feedthrough device 140 near an edge of the chip 180, and may flow through the gap between the feedthrough device 140 and the chip 180 via capillary action. The epoxy resin may be cured (hardened) to form the underfill. The curing process may include applying heat to the epoxy resin, for example by placing the assembly into a convection oven. Proper dispense and curing of the underfill can result in a smooth fillet between the edges of the chip 180 and the feedthrough device 140. The fillet can help to mitigate thermomechanical stresses that are induced.

In some embodiments, the chip 180 and the feedthrough device 140 may be electrically connected using an anisotropic conductive adhesive (ACA). The anisotropic conductive adhesive may comprise an epoxy resin containing a plurality of conductive particles. The anisotropic conductive adhesive may be biocompatible. The conductive particles can provide electrical connection in a z-direction, and can be used to form interconnects. The epoxy resin may serve as an underfill after curing. The conductive particles may be distributed spaced apart in the x-y direction such that they do not cause shorting between adjacent interconnects. In some embodiments, an anisotropic conductive film (ACF) may be used instead of an ACA. The ACF has similar properties to the ACA, except the ACF is laminated over the connection or conductive pads (instead of being dispensed in liquid form).

In some embodiments, the sharpened tips 152 of the microwires 156 may be electrochemically coated with a low-impedance coating, such as iridium oxide (or other transition-metal oxide, such as MnO₂, etc.), a conductive polymer (e.g., PEDOT, etc.), or a material promoting a high surface area (e.g. carbon nanotubes, platinum black, nanoparticle composites, and the like). The surface modification can decrease the interfacial electrical impedance between the exposed conductor core and brain tissue, thereby increasing the sensitivity of the neural-activity recording.

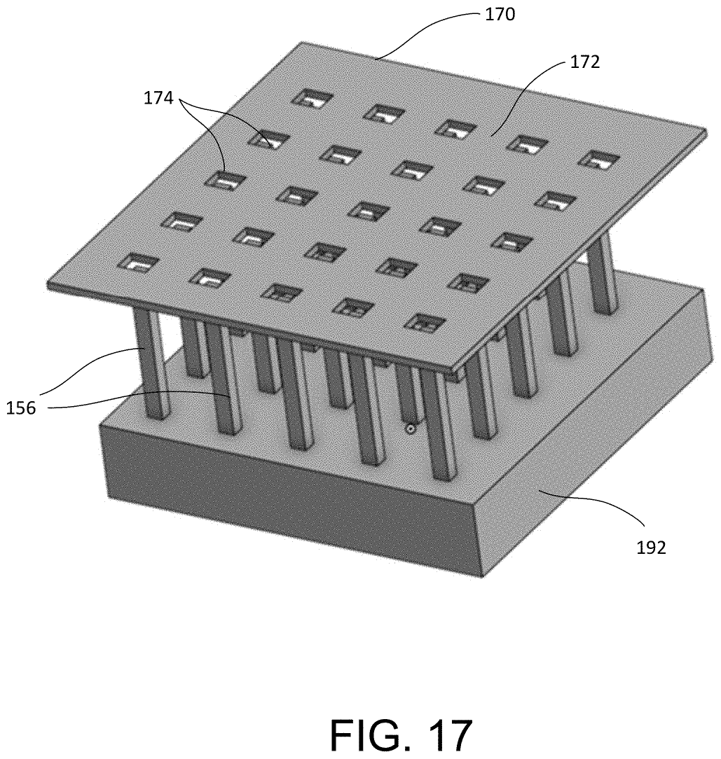

FIG. 15 shows the sharpened tips 152 of the microwires 156 being inserted into the brain of a subject using the template plate 170 for guiding the tips of the microwires, in accordance with some embodiments. As previously described, the template plate 170 comprises a plurality of holes 174 that negatively match the layout of the microwires 156. The template plate 170 can be integrated with the microprobe array 190, and can be translated (up or down) along the length of the microwires 156. The template plate 170 can be used to constrain and control the positions of the microwires 156, in particular when the template plate 170 is moved towards the tips 152 of the microwires 156. The template plate 170 can be helpful during manufacturing, to prevent excessive shaking of the elongated protrusions 154 during material removal in the conductive block 150. FIG. 16 shows a perspective view of the microprobe array of FIG. 14 without the template plate, and FIG. 17 shows a perspective view of the microprobe array of FIG. 14 with the template plate, in accordance with some embodiments. The microprobe array 190 may comprise a base portion 192 and the plurality of microwires 156 extending from the base portion. The base portion 192 may comprise the feedthrough device 140 and the chip 180, as shown in in FIG. 15. In some embodiments the base portion 192 may be enclosed in a housing (not shown). The housing can be configured to hermetically seal the chip 180 therein, and to prevent bodily or other fluid leakage onto the chip (which may cause damage to the chip). Referring to FIG. 17, the template plate 170 may aligned with the microwires 156, and can be configured to translate along the microwires when threaded through the microwires.

The plurality of microwires may be a wire bundle. The microwires may be configured to transmit electrical signals between the chip and neural matter within a brain. The microprobe array may comprise n number of wires, where n may be any integer greater than 1. For example, the microprobe array may comprise 100, 1000, 10000, or 1000000 wires, fewer than 100 wires, greater than 1000000 wires, or any number between the aforementioned ranges. In some embodiments, the microprobe array may further comprise at least one optical fiber (not shown) in addition to the microwires

The optical fiber may be configured to transmit light signals that enable imaging of the neural matter into which the microprobe array is inserted.

The template plate 170 can also help to constrain the microwires 156 during insertion into brain tissue, if the insertion method requires precise positioning of the tips of the microwires. The template plate 170 may be placed on a target region of the subject's brain. The template plate 170 can be used to guide the positioning of the microwires as the microwires are inserted into the target region. During insertion, the free ends of the microwires may spread out within the brain tissue such that the electrodes deploy in a three-dimensional arrangement over a target area. The target region may include a deep tissue region or a superficial tissue region. The superficial tissue region may, for example be a cortical region of the brain.

Neural-interface microprobe arrays of different lengths and other dimensions (width, etc.) may be used for different regions of the brain. The microprobe array described herein can be used to monitor and/or stimulate neural activity. In some embodiments, the microprobe probe may be inserted into a brain, such that the flexible distal portion of the microwires interfaces and is in contact with a target region of the neural matter. Neural activity in the target region can be monitored and/or stimulated via a plurality of electrical signals transmitted between the chip and the neural matter. The electrical signals may be transmitted through the plurality of microwires. In some embodiments, the electrical signals may be transmitted from the microprobe array to an external monitoring device via one or more wireless or wired communication channels.

In some embodiments, the implanted neural-interface microprobe array may be connected to the external world via a percutaneous wire. The percutaneous wire may be inserted through a patient's scalp. In other embodiments, the implanted neural-interface microprobe array may be connected to the external world via a wireless telemetry unit.

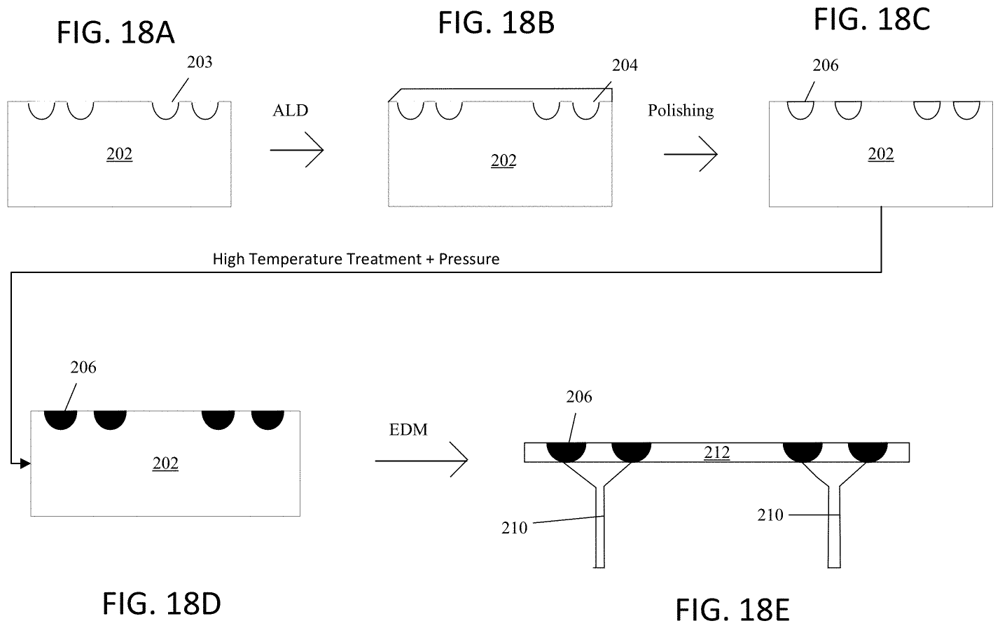

FIGS. 18A-18E illustrate a process for fabricating a microwire array, in accordance with some embodiments. The process shown in FIGS. 18A-18E can be used to form a feedthrough plate that exhibits a high level of hermeticity, and be substantially impermeable to fluids (e.g. water vapor) and ions. The feedthrough plate may correspond to the top part (206 and 212) of the structure shown in FIG. 18E.

Referring to FIG. 18A, a patterned block 202 is provided. The block may include a plurality of recesses 203 formed on a surface of the block. The term “block” or “substrate,” as used herein, generally refers to any substance to which other materials can be bonded, or upon which a layered structure can be deposited, or from which material be removed. The block 202 may be a conductive block. The block 202 may be made of a material comprising a transition metal. In some embodiments, the material may comprise a metal or metal alloy, for example platinum, iridium, niobium, chromium, scandium, titanium, vanadium, manganese, iron, cobalt, nickel, copper, zinc, yttrium, zirconium, gold, mercury, molybdenum, silver, tantalum, tungsten, aluminum, silicon, phosphorous, tin, an oxide of any of the preceding or any combination thereof. In other embodiments, the material may be a conductive ceramic such as TiN, conductive SiN, Indium tin oxide, etc. In some embodiments, the block 202 may comprise a platinum-iridium alloy.

Referring to FIG. 18A, the recesses 203 on the block 202 may include holes, trenches or cavities. The recesses may be formed using a variety of processes, for example laser drilling, etching (e.g. deep reactive ion etching), dry etch or wet etch processes, additive processes such as 3D printing, molding or sintering, etc. The block 202 may have a thickness ranging to 1 mm or greater. The recesses may be formed having a size (e.g. width or diameter) ranging from 5 um to 200 um. In some embodiments, the thickness of the block 202 may be about 500-2500 um, and the recesses 203 may have a size of about 100-500 um.

FIG. 18B shows a layer 204 deposited over the patterned surface of the block 202. The layer 204 may comprise a solid material such as a semiconductor or dielectric insulator. The material may be single crystalline, poly crystalline, or amorphous. Examples of materials in the layer 204 may include, for example, silicate glass, borosilicate glass, phosphate glass, aluminum oxide, sapphire, silicon, silicon dioxide, silicon nitride, silicon carbide, aluminum nitride, titanium nitride, titanium oxide, germanium, gallium arsenide, gallium nitride, indium phosphide, diamond, or synthetic diamond. In some embodiments, the layer 204 may comprise silicon, gallium, carbon, germanium, thallium, tellurium, selenium, or alloy or allotrope thereof, or an oxide or nitride thereof. In some embodiments, the layer 204 may be aluminum oxide (Al₂O₃). The layer 204 may be formed using any suitable deposition techniques. In some embodiments, the layer 204 may be formed using an atomic layer deposition (ALD) process or chemical vapor deposition or a powder that is sintered.

Next, referring to FIG. 18C, the layer 204 may be polished down to expose the surface of the block 202, and to form a plurality of insulative barriers 206. The layer 204 may be backgrind or polished using any bulk etch back or grinding/polishing/machining processes, for example chemical mechanical polishing (CMP).

Next, the structure in FIG. 18C is subject to a high temperature and pressure treatment process, to improve bonding between the insulative barriers 206 and the block 202. This process may also help increase the chemical stability of the block itself, such as driving out impurities.

FIG. 18E shows a portion of the block 202 in FIG. 18D being removed to form elongated protrusions 210. A portion of the block 202 can be removed by machining back the block to yield the high aspect ratio elongated protrusions 210. The machining process may include electric discharge machining (EDM), mechanical milling, LIGA, inductively coupled plasma (ICP) etching and the like.

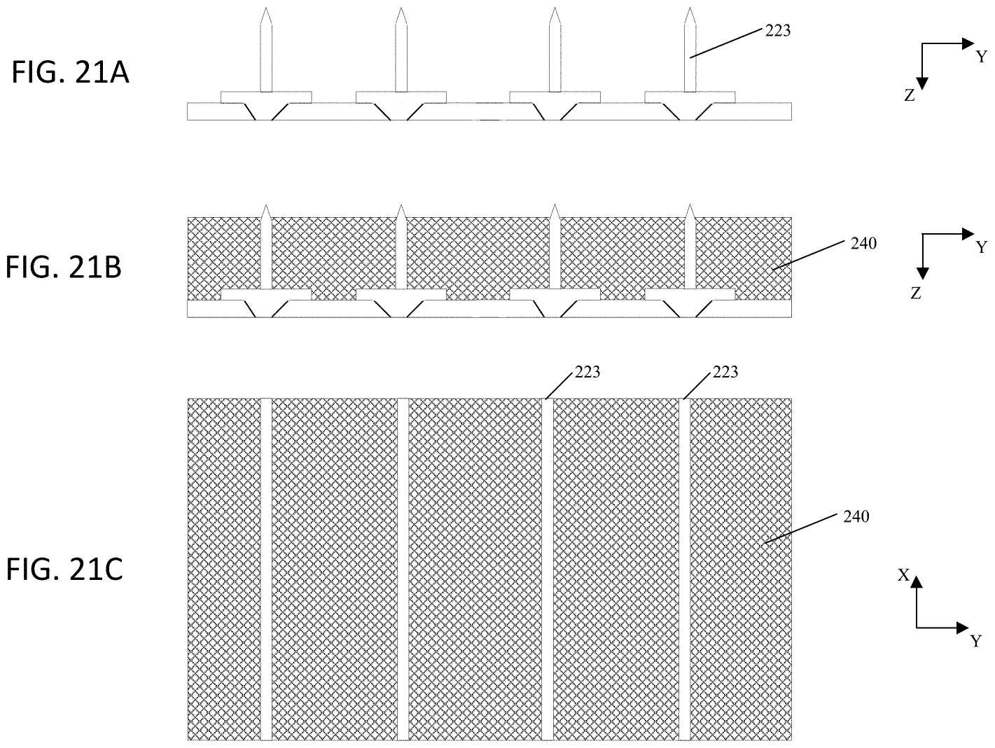

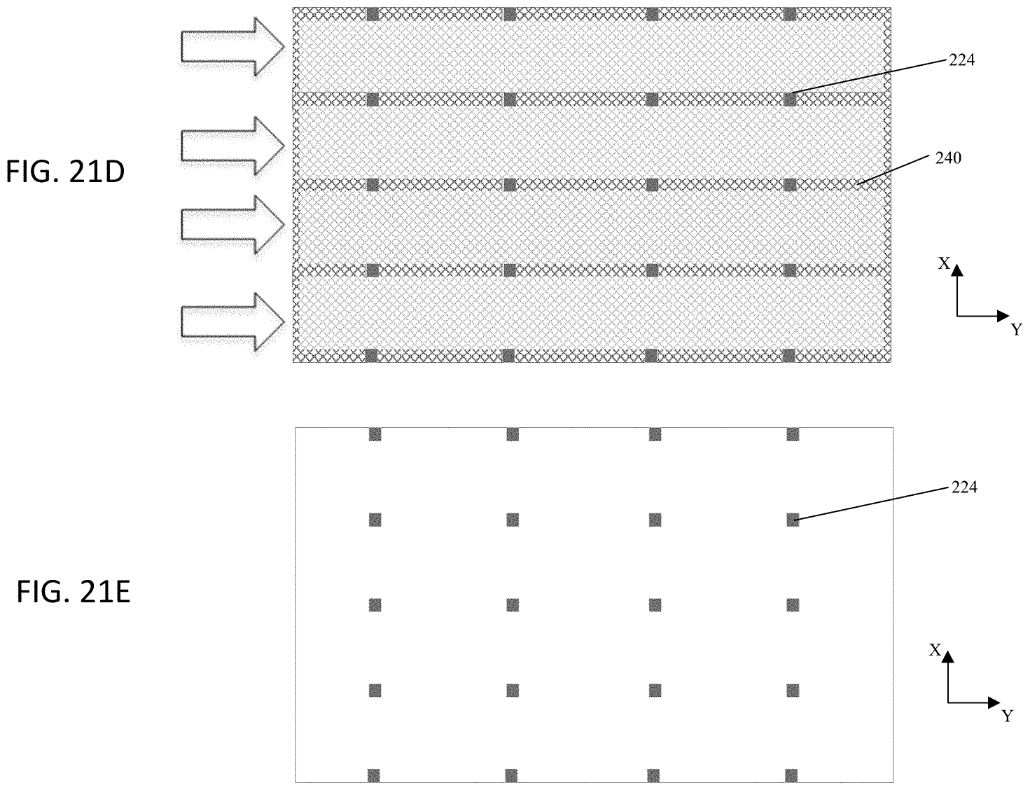

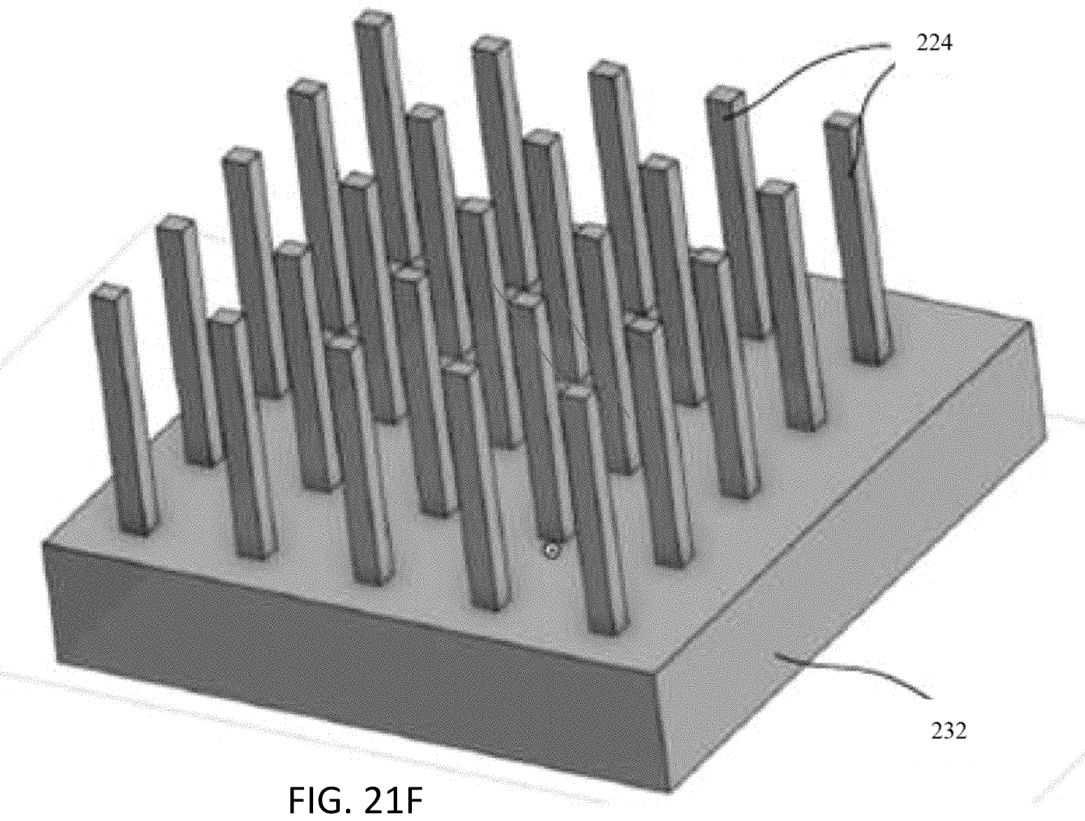

In some embodiments, the block 202 can be milled back using wire-EDM, which is a metal-working process whereby material is removed by an electro-thermal erosion mechanism. Wire-EDM can be used to fabricate metallic microelectrodes with high aspect ratio since there is no cutting force involved in material removal. Wire-EDM generally occurs in a controlled environment and utilizes precision actuation stages to enable positioning with micrometer accuracy. Wire-EDM can be used to cut slits/slots having a relatively small width (e.g. 30 um) and deep (e.g. on the order of several hundred microns) in a metal block. By cutting a set of parallel slits and then rotating the work piece (block) by an angle (e.g. 90 degrees) and repeating the cutting process, a microstructure array can be formed, for example shown in FIG. 21F. Although FIG. 21F shows an orthogonal microstructure array, the present disclosure is not limited thereto. For example, three sets of cuts at 60 degrees can be carried out to form a triangular patterned microstructure array.

The elongated protrusions 210 may include, for example microwires, fins, plates and the like. In the example of FIG. 18E, microwires 210 are formed and isolated from one another. The block 202 can be milled back to form the plurality of microwires 210. For example, wire-EDM can be used to cut slits into the block 202 down towards the base of the block. As shown in FIG. 18E, the microwires 210 are spaced apart and separated from one another, and electrically isolated from one another using insulative barriers 206.

In some embodiments, after the microwires have been cut by EDM, the microwires may be slightly etched further to make the microwires round and smooth along the length of the microwires, and to reduce the diameter of the microwires. In some cases, further electrosharpening of the tips of the microwires may be performed. The microwires may also be smoothed by using a second die-sink EDM process, where a preformed block with holes is aligned to the grid and used to do the final forming step of the microwires.

In some embodiments, the microwires 210 can be made wider towards the base (e.g. as shown in FIG. 18E) to increase hermeticity, or to make the microwires 210 mechanically more stable. The base may exhibit a high level of hermeticity, and be substantially impermeable to fluids (e.g. water vapor) and ions. In some embodiments, the base portion of the microwires may be chamfered, as shown in FIG. 18E.

In some embodiments (not shown), one or more of the microwires 210 may be formed having a non-straight structure with one or more curved sections. In some cases, each of the microwires 210 may be formed having a non-straight structure with multiple curved sections along its length.

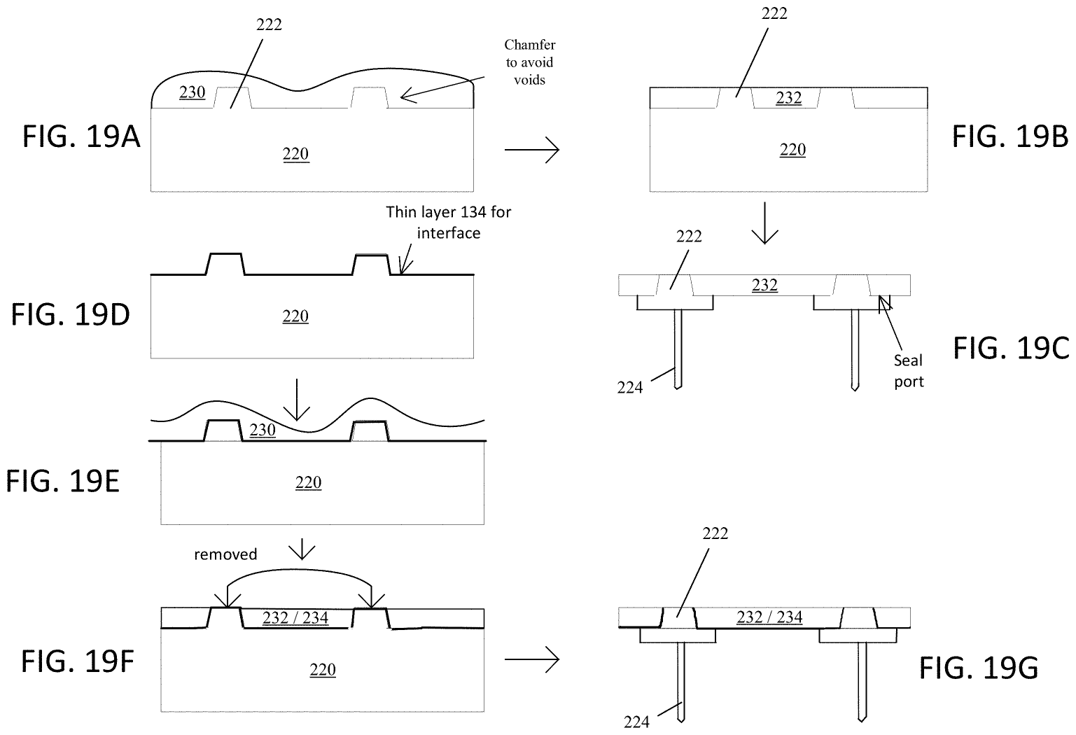

FIGS. 19A-19C illustrate an exemplary process for fabricating a device, in accordance with one embodiment. Referring to FIG. 19A, a patterned block 220 is provided. The block 220 may include a plurality of protrusions 222 formed on a surface of the block. The block 220 may be a conductive block. The block 220 may be made of a material comprising a transition metal. In some embodiments, the material may comprise a metal or metal alloy, for example platinum, iridium, niobium, chromium, scandium, titanium, vanadium, manganese, iron, cobalt, nickel, copper, zinc, yttrium, zirconium, gold, mercury, molybdenum, silver, tantalum, tungsten, aluminum, silicon, phosphorous, tin, an oxide of any of the preceding or any combination thereof. In other embodiments, the material may be a conductive ceramic such as TiN, conductive SiN, Indium tin oxide, etc. In some embodiments, the block 220 may comprise a platinum-iridium alloy.

The plurality of protrusions 222 may include pillars. The pillars 222 may extend or protrude from a surface of the block. The pillars may have a height ranging from 50 um to 1 mm, and a width (or diameter) ranging from 2 um to 90 um. In some embodiments, the pillars (or microwires) may have a height of about 150 um, and a width (or diameter) of about 10 um. In some preferred embodiments, the block 220 may be a platinum-iridium alloy block, and the pillars may be formed by etching the block. In some instances, the pillars may be formed by electric discharge machining. In some embodiments, the edges of the pillars may be chamfered to reduce or avoid voids when a layer 230 is deposited over the pillars and surface of the block, for example as shown in FIG. 19A. The layer 230 may comprise a solid material such as a semiconductor. The material 230 may be single crystalline, poly crystalline, or amorphous. Examples of materials in the layer 230 may include, for example, aluminum oxide, sapphire, silicon, silicon dioxide, silicon carbide, aluminum nitride, germanium, gallium arsenide, gallium nitride, indium phosphide, diamond, or synthetic diamond. In some embodiments, the layer 230 may comprise silicon, gallium, carbon, germanium, thallium, tellurium, selenium, or alloy or allotrope thereof, or an oxide or nitride thereof. In some embodiments, the layer 230 may include aluminum oxide (Al₂O₃). The layer 230 may be formed using any suitable deposition techniques, for example chemical vapor deposition (CVD), sputtering, evaporation, and the like.

Next, referring to FIG. 19B, the layer 230 may be polished down to expose the top portion of the pillars 222, and to form a plurality of insulative barriers 232. The layer 230 may be backgrind or polished using any bulk etch back or grinding/polishing/machining processes, for example chemical mechanical polishing (CMP).

Next, referring to FIG. 19C, a portion of the block 220 may be removed to form elongated protrusions 224. A portion of the block 220 can be removed by machining back the block to yield the high aspect ratio elongated protrusions 224. The machining process may include electric discharge machining (EDM), mechanical milling, LIGA, inductively coupled plasma (ICP) etching and the like. For example, the elongated protrusions 224 may be formed using wire EDM similar to the process described earlier in FIG. 18E.

The elongated protrusions 224 may include microwires, fins or plates. FIG. 19C shows microwires 224 being formed and isolated from one another. The block 220 can be milled back to form a plurality of microwires 224. For example, wire-EDM can be used to cut slits into the block 220 down towards the base of the block. As shown in FIG. 19C, the microwires 224 are spaced apart and separated from one another, and electrically isolated from one another using insulative barriers 232.

In some embodiments, after the microwires have been cut by the EDM, the microwires may be slightly etched further to make the microwires round and smooth along the length of the microwires, and to reduce the diameter of the microwires. In some cases, further electrosharpening of the tips of the microwires may be performed. The microwires may also be smoothed by using a second die-sink EDM process, where a preformed block with holes is aligned to the grid and used to do the final forming step of the microwires.

In some embodiments, the microwires 224 can be made wider towards the base (e.g. as shown in FIG. 19C) to increase hermeticity, or to make the microwires 224 mechanically more stable. For example, the widened base portion of the microwires may act as a seal port, as shown in FIG. 19C. Hermetic sealing can help to prevent fluids from leaking into and entering the chip which can cause electrical failures. The base may exhibit a high level of hermeticity, and be substantially impermeable to fluids (e.g. water vapor) and ions.

FIGS. 19D-19G illustrate an exemplary process for fabricating a microstructure array, in accordance with another embodiment. The embodiment of FIGS. 19D-19G is similar to the embodiment of FIGS. 19A-19C, except a base layer 234 is first deposited over the surface of the pillars 222 and the block 220 prior to depositing the layer 230. The base layer 234 may be a thin layer that is deposited using, for example, atomic layer deposition (ALD), chemical vapor deposition or physical vapor deposition (i.e. sputtering). The base layer 234 can serve as an adhesion layer, and can promote interfacial adhesion between the layer 230 and the block 220. The base layer 234 may be made of a suitable material. For example, the material may include a semiconductor or a dielectric insulator. The material may be single crystalline, poly crystalline, or amorphous. Examples of materials may include, for example, silicate glass, borosilicate glass, phosphate glass, aluminum oxide, sapphire, silicon, silicon dioxide, silicon nitride, silicon carbide, aluminum nitride, titanium nitride, titanium oxide, germanium, gallium arsenide, gallium nitride, indium phosphide, diamond, or synthetic diamond. In some embodiments, the material may comprise silicon, gallium, carbon, germanium, thallium, tellurium, selenium, or alloy or allotrope thereof, or an oxide or nitride thereof. In some embodiments, the material may be aluminum oxide (Al₂O₃). The base layer 234 may be formed using any suitable deposition techniques. In some embodiments, the base layer 234 may be formed using an atomic layer deposition (ALD) process or chemical vapor deposition or a powder that is sintered. The material may also include glasses such as borosilicate, phosphate glass, silica, quartz, nitrides, etc. In some embodiments, the deposition temperature or conditions may be increased to form epitaxial growth of the base layer 234.

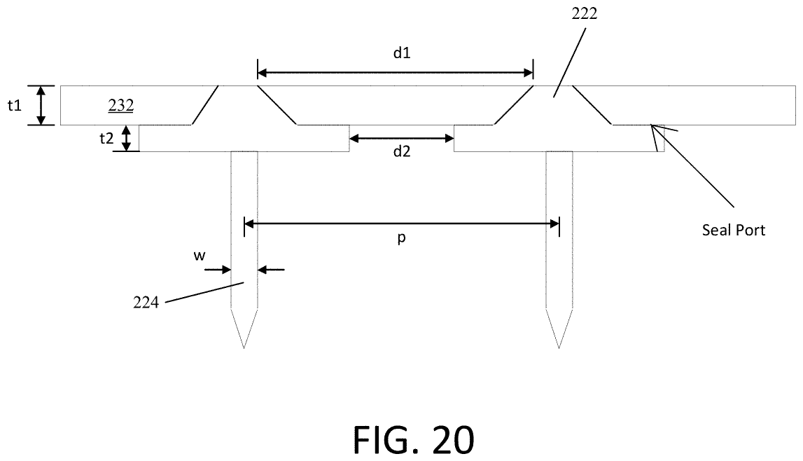

FIG. 20 illustrates a cross section of a microstructure array, in accordance with some embodiments. The microstructure array may correspond to the example shown in FIG. 19C. The insulative barriers 232 may have a thickness t1 ranging from about 50 um to about 400 um. A base portion of the pillars 222 may have a thickness t2 ranging from about 25 um to about 100 um. A top portion of adjacent pillars 222 may be spaced apart by a distance d1 ranging from about 40 um to about 250 um. The base portion of adjacent pillars 222 may be spaced apart by a distance d2 ranging from about 10 um to about 150 um. The microwires 224 may have a pitch spacing p ranging from about 50 um to about 250 um. Each microwire 224 may have a width w ranging from about 10 um to about 60 um.

FIGS. 21A-21F illustrate a process for fabricating a microstructure array, in accordance with some embodiments. FIG. 21A shows a plurality of extended protrusions 223 that are formed by machining back a block. The extended protrusions 223 may include a plurality of fins or fin-like structures. The machining process may include electric discharge machining (EDM), mechanical milling, LIGA, inductively coupled plasma (ICP) etching and the like. The extended protrusions 223 may be formed by cutting a set of parallel slits in the block in a first direction (e.g. X-axis), to a depth on the order of several hundred microns.

Next, a support material 240 may be deposited into the spaces between the extended protrusions 223, as shown in FIG. 21B (sectional view) and FIG. 21C (planar view). The support material 240 can be used provide support and structural rigidity to the extended protrusions 223 as they undergo machining in a second direction (e.g. Y-axis). This can reduce vibration and unwanted movements during the EDM process in the second direction, which may affect the geometric accuracy of the microstructures. In addition, the support material 240 can help to absorb or dissipate machining heat, thereby reducing the risk of the microwires deforming or breaking due to excessive absorption of Joule heat. The support material may also improve the quality of the machining surface finish.

The support material 240 may be any material that has a relatively low melting point, and can be reflowed or dispensed into the spaces between the extended protrusions 223 to form a monolithic solid block-shaped object. For example, the support material 240 may include gold, indium, tin, or a solder alloy. In some embodiments, the reflow of the support material 240 may be performed in a low pressure environment under vacuum.

The support material 240 may be made of any appropriate material that is biocompatible. In some embodiments, the biocompatible material may comprise gold, copper, platinum, silver, or any metallic alloy. In some embodiments, the support material 240 may comprise a semiconductor, a conductive polymer, or a conductive composite material. In some embodiments, the support material 240 may comprise an insulating material such as silicone compounds (e.g., polydimethylsiloxane (PDMS), poly(methyl methacrylate) (PMMA), etc.), medical-grade epoxies, organic polymer encapsulants, composite materials, and the like. In some embodiments, the support material 240 may be chosen such that the material is capable of dissolving within a subject's body, and thus disappears after the support material 240 has served its function of stabilizing the wires during the EDM process.

In some other embodiments, the support material 240 need not be biocompatible. In some embodiments, the support material 240 may be conductive. In some alternative embodiments, the support material 240 may be non-conductive. The support material 240 may also be deposited using low-stress methods such as electroplating.

FIG. 21D shows the machining in the second direction (e.g. along the Y-axis) to form a plurality of stand-alone microwires 224. As described above, the support material 240 can provide support during the subsequent machining, and prevent the microstructures from yielding or buckling during the EDM process.

After the microwires 224 have been formed, the support material 240 is removed, resulting in the structure shown in FIG. 21E (planar view) and FIG. 21F (perspective view). The support material 240 may be etched using a preferential etching process that removes the support material 240 with little impact on the microwires 224 and the substrate on which the microwires are supported.

In the example shown in FIGS. 21A-21F, although the cuts are made in two orthogonal directions (X and Y axes), the present disclosure is not limited thereto. For example, the cuts may be made in three or more different directions, at a variety of different angles (e.g. 45 degrees, 60 degrees, etc.).

In some embodiments, sharpened tips may be formed at the distal ends of the microwires. The sharpened tips may be formed by etching the conductive block along a set of predefined planes, by ion beam material removal, by laser milling, by electric discharge machining, or by mechanical milling. The sharpened tips may have a beveled cut surface. In some embodiments, the beveled cut surface may have an angle of about 5 degrees, 10 degrees, 20 degrees, 30 degrees, 40 degrees, 50 degrees, 60 degrees, 70 degrees, 80 degrees, 90 degrees, or any angle within a range between any two of the preceding values. The sharpened tips can help to facilitate insertion into brain tissue.

In some embodiments, an electrochemical modification process can be used to form sharpened tips. Electrochemical modification can be carried out in an electrically conductive bath. In the case of subtractive electrochemical modification, the bath may contain reactive elements such as sodium or potassium hydroxide, at a concentration above 0.01M and below 10M. The bath may also contain acids such as sulfuric or phosphoric acid. The subtractive process may be performed under an applied voltage at a metal core, which may typically be positive. The specific formulation used in the bath can vary depending on the selected material, as known to those skilled in the art.

Subtractive modification may result in two types of general geometries: (1) smooth and flat material removal (often called electropolishing), or (2) the sharpening of the wire to an apex (called electrosharpening). The geometries may depend on factors such as mass transport of chemical reagents to the material surface, applied potential at the electrode surface, solution composition, temperature, applied forces to the electrode during the process, and the like.

The additive process may be carried out using a solution that contains the dissolved ions of the material. An electric potential may be applied to the electrode which is undergoing modification, but this may not be necessary. When a potential is applied, it may typically be negative, which causes the dissolved ions to reduce onto the surface of the material as a solid. When a potential is not applied to the electrode undergoing modification, a reducing agent may typically be included in the solution to enable the application of the material coating. Growth of material at the surface of the electrode may take on different forms and morphologies depending on the deposition conditions, including reagent chemistry, chemical mass transport, temperature, electric potential applied to the electrode, and the like. In some embodiments, deposition of the additive solution may be carried out by local deposition using a pipette or micropipette or any other suitable tool that is capable of dispensing volumes from 1 um3 to 10000 um3. The additive solution may also be applied via the use of surface tension, for example by dipping the array into a solution and withdrawing it so that the surface tension of the liquid holds it in place. The additive solution may also be added via condensation, being nucleated at the tip of the wire via an evaporative or Ostwald ripening process. The process may also be carried out by the formation of a mask layer on top of the electrode, after which material can be deposited by a sputtering, evaporation, or electrochemical process.

In some embodiments, the sharpened tips of the microwires may be electrochemically coated with a low-impedance coating, such as iridium oxide (or other transition-metal oxide, such as MnO₂, etc.), a conductive polymer (e.g., PEDOT, etc.), or a material promoting a high surface area (e.g. carbon nanotubes, platinum black, nanoparticle composites, and the like). The surface modification can decrease the interfacial electrical impedance between the exposed conductor core and brain tissue, thereby increasing the sensitivity of the neural-activity recording.

In some embodiments, designated weak zones may be formed along different sections of the microwires during the wire-EDM process. These designated weak zones can allow the microwires to fold or crumple in a controlled fashion when the insertion force of the microwires into brain tissue exceeds a predefined threshold.

In some embodiments, the microwires may be coated with an insulating layer (not shown) along the length of the microwires. The insulating layer may be made of any appropriate material that is biocompatible and suitable for placement or insertion into neural matter. The insulating layer may be formed by a thermal drawing process, for example by drawing glass as a cladding over the microwires. The insulating layer may comprise glass, or any other suitable insulating materials such as silicone compounds (e.g., polydimethylsiloxane (PDMS), poly(methyl methacrylate) (PMMA), etc.), high temperature deposited oxides, medical-grade epoxies, organic polymer encapsulants, composite materials, and the like. In some embodiments, the insulating layer may comprise a plurality of insulating layers having one or more different material properties (e.g., dielectric constant, chemical reactivity/resistance, hardness, etc.).

Any of the devices herein may be bonded to a chip to form an active microprobe array. The chip may be an active device that is capable of recording voltage and/or generating current.

In some embodiments, the chip may be a display driver chip. The chip may be a high performance readout integrated circuit (ROIC) chip that has been configured for adapted for neural recording. The chip may comprise a plurality of pixels/electrodes. In some embodiments, the chip may be a m×n pixel read out integrated circuit (ROIC) imaging chip with a total of m×n pixels/electrodes over an array area. The array area may be given by X1×Y1. In some embodiments, X1=Y1 such that the array has a square shape. In other embodiments, X1≠Y1 such that the array has a rectangular shape. The chip can be configured to acquire data at a rate of millions of pixels per second. The chip may have an adjustable gain current amplifier in each pixel circuit can be controlled by a series of input and output boards through a computer. The chip may be a multiplexed current readout chip with a gain amplifier in each unit cell or pixel.

The chip may include an m×n two-dimensional array of bond pads corresponding to the pixel array. Each of the bond pads may be individually addressable and configured to drive a pixel on a separate display (e.g., an LED or LCD-based display, not shown). The bond pads may be spaced apart by a pitch px along an x-axis and by a pitch py along a y-axis. The pitches px and py may be constant or variable. The pitches px and py may be the same or different. In some embodiments, each of the pitches px and py may be at least 10 μm, 50 μm, 100 μm, 200 μm, less than 10 μm, or greater than 200 μm. The pitch of the bond pads on the chip may be customized based on the pitch of the conductive pads on the device.

The device herein is configured to be attached (mechanically and electrically connected) to a chip via conductive pads. The mechanical/electrical coupling may be provided by a plurality of interconnects (not shown) formed at an interface between the bond pads of the chip, and the conductive pads of the device. The microwires can be electrically connected to the plurality of bond pads of the chip via the plurality of interconnects formed between the chip and the device. The interconnects allow the electrodes at the distal portion of the microwires to be in electrical communication with the integrated circuit elements on the chip, during monitoring and/or stimulation of brain activity.

Neural-interface microprobe arrays of different lengths and other dimensions (width, etc.) may be used for different regions of the brain. The microprobe array described herein can be used to monitor and/or stimulate neural activity. In some embodiments, the microprobe probe may be inserted into a brain, such that the flexible distal portion of the microwires interfaces and is in contact with a target region of the neural matter. Neural activity in the target region can be monitored and/or stimulated via a plurality of electrical signals transmitted between the chip and the neural matter. The electrical signals may be transmitted through the plurality of microwires. In some embodiments, the electrical signals may be transmitted from the microprobe array to an external monitoring device via one or more wireless or wired communication channels.

In some embodiments, the implanted neural-interface microprobe array may be connected to the external world via a percutaneous wire. The percutaneous wire may be inserted through a patient's scalp. In other embodiments, the implanted neural-interface microprobe array may be connected to the external world via a wireless telemetry unit.

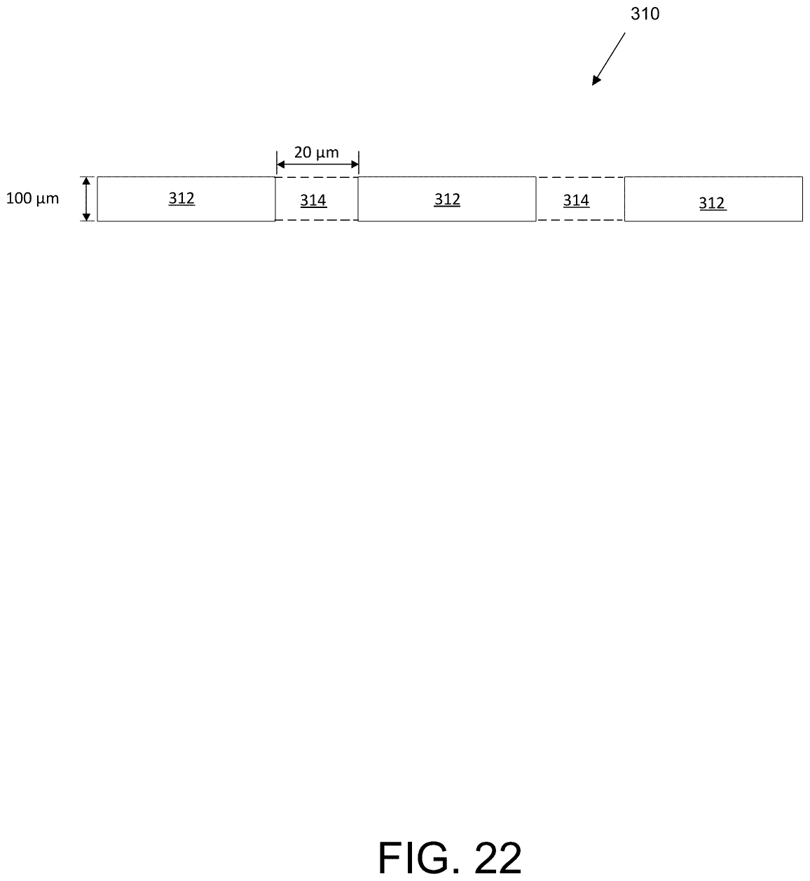

FIG. 22 illustrates a patterned substrate 310 in accordance with some embodiments. The term “substrate,” as used herein, generally refers to any substance to which other materials can be bonded, or upon which a layered structure can be deposited. The substrate 310 may comprise a solid material such as a semiconductor or an insulator. The substrate material may be single crystalline, poly crystalline, or amorphous. Substrate materials may comprise, for example, ceramic, sapphire, silicon, silicon dioxide, silicon carbide, aluminum oxide, aluminum nitride, germanium, gallium arsenide, gallium nitride, indium phosphide, diamond, or synthetic diamond. In some embodiments, the substrate can be a ceramic substrate. In some embodiments, substrate materials may comprise silicon, gallium, carbon, germanium, arsenic, thallium, cadmium, tellurium, selenium, or alloy or allotrope thereof, or an oxide or nitride thereof. In some embodiments, the substrate may include one or more chemical dopants, such as nitrogen, phosphorous, boron or indium.

In some embodiment, the substrate may have a thickness of about 5 micrometer (μm) to about 1 millimeter (mm). In some embodiment, the thickness of the substrate may be about 5 μm to about 50 μm, about 5 μm to about 100 μm, about 5 μm to about 200 μm, about 5 μm to about 300 μm, about 5 μm to about 400 μm, about 5 μm to about 500 μm, about 5 μm to about 600 μm, about 5 μm to about 700 μm, about 5 μm to about 800 μm, about 5 μm to about 900 μm, about 5 μm to about 1 mm, about 50 μm to about 100 μm, about 50 μm to about 200 μm, about 50 μm to about 300 μm, about 50 μm to about 400 μm, about 50 μm to about 500 μm, about 50 μm to about 600 μm, about 50 μm to about 700 μm, about 50 μm to about 800 μm, about 50 μm to about 900 μm, about 50 μm to about 1 mm, about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1 mm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1 mm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1 mm, about 400 μm to about 500 μm, about 400 μm to about 600 μm, about 400 μm to about 700 μm, about 400 μm to about 800 μm, about 400 μm to about 900 μm, about 400 μm to about 1 mm, about 500 μm to about 600 μm, about 500 μm to about 700 μm, about 500 μm to about 800 μm, about 500 μm to about 900 μm, about 500 μm to about 1 mm, about 600 μm to about 700 μm, about 600 μm to about 800 μm, about 600 μm to about 900 μm, about 600 μm to about 1 mm, about 700 μm to about 800 μm, about 700 μm to about 900 μm, about 700 μm to about 1 mm, about 800 μm to about 900 μm, about 800 μm to about 1 mm, or about 900 μm to about 1 mm. In some embodiment, the thickness of the substrate may be about 5 μm, about 50 μm, about 100 μm, about 200 μm, about 300 μm, about 400 μm, about 500 μm, about 600 μm, about 700 μm, about 800 μm, about 900 μm, or about 1 mm. In some embodiment, the thickness of the substrate may be at least about 5 μm, about 50 μm, about 100 μm, about 200 μm, about 300 μm, about 400 μm, about 500 μm, about 600 μm, about 700 μm, about 800 μm, about 900 μm, about 1 mm or more. In some embodiment, the thickness of the substrate may be at most about 1 mm, about 900 μm, about 800 μm, about 700 μm, about 600 μm, about 500 μm, about 400 μm, about 300 μm, about 200 μm, about 100 μm, about 50 μm, about 5 μm, or less.

Referring to FIG. 22, the patterned substrate 300 may comprise a plurality of patterned portions 312 and holes 314 (e.g., feedthroughs). The holes may be through-holes (e.g., feedthroughs). The holes may be formed using a variety of processes, for example laser drilling, etching (e.g. deep reactive ion etching), dry etch or wet etch processes, additive processes such as 3D printing, molding or sintering, etc. In some preferred embodiments, the patterned substrate 310 may be made of sapphire, and the holes may be formed by laser drilling through an unpatterned sapphire substrate. The patterned substrate 310 may be formed having a thickness ranging from 5 um to 1 mm. The holes 314 may be formed having a size (e.g. width or diameter) ranging from 5 um to 100 um. In some embodiments, the thickness of the patterned substrate 310 may be about 100 um, and the holes may have a size of about 20 um.

In some embodiment, the diameter of the feedthrough holes 314 may be between about 10 micrometers (μm) to about 300 μm. In some embodiment, the diameter of the feedthrough holes may be about 10 μm to about 20 μm, about 10 μm to about 25 μm, about 10 μm to about 50 μm, about 10 μm to about 100 μm, about 10 μm to about 150 μm, about 10 μm to about 200 μm, about 10 μm to about 250 μm, about 10 μm to about 300 μm, about 20 μm to about 25 μm, about 20 μm to about 50 μm, about 20 μm to about 100 μm, about 20 μm to about 150 μm, about 20 μm to about 200 μm, about 20 μm to about 250 μm, about 20 μm to about 300 μm, about 25 μm to about 50 μm, about 25 μm to about 100 μm, about 25 μm to about 150 μm, about 25 μm to about 200 μm, about 25 μm to about 250 μm, about 25 μm to about 300 μm, about 50 μm to about 100 μm, about 50 μm to about 150 μm, about 50 μm to about 200 μm, about 50 μm to about 250 μm, about 50 μm to about 300 μm, about 100 μm to about 150 μm, about 100 μm to about 200 μm, about 100 μm to about 250 μm, about 100 μm to about 300 μm, about 150 μm to about 200 μm, about 150 μm to about 250 μm, about 150 μm to about 300 μm, about 200 μm to about 250 μm, about 200 μm to about 300 μm, or about 250 μm to about 300 μm. In some embodiment, the diameter of the feedthrough holes may be about 10 μm, about 20 μm, about 25 μm, about 50 μm, about 100 μm, about 150 μm, about 200 μm, about 250 μm, or about 300 μm. In some embodiment, the diameter of the feedthrough holes may be at least about 10 μm, about 20 μm, about 25 μm, about 50 μm, about 100 μm, about 150 μm, about 200 μm, about 250 μm, or more. In some embodiment, the diameter of the feedthrough holes may be at most about 300 μm, about 250 μm, about 200 μm, about 150 μm, about 100 μm, about 50 μm, about 25 μm, about 20 μm, about 10 μm, or less.

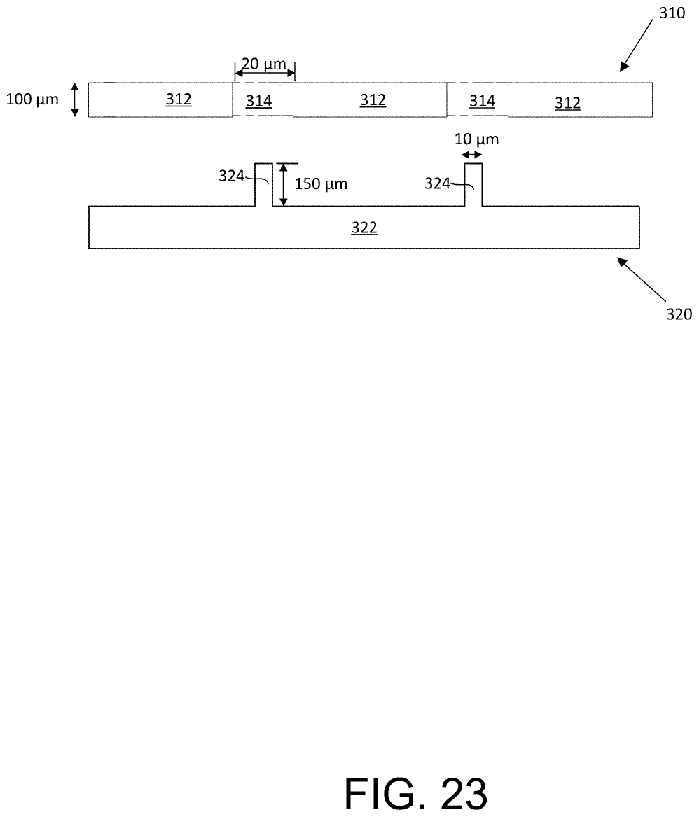

FIG. 23 shows a patterned matching block 320 in proximity and aligned with the patterned substrate 310 of FIG. 22, in accordance with some embodiments. In some embodiments, the patterned matching block 320 may be made of a material comprising a transition metal. In some embodiments, the material may comprise niobium, chromium, scandium, titanium, vanadium, manganese, iron, cobalt, nickel, copper, zinc, yttrium, zirconium, platinum, gold, mercury, iridium, molybdenum, silver, tantalum, tungsten, aluminum, silicon, phosphorous, tin, an oxide of any of the preceding or any combination thereof. In other embodiments, the material may be a conductive ceramic such as TiN, conductive SiN, Indium tin oxide, etc. The patterned matching block 320 may be formed having a pattern that matches with the patterned substrate 310. The patterned matching block 320 may comprise a base portion 322, and a plurality of pins (or pillars) (or microwires) 324 on the base portion. The pins 324 may extend or protrude from a surface of the base portion. The pins may have a height ranging from 50 um to 1 mm, and a width (or diameter) ranging from 2 um to 90 um. In some embodiments, the pins may have a height of about 150 um, and a width (or diameter) of about 10 micrometers (μm), to about 60 μm. In some preferred embodiments, the patterned matching block 320 may be made of niobium, and the pins may be formed by etching a niobium block using a mask. In some instances, the pins may be formed by electric discharge machining.

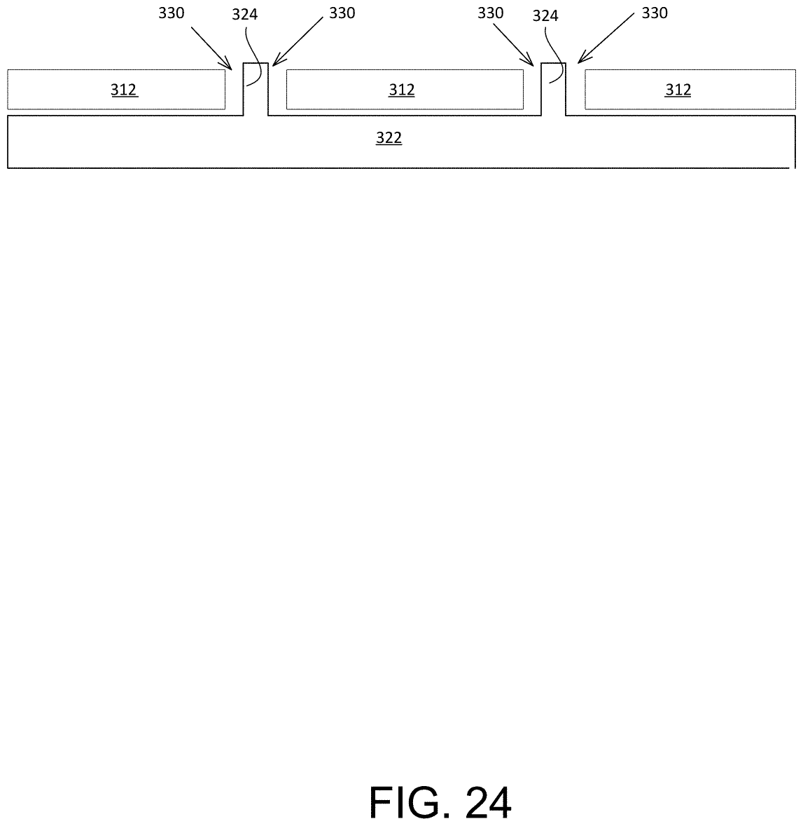

FIG. 24 shows the bonding of the patterned matching block 320 with the patterned substrate 310, in accordance with some embodiments. When the pins of the patterned matching block 320 are aligned with the holes of the patterned substrate 310, the patterned matching block 320 and the patterned substrate 310 are brought into proximity with each other such that the pins 324 are located in the holes 314, as shown in FIG. 24. Next, the patterned matching block 320 and the patterned substrate 310 are bonded to each other, by reflowing a bonding material 330 into the gaps between the patterned matching block 320 and the patterned substrate 310. The bonding material may comprise a conductive material that has a relatively low melting point, and that is capable of filling the gaps completely. In some embodiments, the bonding material may comprise gold. In some cases, the reflow of the bonding material and the bonding process may be performed in a low pressure environment under vacuum.

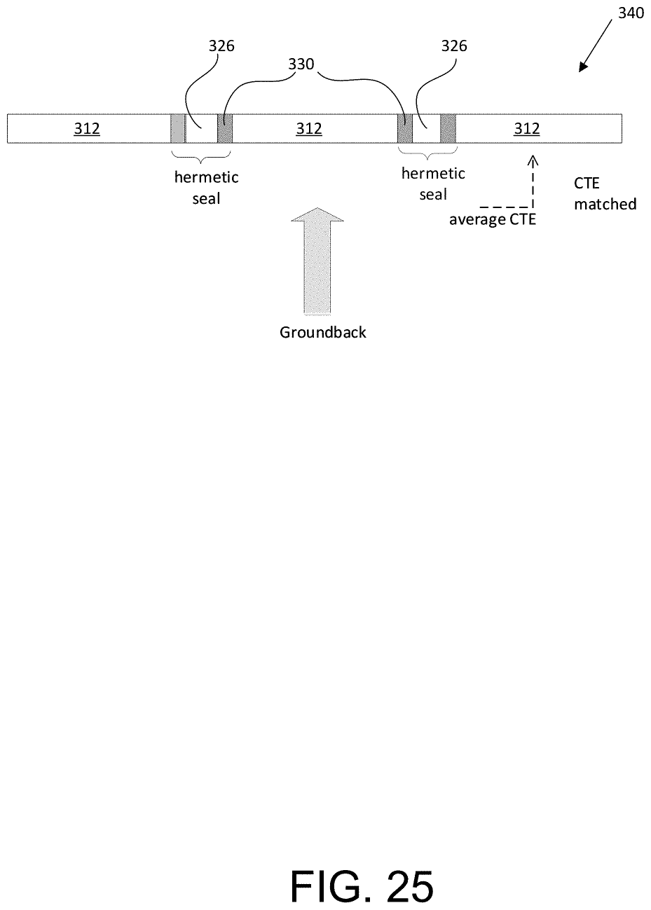

FIG. 25 shows a feedthrough device 340 that is formed after backgrinding of the bonded assembly of FIG. 24, in accordance with some embodiments. The backgrinding may be performed on one side, or both opposite sides of the bonded assembly. The backgrinding may be performed using any bulk etch back or grinding/polishing/machining processes, for example chemical mechanical polishing (CMP). The backgrinding also enables planarization on both surfaces of the feedthrough device 340. In some embodiments, the feedthrough device 340 may have a thickness of about or less than 100 um.

Referring to FIG. 25, the feedthrough device 340 may comprise a plurality of conductive pads 326 (e.g., conductive feedthroughs) spaced apart and separated from one another by insulating portions. The conductive pads 326 may include the backgrinded remaining portion of the pins 324. The insulating portions may comprise the patterned portions 312 of the patterned substrate 310. In some embodiments, the patterned portions 312 may comprise sapphire. In some embodiments, the conductive pads 326 may comprise niobium which is bonded to the patterned portions 312 via the bonding material 330 (e.g. gold). The bonding material 330 is configured to form a hermetic seal that prevents any fluid or ions from flowing through the feedthrough device 340. The feedthrough device 340 may be bonded to a chip. The hermetic sealing can help to prevent fluids from leaking into and entering the chip which can cause electrical failures. The bonding material 330 is also configured to reduce a coefficient of thermal expansion (CTE) mismatch between the bonded conductive pads 326 and the patterned portions 312, which are made of different materials and may be subject to thermomechanical stresses. This may be achieved by selecting dimensions such that the weighted average CTE of the materials 326 and 330 match the CTE of material 312.

It should be appreciated that there may be other methods for forming the feedthrough device 340. In some embodiments, a feedthrough device may be formed by creating through holes or through vias in a substrate, or machining holes in a substrate. Next, the conductive pads may be formed by filling the through vias or holes with a conductive material. The conductive material may be electroplated to form the conductive pads. In some instances, the conductive material (e.g. metal particles) may be reflowed to form the conductive pads.

In some embodiment, the width of the conductive feedthrough 326 may be between about 10 micrometers (μm) to about 300 μm. In some embodiment, the width of the conductive feedthrough may be about 10 μm to about 20 μm, about 10 μm to about 25 μm, about 10 μm to about 50 μm, about 10 μm to about 100 μm, about 10 μm to about 150 μm, about 10 μm to about 200 μm, about 10 μm to about 250 μm, about 10 μm to about 300 μm, about 20 μm to about 25 μm, about 20 μm to about 50 μm, about 20 μm to about 100 μm, about 20 μm to about 150 μm, about 20 μm to about 200 μm, about 20 μm to about 250 μm, about 20 μm to about 300 μm, about 25 μm to about 50 μm, about 25 μm to about 100 μm, about 25 μm to about 150 μm, about 25 μm to about 200 μm, about 25 μm to about 250 μm, about 25 μm to about 300 μm, about 50 μm to about 100 μm, about 50 μm to about 150 μm, about 50 μm to about 200 μm, about 50 μm to about 250 μm, about 50 μm to about 300 μm, about 100 μm to about 150 μm, about 100 μm to about 200 μm, about 100 μm to about 250 μm, about 100 μm to about 300 μm, about 150 μm to about 200 μm, about 150 μm to about 250 μm, about 150 μm to about 300 μm, about 200 μm to about 250 μm, about 200 μm to about 300 μm, or about 250 μm to about 300 μm. In some embodiment, the width of the conductive feedthrough may be about 10 μm, about 20 μm, about 25 μm, about 50 μm, about 100 μm, about 150 μm, about 200 μm, about 250 μm, or about 300 μm. In some embodiment, the width of the conductive feedthrough may be at least about 10 μm, about 20 μm, about 25 μm, about 50 μm, about 100 μm, about 150 μm, about 200 μm, about 250 μm, or more. In some embodiment, the width of the conductive feedthrough may be at most about 300 μm, about 250 μm, about 200 μm, about 150 μm, about 100 μm, about 50 μm, about 25 μm, about 20 μm, about 10 μm, or less.

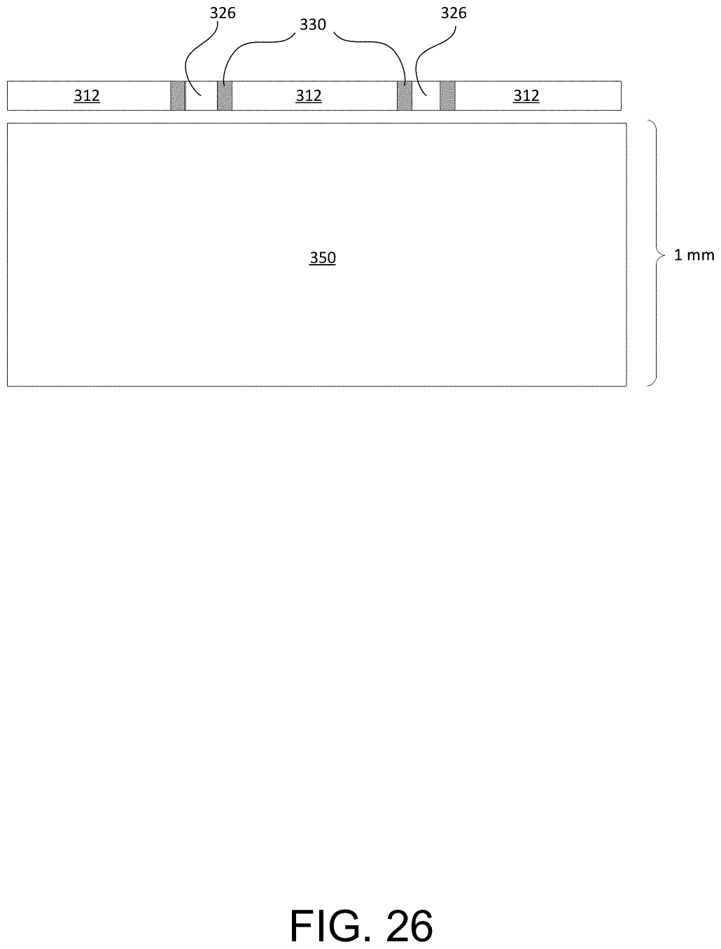

FIG. 26 shows the feedthrough device 340 in proximity with a conductive block 350, in accordance with some embodiments. The conductive block 350 may comprise a metal or metal alloy, for example platinum, iridium, niobium, chromium, scandium, titanium, vanadium, manganese, iron, cobalt, nickel, copper, zinc, yttrium, zirconium, gold, mercury, molybdenum, silver, tantalum, tungsten, aluminum, silicon, phosphorous, tin, an oxide of any of the preceding or any combination thereof. In other embodiments, the material may be a conductive ceramic such as TiN, conductive SiN, Indium tin oxide, etc.

In some embodiments, the conductive block 350 may comprise a same material as the patterned matching block 320. In other embodiments, the conductive block 350 and the patterned matching block 320 may comprise different materials. For example, in some embodiments, the conductive block 350 may comprise a platinum-iridium alloy, and the patterned matching block 320 may comprise niobium. In some embodiments, the conductive block 350 may comprise two or more materials. For example, the conductive block 350 may comprise a first portion made of tungsten, and a second portion made of platinum iridium. The first and second portions may be bonded or fused to each other, for example using compression bonding, friction welding, or diffusion bonding. The first portion (e.g. tungsten) may constitute a bulk of the conductive block, and may be used to provide stiffness/rigidity along the length of the microwires to be fabricated. The second portion (e.g. platinum iridium) may be used for the tips of the microwires, and may comprise a material that allows for enhanced neuronal recording. In some embodiments, the conductive block 150 may comprise a titanium-aluminum-vanadium alloy. It should be appreciated that two or more blocks of different materials (or alternating same materials) can be fused together to form the conductive block 350. The conductive block 350 may have a thickness ranging from about 100 um to about 2 mm. In some embodiments, the thickness of the conductive block 350 may be about 1 mm.

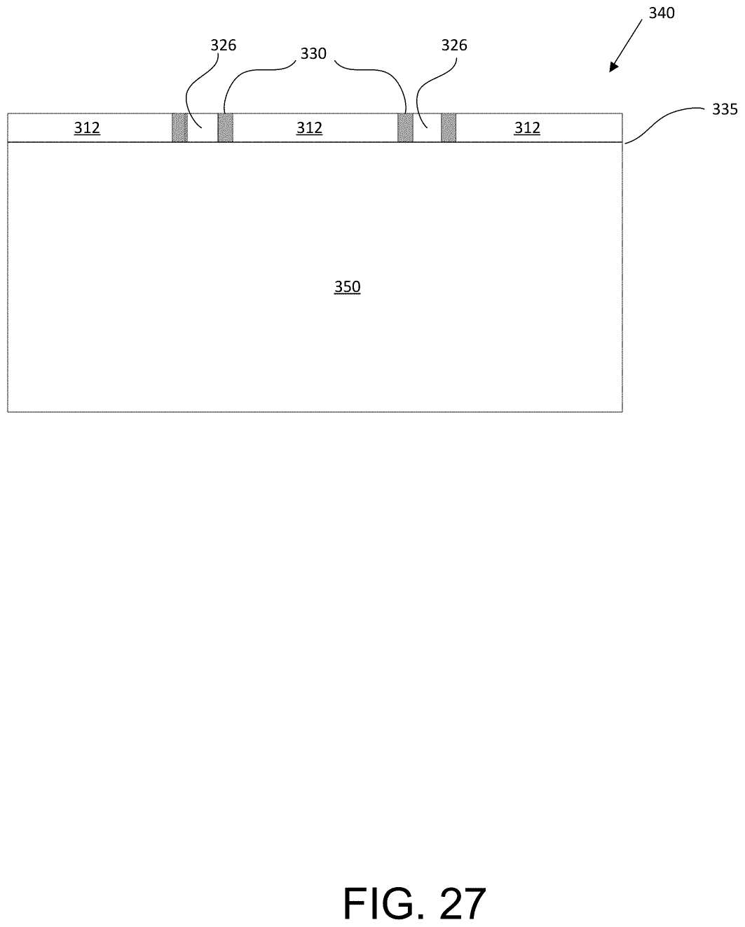

FIG. 27 shows the bonding of the feedthrough device 340 with the conductive block 350, in accordance with some embodiments. The feedthrough device 340 and the conductive block 350 may be brought into proximity with each other and bonded together. In some embodiments, the feedthrough device 340 and the conductive block 350 may be bonded to each other by applying pressure and reflowing a bonding material (e.g. gold) therebetween, for example using thermocompression bonding.

In some embodiments, the feedthrough device 340 and the conductive block 350 may be bonded to each other by diffusion bonding. A diffusion bond 335 may be formed at an interface between the mating surfaces of the feedthrough device 340 and the conductive block 350. Diffusion bonding may include pressure joining, auto-vacuum welding, thermocompression welding, solid-state or solid-phase welding. The diffusion bonding may be carried out in a vacuum chamber, which can protect the workpieces against intensive oxidation in high temperatures needed for the diffusion bonding process. Diffusion bonding can occur by creating surface deformation (e.g. crushing surface asperities, plastic deformation, etc.) and allowing interdiffusion of atoms across the interface of the joint or weld (e.g. solid-state diffusion). The transfer of atoms can take place through the lattice of crystalline solids at the joining interface, which may include exchange of places between adjacent atoms, motion of interstitial atoms, motion of vacancies in the crystalline lattice structures. In some of the embodiments disclosed herein, diffusion bonding can be used to join various types of metallic, ceramic, and crystalline materials (e.g. feedthrough device 340 and conductive block 350).

Process parameters (e.g. temperature, pressure, holding time, rate of heating, rate of cooling, and/or surface treatment) can be controlled or optimized to remove impurities from the joining surfaces, to enable diffusion bonding between similar or dissimilar materials to form a heterogenous bond (e.g. between two or more of the following: feedthrough device 340, conductive block 350, insulating portion 312, niobium, sapphire, platinum-iridium alloy, sapphire, gold, titanium, etc.). Process conditions including temperature, pressure, rate of heating, rate of cooling, and/or surface treatment can be optimized to produce plastic deformation locally at the joint surfaces that allows creep and diffusion to seal the interface and produce a bond. In some cases, the bonding temperature can be anywhere between a percentage or range of percentages (e.g. between 50% to 70%) of the melting point(s) of the material(s) being joined. The physical state of the surfaces to be joined can be important to the quality of the diffusion bonding. In some cases, the welding surface of the joining materials can be pretreated and optimized for diffusion bonding. The pretreatment may include processes such as machining, polishing, etching, chemical cleaning, coating, material creeping, etc. In some cases, when the two materials being bonded have different mechanical, physical, and chemical properties (e.g. embodiments of the feedthrough device 340 and the conductive block 350 described herein), a high quality bond can be achieved by using one or more intermediate layers comprising one or more other types of materials (e.g. a metallic alloy).

In some embodiments, a conductive block (e.g. platinum block) and a feedthrough dev as described elsewhere herein can be directly bonded together via diffusion bonding. The diffusion bonding can be conducted using a homogenous block of material. Alternatively, the diffusion bonding can be performed using a heterogeneous block comprising of two or more different materials. For example, the heterogeneous block may include a different material that forms either part of the block to create a multi-metal layered structure when cut. In some embodiments, a secondary material may be applied to the bulk of the block before bonding to enable a diffusion bond. For example, if a first material (e.g. silicon) is unable to be bonded directly to the feedthrough device, a second material (e.g. platinum) can be deposited on the silicon first, and the second material (e.g. platinum layer) may then be diffusion bonded to the hermetic feedthrough device. In some other embodiments, the bonding can be carried out using other techniques such as ultrasonic bonding/welding, friction welding, electric welding, or vacuum cementing.

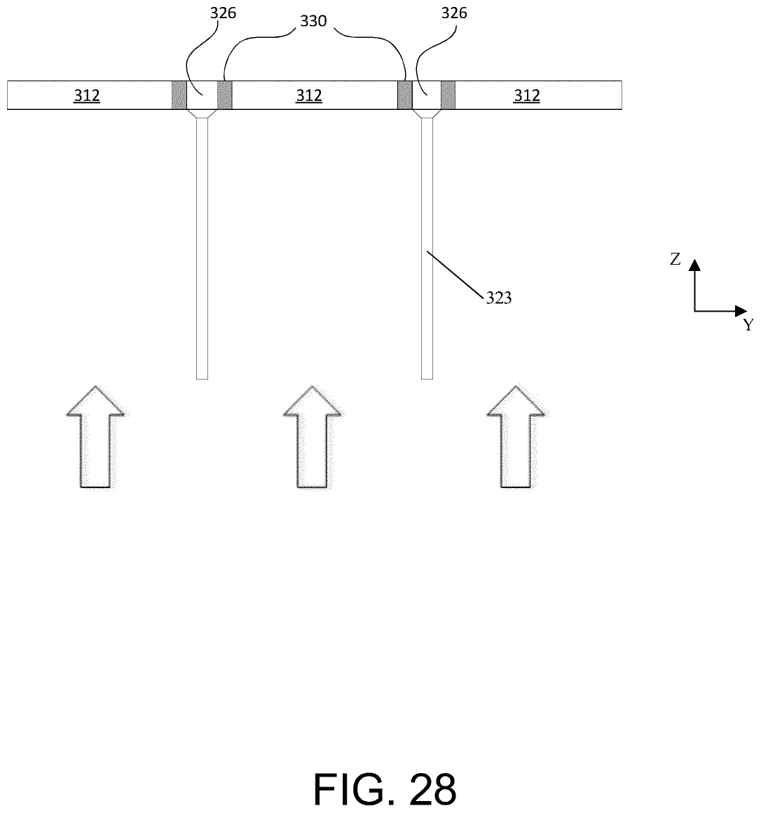

FIG. 28 shows material of the conductive block being removed to form high aspect ratio elongated or extended protrusions 323, in accordance with some embodiments. The high aspect ratio elongated or extended protrusions may include for example microwires, fins, plates and the like. The elongated protrusions 323, (e.g. a set of parallel slit/slots) can be formed by machining back a block illustrated by the arrows shown in FIG. 28. In some embodiments, the direction may be along the X-direction (FIG. 28, not shown). The machining process may include electric discharge machining (EDM), mechanical milling, LIGA, inductively coupled plasma (ICP) etching and the like. In some embodiments, in the block 350 a set of slits/slots having a relatively small width (e.g. 30 um) and deep (e.g. on the order of several hundred microns) can be cut, as shown in FIG. 28. In some embodiments, the block 350 can be milled back using wire-EDM, which is a metal-working process whereby material is removed by an electro-thermal erosion mechanism. Wire-EDM can be used to fabricate metallic microelectrodes with high aspect ratio since there is no cutting force involved in material removal. Wire-EDM generally occurs in a controlled environment and utilizes precision actuation stages to enable positioning with micrometer accuracy. Wire-EDM can be used to cut slits/slots having a relatively small width (e.g. 30 um) and deep (e.g. on the order of several hundred microns) in a metal block 350. In some embodiments, the elongated protrusions 323 can be made wider towards the base (e.g. as shown in FIG. 28) to increase hermeticity, or to make the elongated protrusions 323 mechanically more stable. The base may exhibit a high level of hermeticity, and be substantially impermeable to fluids (e.g. water vapor) and ions. In some embodiments, the base portion of the microwires may be chamfered, as shown in FIG. 28.

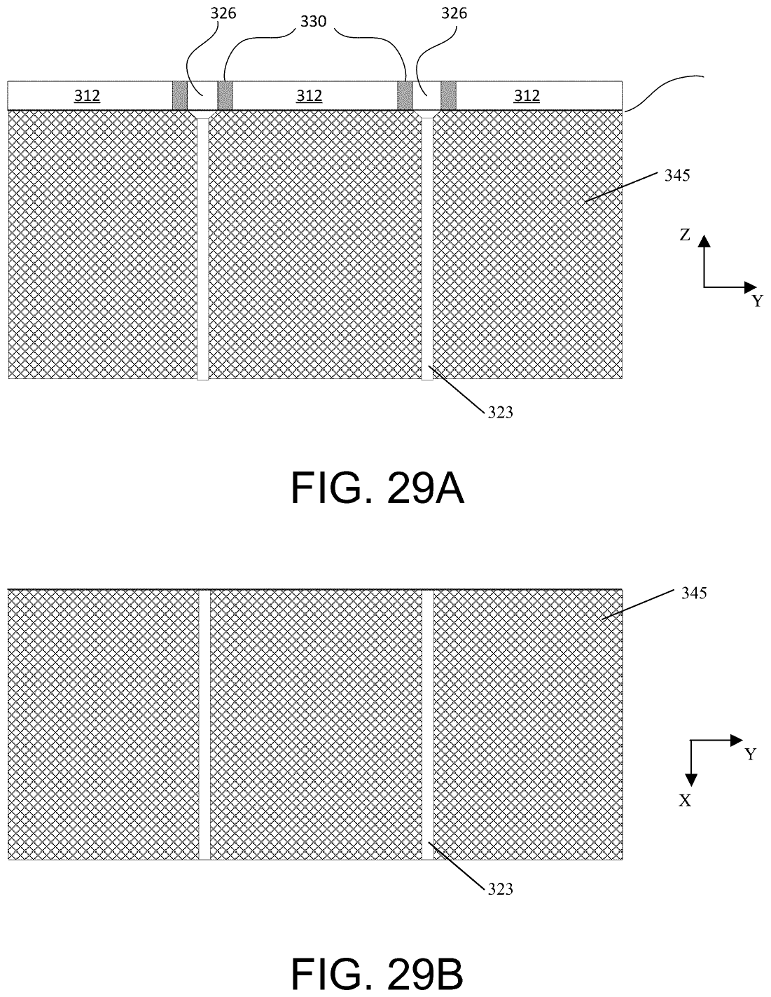

Next, a support material 345 may be deposited into the spaces between the elongated protrusions (e.g. 323, as shown in FIG. 29A (sectional view) and FIG. 29B (planar view). The support material 345 can provide support and structural rigidity to the extended protrusions 323 as they undergo machining in a second direction (e.g. Y-axis). This can reduce vibration and unwanted movements during the EDM process in the second direction, which may affect the geometric accuracy of the microstructures. In addition, the support material 345 can help to absorb or dissipate machining heat, thereby reducing the risk of the microwires deforming or breaking due to excessive absorption of Joule heat. The support material may also improve the quality of the machining surface finish.

The support material 345 may be any material that has a relatively low melting point, and can be reflowed or dispensed into the spaces between the extended protrusions 323 to form a monolithic solid block-shaped object. For example, the support material 345 may include gold, indium, tin, or a solder alloy. In some embodiments, the reflow of the support material 345 may be performed in a low pressure environment under vacuum.

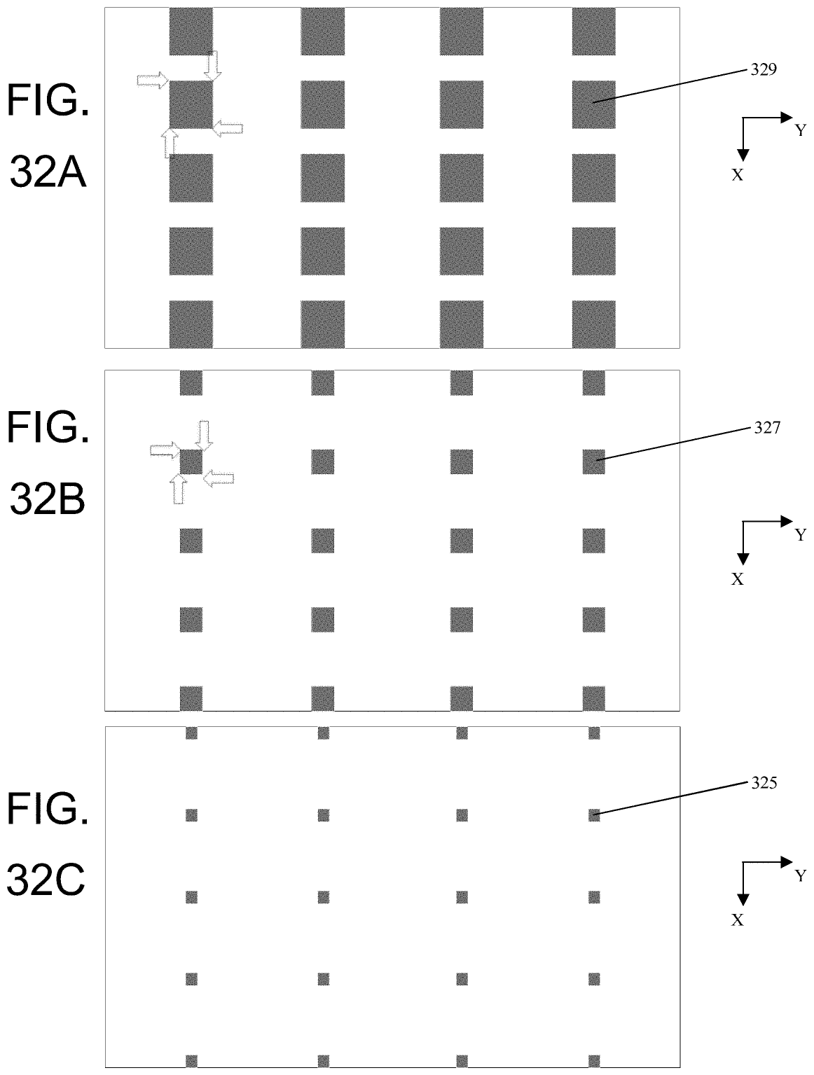

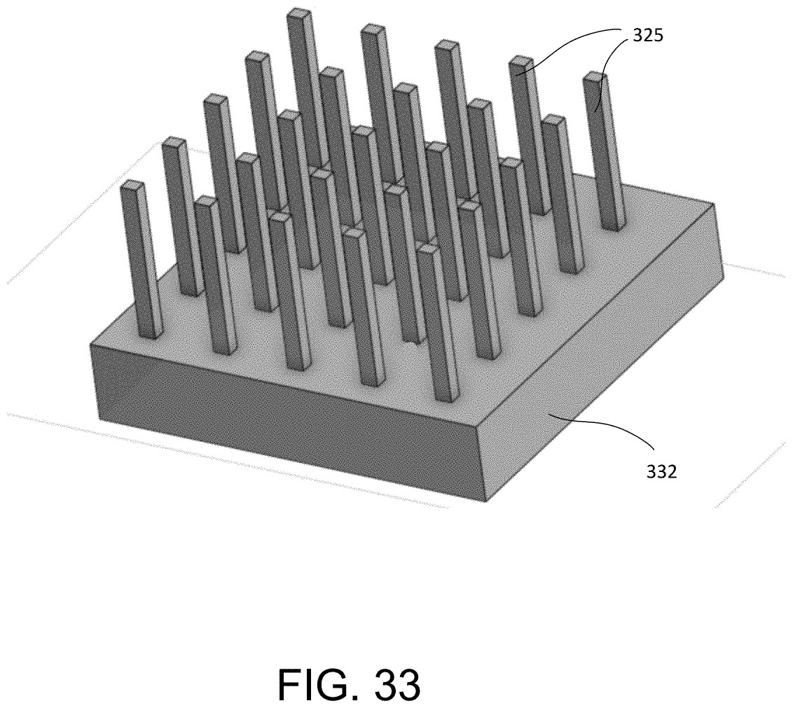

By cutting a set of parallel slits and then rotating the work piece (block) by an angle (e.g. 90 degrees) and repeating the cutting process, a microstructure array can be formed, for example shown in FIG. 32C and FIG. 33. Although FIG. 32C and FIG. 33 show an orthogonal microstructure array, the present disclosure is not limited thereto. For example, three sets of cuts at 60 degrees can be carried out to form a triangular patterned microstructure array.

The support material 345 may be made of any appropriate material that is biocompatible. In some embodiments, the biocompatible material may comprise gold, copper, platinum, silver, or any metallic alloy. In some embodiments, the support material 345 may comprise a semiconductor, a conductive polymer, or a conductive composite material. In some embodiments, the support material 345 may comprise an insulating material such as silicone compounds (e.g., polydimethylsiloxane (PDMS), poly(methyl methacrylate) (PMMA), etc.), medical-grade epoxies, organic polymer encapsulants, composite materials, and the like. In some embodiments, the support material 345 may be chosen such that the material is capable of dissolving within a subject's body, and thus disappears after the support material 345 has served its function of stabilizing the wires during the EDM process.

In some other embodiments, the support material 345 need not be biocompatible. In some embodiments, the support material 345 may be conductive. In some alternative embodiments, the support material 345 may be non-conductive. The support material 345 may also be deposited using low-stress methods such as electroplating.

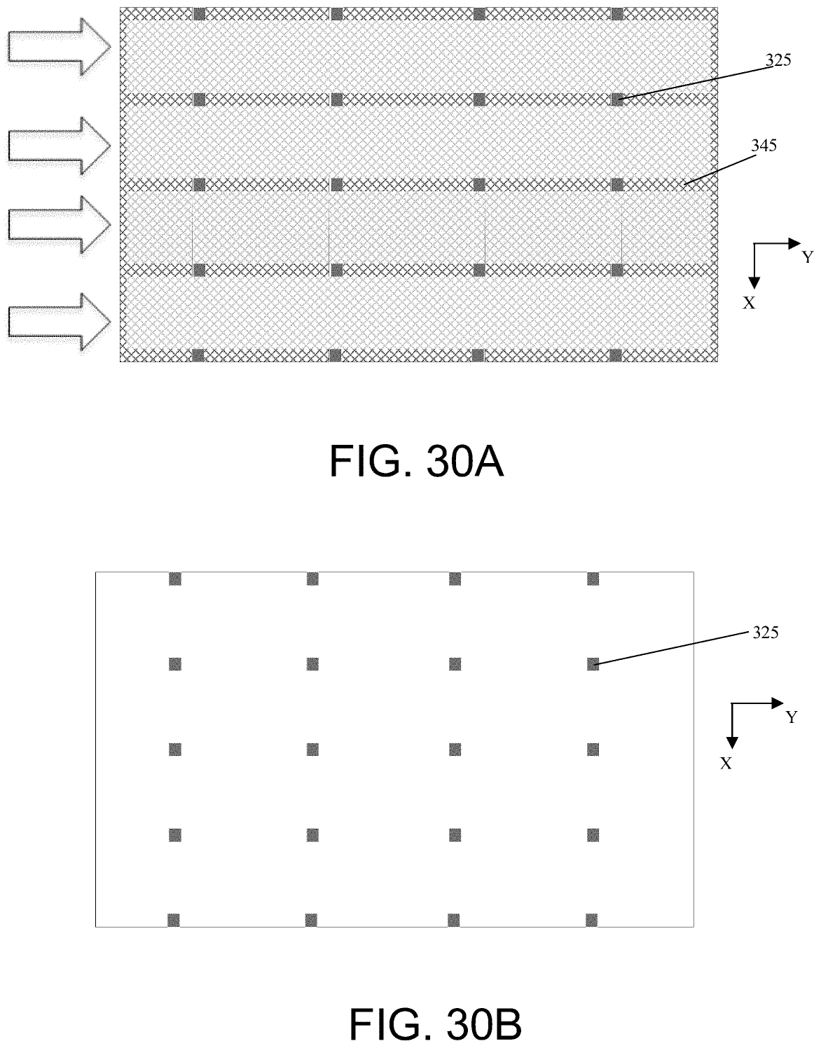

FIGS. 30A and 30B show microwires being formed and isolated from one another, in accordance with some embodiments. FIG. 30A shows the machining in the second direction (e.g. along the Y-axis) to form a plurality of stand-alone microwires 325. As described above, the support material 345 can provide support during the subsequent machining and prevent the microstructures from yielding or buckling during the EDM process.

After the microwires 325 have been formed, the support material 345 can be removed, resulting in the structure shown in FIG. 32C (planar view) and FIG. 33 (perspective view). The support material 345 may be etched using a preferential etching process that removes the support material 345 with little impact on the microwires 325 and the substrate on which the microwires are supported.

Referring to FIG. 13, 21A or 31B, in some embodiments, the insulating portions may have a thickness ranging from about 50 um to about 400 um. A base portion (e.g. flange) of the microwires may have a thickness ranging from about 10 μm to about 300 um. A distal portion of adjacent microwires may be spaced apart by a distance ranging from about 40 um to about 250 um. A base portion of adjacent microwires may be spaced apart by a distance ranging from about 10 um to about 150 um. The microwires may have a pitch spacing ranging from about 50 um to about 250 um. In some embodiments, a microwire may have a shaft that has a wider end closer to the base portion (e.g., closer to feedthrough substrate) and a thinner distal end (e.g., opposite to the base portion, or farther from the feedthrough substrate). The width or diameter of the shaft may decrease monotonically from the wider end to the thinner end as described herein. In some embodiments, the width or diameter of the shaft may decrease unevenly or in steps.

Each microwire may have a width (e.g., at the wider end) ranging from about 10 micrometers (μm) to about 60 μm. In some embodiment, each microwire may have a width of about 5 μm to about 10 μm, about 5 μm to about 15 μm, about 5 μm to about 20 μm, about 5 μm to about 25 μm, about 5 μm to about 30 μm, about 5 μm to about 35 μm, about 5 μm to about 40 μm, about 5 μm to about 45 μm, about 5 μm to about 50 μm, about 5 μm to about 55 μm, about 5 μm to about 60 μm, about 10 μm to about 15 μm, about 10 μm to about 20 μm, about 10 μm to about 25 μm, about 10 μm to about 30 μm, about 10 μm to about 35 μm, about 10 μm to about 40 μm, about 10 μm to about 45 μm, about 10 μm to about 50 μm, about 10 μm to about 55 μm, about 10 μm to about 60 μm, about 15 μm to about 20 μm, about 15 μm to about 25 μm, about 15 μm to about 30 μm, about 15 μm to about 35 μm, about 15 μm to about 40 μm, about 15 μm to about 45 μm, about 15 μm to about 50 μm, about 15 μm to about 55 μm, about 15 μm to about 60 μm, about 20 μm to about 25 μm, about 20 μm to about 30 μm, about 20 μm to about 35 μm, about 20 μm to about 40 μm, about 20 μm to about 45 μm, about 20 μm to about 50 μm, about 20 μm to about 55 μm, about 20 μm to about 60 μm, about 25 μm to about 30 μm, about 25 μm to about 35 μm, about 25 μm to about 40 μm, about 25 μm to about 45 μm, about 25 μm to about 50 μm, about 25 μm to about 55 μm, about 25 μm to about 60 μm, about 30 μm to about 35 μm, about 30 μm to about 40 μm, about 30 μm to about 45 μm, about 30 μm to about 50 μm, about 30 μm to about 55 μm, about 30 μm to about 60 μm, about 35 μm to about 40 μm, about 35 μm to about 45 μm, about 35 μm to about 50 μm, about 35 μm to about 55 μm, about 35 μm to about 60 μm, about 40 μm to about 45 μm, about 40 μm to about 50 μm, about 40 μm to about 55 μm, about 40 μm to about 60 μm, about 45 μm to about 50 μm, about 45 μm to about 55 μm, about 45 μm to about 60 μm, about 50 μm to about 55 μm, about 50 μm to about 60 μm, or about 55 μm to about 60 μm. In some embodiment, each microwire may have a width of about 5 μm, about 10 μm, about 15 μm, about 20 μm, about 25 μm, about 30 μm, about 35 μm, about 40 μm, about 45 μm, about 50 μm, about 55 μm, or about 60 μm. In some embodiment, each microwire may have a width of at least about 5 μm, about 10 μm, about 15 μm, about 20 μm, about 25 μm, about 30 μm, about 35 μm, about 40 μm, about 45 μm, about 50 μm, about 55 μm, or more. In some embodiment, each microwire may have a width of at most about 60 μm, about 55 μm, about 50 μm, about 45 μm, about 40 μm, about 35 μm, about 30 μm, about 25 μm, about 20 μm, about 15 μm, about 10 μm, or less.

In some embodiments, a flange may be connected to a feedthrough hole at the proximal end of the microwire (e.g., base of the microwire closer to the substrate). The flange may be used to connect a microwire to the feedthrough. The flange may support the microwire structurally at the end closer to the feedthrough. In some cases, the flange may provide a joint section that is larger (e.g., thicker, larger diameter or both) than a microwire forming a stronger mechanical joint. The flange may also facilitate forming an array with substantially parallel microwires. In some cases, the flange may be used for fitting, where the diameter of a microwire may be different from an inner diameter of a feedthrough. In some cases, where a material of a microwire may be different from a material of a feedthrough, a flange may separate and shield the different materials. For example, a flange comprising biocompatible material may be used to prevent a contact (e.g., human skin, hair, etc.) with a feedthrough material that may not be biocompatible. In some cases, the flange may reduce manufacturing burden or cost. For example, a flange may be used to circumvent a need for a minimum requirement for an internal corner radius in a manufacturing process. The flange may be a ring, or a donut shape structure with an inner diameter or width and an outer diameter or width. The base of a microwire may go through the inner portion (e.g., a hole) of the flange to contact the feedthrough. A flange may have a size (e.g., an outer diameter or an inner diameter) that may be at least half a width or diameter of the conductive feedthrough (e.g., conductive pad 326) or more. In some cases, the flange may have a width or a diameter of between about 50% to about 150% of the conductive feedthrough. In some embodiments, a ratio of the flange size to a diameter of the feedthrough on which the microwire is located may be less than 50%. In some embodiments, a ratio of the flange size to a diameter of the feedthrough on which the microwire is located may be about 50%, about 55%, about 60%, about 70%, about 80%, about 90%, about 100%, or a percentage between any two percentage amounts mentioned hereinbefore.

In some cases, a flange may be used prior, during, or after soldering or brazing. In some cases, the flange may be used in addition to diffusion bonding or welding. A flange may be soldered, brazed, diffusion bonded, or welded to a feedthrough substrate, a microwire, or both.

In some embodiments, a microwire may be connected to the feedthrough using solder or braze. The microwire end closer to the feedthrough may be connected to a feedthrough in a substrate (e.g., ceramic substrate) using solder or braze without causing electric shorting between electrodes (e.g., microwires). In some embodiments, a filler material used for solder or braze may be a biocompatible material (e.g., gold, platinum or alloys thereof). A biocompatible material for solder may comprise gold, nickel, titanium, niobium, palladium, tungsten, silver, a combination thereof, or an alloy material thereof. Alternatively, a solder or braze may diffuse (e.g., interdiffuse) with a microwire or a feedthrough material during joining. In some cases, a solder or braze may form an alloy with the microwire, the feedthrough or both. The solder or braze region may have a thickness of about no more than 200 micrometers (μm). In some embodiments, the thickness of a solder or braze in between a microwire and a feedthrough may be from about 2 μm to about 200 μm, about 2 μm to about 50 μm, about 50 μm to about 150 μm, about 100 μm to about 150 μm, about 120 μm to about 180 μm, or about 140 to about 160 μm. In some cases, the thickness of a solder or braze in between a microwire and a feedthrough may be from about 2 μm to about 50 μm.

In some embodiments, brazing may be applied at a temperature of at least about 450° C. A biocompatible filler material used in brazing may be a metal or metal alloy (e.g., titanium, zirconium, manganese, or alloys thereof). In some embodiments, soldering may be applied at a temperature of at most about 450° C. A biocompatible filler material used in soldering may be a metal or metal alloy (e.g., zinc, silver, gold, or alloys thereof). A biocompatible material for braze may comprise gold, nickel, titanium, niobium, palladium, tungsten, silver, a combination thereof, or an alloy material thereof.

In the examples shown in FIGS. 17, 29A, and 29B, although the cuts may be made in two orthogonal directions (X and Y axes), the present disclosure is not limited thereto. For example, the cuts may be made in three or more different directions, at a variety of different angles (e.g. 45 degrees, 60 degrees, etc.) The cuts may be made in n number of different directions, wherein n may be an integer equal or greater than 3 to form a polygonal shaped elongated protrusion (e.g. microwire, rod, pin, etc.) In some cases, n may be equal or greater than 100, 200, 300, 500, 1000, or more to form an almost circular/cylindrical protrusion (e.g. microwire, rod, pin, etc.). In some embodiments, the sides of the polygon shape elongated protrusion may have different aspect ratios (e.g. width or surface area).

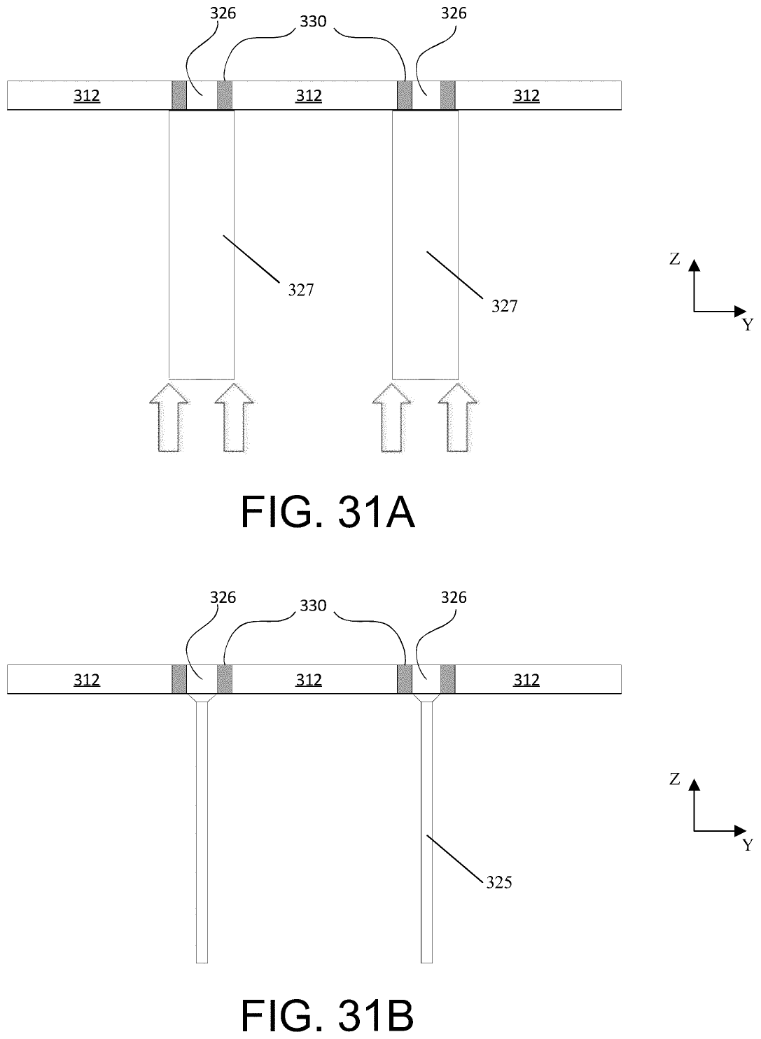

FIGS. 31A and 31B illustrate examples of other processes for fabricating a microwire array (cross section view), in accordance with some other embodiments. As shown in FIGS. 31A and 31B, an initial structure can be sequentially thinned to form thinner microwires. For example, a first cut may be made (e.g. using EDM) to form microwires 327 having a first dimension (e.g. width, diameter, or thickness). Next, the microwires 327 may be cut further (using EDM, or any other milling or etching process) to thin the microwires 327 to form microwires 325 having a second dimension (e.g. width, diameter, or thickness). The second dimension may be less than the first dimension. For example, the second dimension may be at least 5%, 10%, 20%, 30%, 40% or 50% smaller than the first dimension. As shown in FIG. 31B, the microwires 325 can be formed wider towards the base. The widened base can increase hermeticity, and/or cause the microwires 325 to be mechanically more stable. The base may exhibit a high level of hermeticity, and be substantially impermeable to fluids (e.g. water vapor) and ions. In some embodiments, the base portion of the microwires may be chamfered, as shown in FIG. 31B. In some embodiments, a method of thinning the wires may be via ion bombardment, or ion milling. The thinning process may not only reduce the dimensions of the microwire, but can also remove any surface asperities or heat affected zones, smooth the surface of the wire. The process may also round the edge of the wire and create tapering along the wire, changing the wire cross section from square after EDM to round after milling or etching. The milling or etching process may also create a taper at the top of the electrode, forming a tip. The process may also create a taper down the length of the wire, dependent on the process conditions (e.g. ion energy, chemistry, or directional angle of the process).

FIGS. 32A-32C illustrate planar views as an array of microwires is being reduced in dimension. Multiple cuts may be made in two or more different directions or axes. For example, the array of microwires may be initially cut to the size shown in FIG. 32A using any of the processes described herein (e.g. EDM). Next, the microwires may be cut in a plurality of directions and along a plurality of faces of the microwires, to reduce the dimension of the microwires to yield thinner microwires for example as shown in FIG. 32B. Similarly, the microwires of FIG. 32B may be further cut in a plurality of directions and along a plurality of faces of the microwires, to reduce the dimension of the microwires to yield even thinner microwires for example as shown in FIG. 32C. Any number of cuts in any direction/axis may be contemplated, to reduce one or more dimensions (e.g. thickness, width, diameter, or length) of one or more microwires within the array. In some embodiments, a method of thinning the wires may be via ion bombardment, or ion milling. The thinning process may not only reduce the dimensions of the microwire, but can also remove any surface asperities or heat affected zones, smooth the surface of the wire. The process may also round the edge of the wire and create tapering along the wire, changing the wire cross section from square after EDM to round after milling or etching. The milling or etching process may also create a taper at the top of the electrode, forming a tip. The process may also create a taper down the length of the wire, dependent on the process conditions (e.g. ion energy, chemistry, or directional angle of the process).

In some embodiments, sharpened tips (e.g., conical tips) may be formed at the distal ends of the microwires. The sharpened tips may be formed by etching the conductive block along a set of predefined planes, by ion beam material removal, by laser milling, by electric discharge machining, or by mechanical milling. The sharpened tips may have a beveled cut surface. In some embodiments, the beveled cut surface may have an angle of about 5 degrees, 10 degrees, 20 degrees, 30 degrees, 40 degrees, 50 degrees, 60 degrees, 70 degrees, 80 degrees, 90 degrees, or any angle within a range between any two of the preceding values. The sharpened tips may be conical. The sharpened tips can help to facilitate insertion into brain tissue.

In some embodiments, the sharpened tip (e.g., conical tip) may have a widest radius of between about 1 micrometer (μm) to about 10 μm. In some embodiment, the sharpened tip (e.g., conical tip) may have a widest radius of about 1 μm to about 2 μm, about 1 μm to about 3 μm, about 1 μm to about 4 μm, about 1 μm to about 5 μm, about 1 μm to about 6 μm, about 1 μm to about 7 μm, about 1 μm to about 8 μm, about 1 μm to about 9 μm, about 1 μm to about 10 μm, about 2 μm to about 3 μm, about 2 μm to about 4 μm, about 2 μm to about 5 μm, about 2 μm to about 6 μm, about 2 μm to about 7 μm, about 2 μm to about 8 μm, about 2 μm to about 9 μm, about 2 μm to about 10 μm, about 3 μm to about 4 μm, about 3 μm to about 5 μm, about 3 μm to about 6 μm, about 3 μm to about 7 μm, about 3 μm to about 8 μm, about 3 μm to about 9 μm, about 3 μm to about 10 μm, about 4 μm to about 5 μm, about 4 μm to about 6 μm, about 4 μm to about 7 μm, about 4 μm to about 8 μm, about 4 μm to about 9 μm, about 4 μm to about 10 μm, about 5 μm to about 6 μm, about 5 μm to about 7 μm, about 5 μm to about 8 μm, about 5 μm to about 9 μm, about 5 μm to about 10 μm, about 6 μm to about 7 μm, about 6 μm to about 8 μm, about 6 μm to about 9 μm, about 6 μm to about 10 μm, about 7 μm to about 8 μm, about 7 μm to about 9 μm, about 7 μm to about 10 μm, about 8 μm to about 9 μm, about 8 μm to about 10 μm, or about 9 μm to about 10 μm. In some embodiment, the sharpened tip (e.g., conical tip) may have a widest radius of about 1 μm, about 2 μm, about 3 μm, about 4 μm, about 5 μm, about 6 μm, about 7 μm, about 8 μm, about 9 μm, or about 10 μm. In some embodiment, the sharpened tip (e.g., conical tip) may have a widest radius of at least about 1 μm, about 2 μm, about 3 μm, about 4 μm, about 5 μm, about 6 μm, about 7 μm, about 8 μm, about 9 μm, or more. In some embodiment, the sharpened tip (e.g., conical tip) may have a widest radius of at most about 10 μm, about 9 μm, about 8 μm, about 7 μm, about 6 μm, about 5 μm, about 4 μm, about 3 μm, about 2 μm, about 1 μm, or less.

In some embodiments, an electrochemical modification process can be used to form sharpened tips. Electrochemical modification can be carried out in an electrically conductive bath. In the case of subtractive electrochemical modification, the bath may contain reactive elements such as sodium or potassium hydroxide, at a concentration above 0.01M and below 10M. The bath may also contain acids such as sulfuric or phosphoric acid. The subtractive process may be performed under an applied voltage at a metal core, which may typically be positive. The specific formulation used in the bath can vary depending on the selected material, as known to those skilled in the art.

In some embodiments, after the microwires have been cut by EDM, the microwires may be slightly etched further to make the microwires round and smooth along the length of the microwires, and to reduce the diameter of the microwires. In some cases, further electrosharpening of the tips of the microwires may be performed. The microwires may also be smoothed by using a second die-sink EDM process, where a preformed block with holes is aligned to the grid and used to do the final forming step of the microwires.

In some embodiments (not shown), one or more of the microwires 325 may be formed having a non-straight structure with one or more curved sections. In some cases, each of the microwires 325 may be formed having a non-straight structure with multiple curved sections along its length.

Subtractive modification may result in two types of general geometries: (1) smooth and flat material removal (often called electropolishing), or (2) the sharpening of the wire to an apex (called electrosharpening). The geometries may depend on factors such as mass transport of chemical reagents to the material surface, applied potential at the electrode surface, solution composition, temperature, applied forces to the electrode during the process, and the like.

The additive process may be carried out using a solution that contains the dissolved ions of the material. An electric potential may be applied to the electrode which is undergoing modification, but this may not be necessary. When a potential is applied, it may typically be negative, which causes the dissolved ions to reduce onto the surface of the material as a solid. When a potential is not applied to the electrode undergoing modification, a reducing agent may typically be included in the solution to enable the application of the material coating. Growth of material at the surface of the electrode may take on different forms and morphologies depending on the deposition conditions, including reagent chemistry, chemical mass transport, temperature, electric potential applied to the electrode, and the like. In some embodiments, deposition of the additive solution may be carried out by local deposition using a pipette or micropipette or any other suitable tool that is capable of dispensing volumes from 1 um3 to 10000 um3. The additive solution may also be applied via the use of surface tension, for example by dipping the array into a solution and withdrawing it so that the surface tension of the liquid holds it in place. The additive solution may also be added via condensation, being nucleated at the tip of the wire via an evaporative or Ostwald ripening process. The process may also be carried out by the formation of a mask layer on top of the electrode, after which material can be deposited by a sputtering, evaporation, or electrochemical process.

In some embodiments, the sharpened tips of the microwires may be electrochemically coated with a low-impedance coating, such as iridium oxide (or other transition-metal oxide, such as MnO₂, etc.), a conductive polymer (e.g., PEDOT, etc.), or a material promoting a high surface area (e.g. carbon nanotubes, platinum black, nanoparticle composites, and the like). The surface modification can decrease the interfacial electrical impedance between the exposed conductor core and brain tissue, thereby increasing the sensitivity of the neural-activity recording.

In some embodiments, designated weak zones may be formed along different sections of the microwires during the wire-EDM process. These designated weak zones can allow the microwires to fold or crumple in a controlled fashion when the insertion force of the microwires into brain tissue exceeds a predefined threshold.

In some embodiments, the microwires may be coated with an insulating layer (not shown) along the length of the microwires. In some embodiments, the insulating layer may cover the microwires, a side of the feedthrough device proximal to the microwires, or both. The insulating layer may be made of any appropriate material that is biocompatible and suitable for placement or insertion into neural matter. The insulating layer may be formed by a thermal drawing process, for example by drawing glass as a cladding over the microwires. The insulating layer may comprise glass, or any other suitable insulating materials such as silicone compounds (e.g., polydimethylsiloxane (PDMS), poly(methyl methacrylate) (PMMA), etc.), high temperature deposited oxides, medical-grade epoxies, organic polymer encapsulants, composite materials, and the like. In some embodiments, the insulating layer may comprise a plurality of insulating layers having one or more different material properties (e.g., dielectric constant, chemical reactivity/resistance, hardness, etc.) The insulating thin film can provide electrical insulation. The insulating layer may improve electrical properties (e.g., impedance) of the device.

In some embodiments, the insulating layer may be a ceramic thin film. The thickness of the insulating layer (e.g., ceramic thin film) may be from about 0.2 micrometers (μm) to about 4 μm. In some cases, a tip of a microwire (e.g., an end farther from the feedthrough device) may be de-insulated by removing the insulating layer. The tip may be de-insulated using a subtractive technique (e.g., by laser or ion mill). Coating a microwire with an insulating thin film (e.g., ceramic thin film) and de-insulating the tip of the microwire can lead to modified (e.g., improved) electrical properties of the microwire (e.g., impedance). In some embodiments, an impedance of a microwire coated with a thin film of an insulating material (e.g., ceramic) with a de-insulated tip can have an impedance of about 50 kilo-ohms to about 5,000 kilo-ohms when tested at a frequency of 1 KHz, in biological saline solution. In some instances, the impedance may be less than about 50 kilo-ohms. In other cases, the impedance may be greater than about 5,000 kilo-ohms.

In some embodiment, the thickness of the insulating layer (e.g., ceramic thin film) may be about 0.2 μm to about 0.5 μm, about 0.2 μm to about 0.8 μm, about 0.2 μm to about 1.2 μm, about 0.2 μm to about 1.5 μm, about 0.2 μm to about 1.8 μm, about 0.2 μm to about 2 μm, about 0.2 μm to about 2.5 μm, about 0.2 μm to about 3 μm, about 0.2 μm to about 3.5 μm, about 0.2 μm to about 4 μm, about 0.5 μm to about 0.8 μm, about 0.5 μm to about 1.2 μm, about 0.5 μm to about 1.5 μm, about 0.5 μm to about 1.8 μm, about 0.5 μm to about 2 μm, about 0.5 μm to about 2.5 μm, about 0.5 μm to about 3 μm, about 0.5 μm to about 3.5 μm, about 0.5 μm to about 4 μm, about 0.8 μm to about 1.2 μm, about 0.8 μm to about 1.5 μm, about 0.8 μm to about 1.8 μm, about 0.8 μm to about 2 μm, about 0.8 μm to about 2.5 μm, about 0.8 μm to about 3 μm, about 0.8 μm to about 3.5 μm, about 0.8 μm to about 4 μm, about 1.2 μm to about 1.5 μm, about 1.2 μm to about 1.8 μm, about 1.2 μm to about 2 μm, about 1.2 μm to about 2.5 μm, about 1.2 μm to about 3 μm, about 1.2 μm to about 3.5 μm, about 1.2 μm to about 4 μm, about 1.5 μm to about 1.8 μm, about 1.5 μm to about 2 μm, about 1.5 μm to about 2.5 μm, about 1.5 μm to about 3 μm, about 1.5 μm to about 3.5 μm, about 1.5 μm to about 4 μm, about 1.8 μm to about 2 μm, about 1.8 μm to about 2.5 μm, about 1.8 μm to about 3 μm, about 1.8 μm to about 3.5 μm, about 1.8 μm to about 4 μm, about 2 μm to about 2.5 μm, about 2 μm to about 3 μm, about 2 μm to about 3.5 μm, about 2 μm to about 4 μm, about 2.5 μm to about 3 μm, about 2.5 μm to about 3.5 μm, about 2.5 μm to about 4 μm, about 3 μm to about 3.5 μm, about 3 μm to about 4 μm, or about 3.5 μm to about 4 μm. In some embodiment, the thickness of the insulating layer (e.g., ceramic thin film) may be about 0.2 μm, about 0.5 μm, about 0.8 μm, about 1.2 μm, about 1.5 μm, about 1.8 μm, about 2 μm, about 2.5 μm, about 3 μm, about 3.5 μm, or about 4 μm. In some embodiment, the thickness of the insulating layer (e.g., ceramic thin film) may be at least about 0.2 μm, about 0.5 μm, about 0.8 μm, about 1.2 μm, about 1.5 μm, about 1.8 μm, about 2 μm, about 2.5 μm, about 3 μm, about 3.5 μm, or more. In some embodiment, the thickness of the insulating layer (e.g., ceramic thin film) may be at most about 4 μm, about 3 μm, about 2.5 μm, about 2 μm, about 1.5 μm, about 1 μm, about 0.5 μm, or less.

A feedthrough device may be configured to have a leak rate of at most 10⁻⁴ atm·cc/s, where 1 atm·cc/s is equivalent to 60 standard cubic centimeters per minute (sccm). In some embodiments, the leak rate of the feedthrough device may be between about 10⁻¹⁰ atm·cc/s to about 10⁻⁴ atm·cc/s. In some cases, the leak rate of the feedthrough device may be less than about 10⁻¹⁰ atm·cc/s. The feedthrough device may be manufactured as a stand-alone entity. The microarray (or microwires) may be connected to the stand-alone feedthough device using any of the methods described herein. For example, a conductive block may be connected to the feedthrough device and microwires (or microelectrodes) may be formed by subtracting portions of the conductive block (e.g., a protoarray block). The microarray (or microwires) may be connected to the stand-alone feedthrough device without affecting or changing the hermiticity of the feedthrough device. In some embodiments, the assembly comprising the feedthrough device and the microwires may be coated with a thin insulating film (e.g., ceramic film) to achieve a hermeticity of about 10⁻¹⁰ atm·cc/s to about 10⁻⁴ atm·cc/s. The thin film coat may be removed on or around the tip (e.g., the conical tip, sharpened tip), which is the end farther away from the feedthrough, of the coated microwires to allow electrical current to pass. The tip may be de-insulated by a subtractive technique (e.g., laser or ion mill).

In some embodiments, the leak rate of the feedthrough device may be about 10⁻¹⁰ atm·cc/s to about 10⁻⁴ atm·cc/s, about 10⁻⁹ atm·cc/s to about 10⁻⁴ atm·cc/s, about 10⁻⁷ atm·cc/s to about 10⁻⁴ atm·cc/s, about 10⁻⁸ atm·cc/s to about 10⁻⁴ atm·cc/s, about 10⁻⁶ atm·cc/s to about 10⁻⁴ atm·cc/s, about 10⁻⁵ atm·cc/s to about 10⁻⁴ atm·cc/s, about 10⁻⁹ atm·cc/s to about 10⁻⁵ atm·cc/s, about 10⁻⁸ atm·cc/s to about 10⁻⁵ atm·cc/s, about 10⁻⁷ atm·cc/s to about 10⁻⁵ atm·cc/s, about 10⁻⁶ atm·cc/s to about 10⁻⁵ atm·cc/s, about 10⁻¹⁰ atm·cc/s to about 10⁻⁶ atm·cc/s, about 10⁻⁹ atm·cc/s to about 10⁻⁶ atm·cc/s, about 10⁻⁸ atm·cc/s to about 10⁻⁶ atm·cc/s, about 10⁻⁷ atm·cc/s to about 10⁻⁶ atm·cc/s, about 10⁻¹⁰ atm·cc/s to about 10⁻⁷ atm·cc/s, about 10⁻¹⁰ atm·cc/s to about 10⁻⁸ atm·cc/s, about 10⁻¹⁰ atm·cc/s to about 10⁻⁹ atm·cc/s, In some embodiments, the leak rate of the feedthrough device may be at least about 10⁻¹⁰ atm·cc/s, about 10⁻⁹ atm·cc/s, about 10⁻⁸ atm·cc/s, about 10⁻⁷ atm·cc/s, about 10⁻⁶ atm·cc/s, about 10⁻⁵ atm·cc/s, or about 10⁻⁴ atm·cc/s, or greater. In some embodiments, the leak rate of the feedthrough device may be at most about 10⁻⁴ atm·cc/s, about 10⁻⁵ atm·cc/s, about 10⁻⁶ atm·cc/s, about 10⁻⁷ atm·cc/s, about 10⁻⁸ atm·cc/s, about 10⁻⁹ atm·cc/s, about 10⁻¹⁰ atm·cc/s, or smaller.

Any of the devices herein may be bonded to a chip to form an active microprobe array. The chip may be an active device that is capable of recording voltage and/or generating current.

In some embodiments, the chip may be a display driver chip. The chip may be a high performance readout integrated circuit (ROIC) chip that has been configured for adapted for neural recording. The chip may comprise a plurality of pixels/electrodes. In some embodiments, the chip may be a m×n pixel read out integrated circuit (ROIC) imaging chip with a total of m×n pixels/electrodes over an array area. The array area may be given by X1×Y1. In some embodiments, X1=Y1 such that the array has a square shape. In other embodiments, X1≠Y1 such that the array has a rectangular shape. The chip can be configured to acquire data at a rate of millions of pixels per second. The chip may have an adjustable gain current amplifier in each pixel circuit can be controlled by a series of input and output boards through a computer. The chip may be a multiplexed current readout chip with a gain amplifier in each unit cell or pixel.

The chip may include an m×n two-dimensional array of bond pads corresponding to the pixel array. Each of the bond pads may be individually addressable and configured to drive a pixel on a separate display (e.g., an LED or LCD-based display, not shown). The bond pads may be spaced apart by a pitch px along an x-axis and by a pitch py along a y-axis. The pitches px and py may be constant or variable. The pitches px and py may be the same or different. In some embodiments, each of the pitches px and py may be at least 10 μm, 50 μm, 100 μm, 200 μm, less than 10 μm, or greater than 200 μm. The pitch of the bond pads on the chip may be customized based on the pitch of the conductive pads on the device.

The device herein is configured to be attached (mechanically and electrically connected) to a chip via conductive pads. The mechanical/electrical coupling may be provided by a plurality of interconnects (not shown) formed at an interface between the bond pads of the chip, and the conductive pads of the device. The microwires can be electrically connected to the plurality of bond pads of the chip via the plurality of interconnects formed between the chip and the device. The interconnects allow the electrodes at the distal portion of the microwires to be in electrical communication with the integrated circuit elements on the chip, during monitoring and/or stimulation of brain activity.

Neural-interface microprobe arrays of different lengths and other dimensions (width, etc.) may be used for different regions of the brain. The microprobe array described herein can be used to monitor and/or stimulate neural activity. In some embodiments, the microprobe probe may be inserted into a brain, such that the flexible distal portion of the microwires interfaces and is in contact with a target region of the neural matter. Neural activity in the target region can be monitored and/or stimulated via a plurality of electrical signals transmitted between the chip and the neural matter. The electrical signals may be transmitted through the plurality of microwires. In some embodiments, the electrical signals may be transmitted from the microprobe array to an external monitoring device via one or more wireless or wired communication channels.

In some embodiments, the implanted neural-interface microprobe array may be connected to the external world via a percutaneous wire. The percutaneous wire may be inserted through a patient's scalp. In other embodiments, the implanted neural-interface microprobe array may be connected to the external world via a wireless telemetry unit.

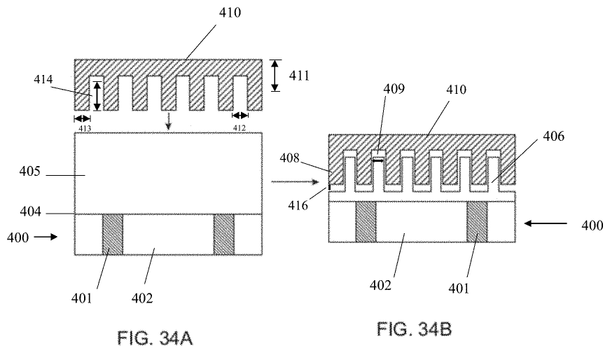

Further additional methods to form high aspect ratio protrusions such as microwires on a feedthrough plate are next described. FIGS. 34A and 34B show an example of a subtractive process, where material from a conductive block 405 can be removed to form high aspect ratio protrusions 406 on a feedthrough plate 400. The feedthrough plate 400 may be similar to the feedthrough device 340 or any of the feedthrough device/plate described elsewhere herein. The conductive block 400 and the elongated protrusions 406 may be similar to the conductive block 350 and the microwires 325 described herein. The feedthrough plate 400 may comprise a plurality of conductive pads 401 spaced apart and separated from one another by insulating portions 402, similar to the conductive pads 326 and the insulating portions 312 described herein. In some embodiments, the insulating portions 402 may comprise sapphire. In some embodiments, the conductive pads 401 may comprise niobium. The conductive block 405 may comprise a metal or metallic alloy, for example platinum, iridium, niobium, chromium, scandium, titanium, vanadium, manganese, iron, cobalt, nickel, copper, zinc, yttrium, zirconium, gold, mercury, molybdenum, silver, tantalum, tungsten, aluminum, silicon, phosphorous, tin, an oxide of any of the preceding or any combination thereof. In other embodiments, block 405 may be a conductive ceramic such as TiN, conductive SiN, Indium tin oxide, etc. In some embodiments, the conductive block 405 can be fused or bonded to the feedthrough plate 400 using any of the bonding methods described herein, for example using compression bonding, friction welding, or diffusion bonding. In some embodiments, the block 405 can be fused or bonded to one side of the feedthrough plate 404. In some embodiments, the block 405 may have a thickness ranging from 1 mm or greater.

FIGS. 34A and 34B show a plurality of elements that may be used in a subtractive process using a die, mold or template. In some embodiments, the subtractive process can include die-sink EDM or ram EDM, whereby a die is formed first and used to machine the block. In some embodiments, a first electrode may comprise a template tool 410 (e.g. a die-sink) and a second electrode may comprise the workpiece (e.g. the conductive block 405 to be machined). The template tool 410 can be made of a metal such as brass, copper, bronze, titanium, aluminum or a metallic alloy such as stainless steel, sintered copper/tungsten, or conductive material such as graphite. The template tool 410 can mirror the structure that is being formed out of the workpiece 405. The template tool 410 may comprise a base, a plurality of protrusions 408 extending from the base, and a plurality of indentations or cavities spaced between the protrusions 408. In some embodiments, the plurality of indentations or cavities can be used to form microwires in the workpiece (conductive block 405). The cavities of the template tool can be formed by deep hole drilling. In some embodiments, the cavities of the template tool can be formed using a high aspect ratio etching process (e.g. deep reactive ion etching). The size (e.g. width or diameter) of the cavities 412 may range from between about 10 um to about 100 um. The depth or length of the cavities 414 may range from 1 mm or more. The base of the template tool 415 may be about 10 um to about 100 um thick. The protrusions on the template tool 413 may be about 50 um to about 250 um wide. In some embodiments, the microwires 406 formed on the feedthrough plate 400 may have a wider base portion (not shown). The microwires 406 may be separated and isolated from one another electrically (not shown). In some embodiments, the microwires may have a width 409 of about 10 micrometers (μm) to about 60 μm.

The die-sink can be used to form one or more portions, or the entirety of the microwires. In some embodiments, the die-sink can be combined with wire EDM as described herein, whereby the die-sink can be used to smoothen and thin the microwires in a final step. As an example, the microwires can be smoothed by using a die-sink EDM process, where a preformed block with holes is aligned to the grid and used to do the final forming step of the microwires. The die-sink described herein can be made of any material that is suitable for use as an EDM counter-electrode.

In some embodiments, the subtractive process to form the elongated protrusions 406 from the conductive block 405 may comprise an electrochemical machining (ECM) or micro-electrochemical machining process. ECM is generally used for working extremely hard materials, can be used to fabricate microelectrodes with high aspect ratio, and can be advantageous in some instances since there is no cutting force involved in material removal. ECM can be used to form structures similar to those made by die-sink or wire EDM, except the subtractive process in ECM is conducted within an electrochemical etching fluid. The electrochemical etching fluid can include a basic salt such as calcium chloride, an acid, or a base. ECM can be performed by passing an electrical current between a positively charged electrode (cathode), and a negatively charged electrode (anode). An electrolyte can be flown in between the electrodes. In ECM, the template tool 410 may be the cathode electrode (e.g. tool electrode) and the block 405 may be the anode electrode (e.g. workpiece electrode). In some embodiments, the ECM may comprise using low voltage and a high current (e.g. 5-30 V DC), and current densities ranging from about 10-500 A cm−2. The electrodes can be placed close to each other with a gap 416. The gap 416 may have a predetermined width, for example between about 80 um-800 um. In ECM, material from the anode or workpiece can be removed (via electro-chemical dissolution) as electrons cross the gap between the two electrodes. The tool electrode can be advanced towards the workpiece along one or more directions/axes, so as to form the workpiece into a desired shape. The electrolyte may carry the dissolved material. The electrolyte may include a conductive solution such as an aqueous solution of a salt (e.g. sodium chloride or sodium nitrate). In some embodiments, the electrolyte may be an acidic solution (e.g. HCl).

In some embodiments, the subtractive process to form the elongated protrusions 406 from the conductive block 405 may comprise an electrical discharge machining (EDM) process. EDM is also known by other names such as spark machining, spark eroding, die sinking, wire burning or wire erosion. In EDM, generally, a desired shape can be formed in a workpiece by removing materials using a series of rapidly recurring current discharges between the workpiece 450 and a counter electrode 410, which may be tool head or a tool template (e.g. a die or mold). In EDM, the workpiece 405 may be the negative electrode (anode) and the template 410 may be the positive electrode (cathode). Alternatively, in EDM the template 410 may be the negative electrode (anode) and the workpiece 405 may be the positive electrode (cathode). A dielectric fluid can be flown in between the two electrodes, such as the workpiece and the tool template. The dielectric fluid may be a non-conductive fluid. The dielectric fluid may temporarily conduct electricity under a predefined voltage being applied to the electrodes. In some embodiments, the dielectric fluid may include an oil (e.g. kerosene) or deionized water.

In EDM, the counter-electrode may apply a voltage below the spark formation voltage required on the workpiece to perform the machining. In some instances, the applied field may electrically dissolve the material on the workpiece locally against the counter-electrode. In contrast, ECM can allow for higher aspect ratio structures to be fabricated on the hermetic feedthrough device while improving smoothness of the resulting microwires. In some embodiments, an exemplary process may include first making gross/rough cuts using EDM or any other traditional bulk machining method, followed by a final finishing pass via ECM to achieve the desired final size or surface properties of the resulting microwires.

The foregoing description describes different subtractive processes that can be used to fabricate the microwires. In some other embodiments, additive manufacturing can be used to form a desired structure by adding material (instead of subtracting or removing material from a workpiece). In some embodiments, the elongated protrusions may be formed using additive manufacturing (e.g. 3D printing, laser sintering, shadow masking, local electrochemical deposition, and photolithography-based layer-by-layer (LBL) manufacturing). As an example, one or more additive manufacturing processes can be used to form electrodes/microwires directly on top of the feedthroughs on a feedthrough device/plate (instead of first bonding a block to the feedthroughs and machining the block using a subtractive process to form the electrodes/microwires). Forming the electrodes/microwires using an additive manufacturing process can provide the advantage of not requiring a solder or other bonding technique to pre-join the block to the feedthrough device/plate.

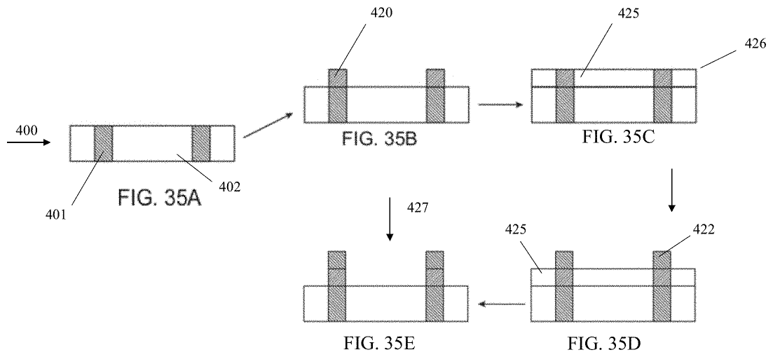

FIGS. 35A-35E illustrate an example of forming microwires using additive manufacturing, in accordance with some embodiments. The example shown in FIGS. 35A-35E may be a photolithography based layer-by-layer additive process. A photolithographic mask can be used to selectively deposit material over the feedthroughs. Due to the limited height of photolithographic processes, a number of layers may be necessary to achieve a final desired height. The conductive material may be formed by each layer, and a support material can be applied to provide support or structural reinforcement during the additive process. In some embodiments, a planarization step (e.g. chemical mechanical polishing) may be used to planarize one or more of the layers. The pattern definition process may include any lithography process including a mask-based etching process, electron beam, or laser. In some alternative embodiments, the layers can be formed using a shadow-masking process where the layers are deposited through a patterned barrier that is placed onto the substrate or offset from the substrate (instead of forming the layer directly on the substrate using photoresist). In some embodiments, each layer may be pre-defined on a support medium or laminate, such as on a tape, which is then applied to the surface of the substrate as a mask. The materials can be deposited using a variety of different deposition techniques, for example sintering, electroplating, reflow, or chemical or physical vapor deposition.

Referring to FIG. 35A, a substrate base 400 (e.g. feedthrough plate/device) comprising a plurality of electrical contacts (401 and 402) is provided. Next, as shown in FIG. 35B, a first material may be selectively deposited over the electrical contacts 401 and 402 of the substrate base 400 to form a first set of protrusions 420. The first set of protrusions 420 may be spaced apart by one or more gaps. Next, as shown in FIG. 35C, a support material 425 may be deposited in the gaps between and around the first set of protrusions 420. The support material 425 may provide structural support or reinforcement as the elongated protrusions are being formed. Next, as shown in FIG. 35D, a second material 422 may be selectively deposited over the first set of protrusions 420 to form a second set of protrusions. The layer by layer additive process may continue to sequentially add a plurality of protrusions stacked on top of one another, to form a plurality of high aspect ratio elongated protrusions. Due to the limited height of photolithographic processes, a plurality of layers may be required to achieve the final desired aspect ratio to form the elongated protrusion. The additive process may include sequentially stacking 2, 3, 4, 5, 6, 7, 8, 9, 10, 20, 30, 40, 50, 60, 70, 80, 90, 100, 200, 300, 400, 500 or more layers, or a number of layers between any two of the foregoing values, to form the elongated protrusions. The layers may have a same thickness or different thicknesses. In some embodiments, a first set of layers may have a first thickness and a second set of layers may have a second thickness that is different from the first thickness. The thickness of the any of the layers may range from about 1 um, 5 um, 10 um, 20 um, 30 um, 40 um, 50 um, 60 um, 70 um, 80 um, 90 um, 100 um, 200 um, 300 um, 400 um, 500 um or more, or a thickness between any two of the foregoing values.

The layers of the support material 425 may provide structural reinforcement to build up the elongated protrusions during the additive process. The support material 425 may be temporary and can be removed after the desired structures are formed, illustrated in FIG. 35E. The support material 425 may be etched using a preferential etching process that removes the support material 425 with little or no impact on the microwires and the substrate on which the microwires are supported. In some embodiments, the first material (first set of protrusions 420) may be the same as the second material (second set of protrusions 422). In some embodiments, the first material (first set of protrusions 420) may be different from the second material (second set of protrusions 422) to form heterogenous elongated protrusions comprising two or more different materials. In some embodiments, a portion of the elongated protrusions (e.g. microwires) may be formed from the first material, and another portion of the elongated protrusions may be formed from the second material. In some embodiments, a plurality of first portions of the elongated protrusions may be formed from the first material, and a plurality of second portions of the elongated protrusions may be made from the second material. The plurality of first portions and the plurality of second portions may be located at different sections of the elongated protrusions. In some embodiments, a plurality of layers of the first material may be deposited followed by a plurality of layers of the second material, or vice versa. In some embodiments, the first material may have a predefined set of properties that is different from the second material. The predefined set of properties may include physical properties, chemical properties, electrochemical properties, and/or electric conductivity. For example, the first material may be different from the second material to improve adhesion at the base of the elongated protrusions. In another embodiment, the second material may be stiffer than the first material to increase the stiffness at the tip of the elongated protrusions. An advantage of having a solid tip may be that in the event of electrode degradation, the electrochemical properties of the tip will remain relatively constant. In some embodiments, the first material may constitute the bulk of the microwire to provide stiffness/rigidity (e.g. the first material may comprise tungsten) along the length of the microwires. The second material may constitute the top, end or distal portion of the microwires, and may comprise a material that allows for enhanced neuronal recording (e.g. the second material may comprise platinum iridium).

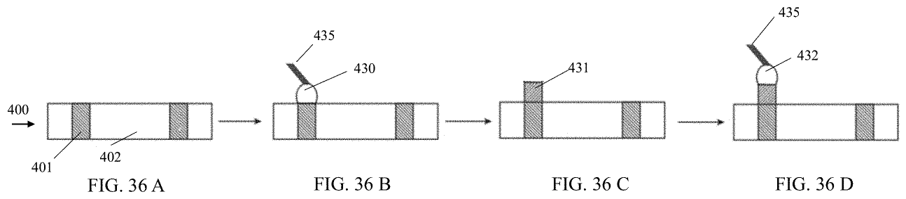

FIGS. 36A-36D show another example of a process for fabricating a microwire array using additive manufacturing, in accordance with some other embodiments. Elongated protrusions (e.g. microwires) may be directly formed on a substrate 400 (e.g. feedthrough plate/device) using local deposition. Local deposition can reduce or eliminate the need for a template, mask, or mold. Referring to FIG. 35A, a substrate base 400 (e.g. feedthrough plate/device) comprising a plurality of electrical contacts (401 and 402) is provided. Next, the local deposition may be delivered using a dispensing needle 435. The dispensing needle 435 may comprise one or more fluidic microchannels. FIG. 36B shows a first layer 430 being formed by depositing a droplet of material on the substrate (e.g. feedthrough). The droplet may then solidify or cure to form a first layer 431, as shown in FIG. 36C. Next, a second droplet 432 may be deposited adjacent or on top of the first layer 431 to form a second layer. An elongated protrusion (e.g. microwire) can be formed by sequential deposition of droplets adjacent or on top of one another. The term “adjacent” or “adjacent to,” as used herein, generally refers to ‘next to’, ‘adjoining’, ‘in contact with,’ or ‘in proximity to.’ “Adjacent to” may refer to one feature, such as a layer, being ‘above’, ‘beside’ or ‘below’ another feature, such as another layer. A first layer adjacent to a second layer may be in direct contact with the second layer, or there may be one or more intervening layers between the first layer and the second layer. The form of the structure may be determined by controlling and steering the dispensing needle. In some embodiments, the dispensing needle may be drawn in a direction substantially perpendicular to the surface of the substrate 400 (e.g. feedthrough plate) to form substantially vertical microwire arrays. The term “perpendicular” as used herein, generally refers to the angle between two features such as a microwire and the surface of a substrate (e.g. plate 400) to be within a range of about 85 degrees to 95 degrees. In some embodiments, the dispensing needle may have a voltage driven through it against the substrate 400 to deliver the droplets electrostatically and locally. In some cases, the resolution of the local deposition may be influenced in part by the diameter and movement resolution (e.g. along one or more axes) of the dispensing needle. For example, when printing electrodes on hermetic feedthroughs using the dispensing needle, the needle can be serially or sequentially placed over each feedthrough and drawn upwards, forming each electrode serially before moving onto the next one. A multilayer material can be deposited in the above fashion by changing the plating solution being delivered.

In some embodiments, a plurality of dispensing needles may be used to form a plurality of microwires. In some embodiments, the dispensing needles may deposit different materials to form microwires. In some embodiments, a first material and a second material may be deposited sequentially to form microwires. In some embodiments, the first material may be different from the second material to form heterogenous microwires. Alternatively, the first material may have a predefined set of properties that are different from the second material. In some embodiments, the predefined set of properties may include physical properties, chemical properties, electrochemical properties, and/or electric conductivity. For example, the first material may be different from the second material, and can be used to improve adhesion at the base of the elongated protrusions. In another embodiment, the second material may be stiffer than the first material, and may be used to increase the stiffness at the tip of the elongated protrusions. An advantage of having a solid tip may be that in the event of electrode degradation, the electrochemical properties of the tip can remain relatively constant. For example, the first material (e.g. tungsten) may constitute the bulk of the microwire and may provide stiffness/rigidity/flexibility along the length of the microwires. The second material (e.g. platinum iridium) may constitute the tips portion of the microwires and may comprise a material having a different electrochemical property that can allow for enhanced neuronal recording. In some embodiments, the dispense needle may include a position detector tool (e.g. an atomic force microscopy tip) to provide force feedback when the tip of the dispense needle contacts the substrate.

In some cases, the additive process may include laser sintering (e.g. selective laser sintering (SLS), selective laser melting (SLM), direct metal laser melting (DMLM), or laser powder bed fusion (LPBF)). In some cases, the laser sintering may include using a high power-density laser to melt and fuse metallic particles to form high aspect ratio structures (e.g. microwires). In some embodiments the feedthrough device may be immersed in a bath/medium containing metallic particles (e.g. bath of metal particles, powder bed), and a laser can be used to locally sinter or melt the metallic particles to form the desired structure (e.g. microwire) as the device is being drawn from the bath/medium.

The completion of an electric circuit between two or more electrodes can be utilized to form microwires, as described in one or more of the examples herein (e.g. ECM, EDM, electrochemical direct write, or electrochemical lithography). While the feedthrough pads on the feedthrough plate or the conductive block 405 are conductive, other portions of the feedthrough plate may be non-conductive or insulating. In some cases, electrical contact may not be easily formed between the electrodes since the feedthrough plate is insulating. To overcome the above challenge, the present disclosure provides techniques that can be used to form complex or high aspect ratio structures on either each individual feedthrough or on small sub-regions comprised of a smaller number of feedthroughs than the entire plate. Accordingly, each of the above structures can be electrically isolated from one another (alternatively, the structures need not be electrically isolated if all of the feedthroughs are bonded to a single electrode). Using the techniques disclosed herein can allow proper establishing of electrical contact in order to complete the circuit through the feedthrough plate.

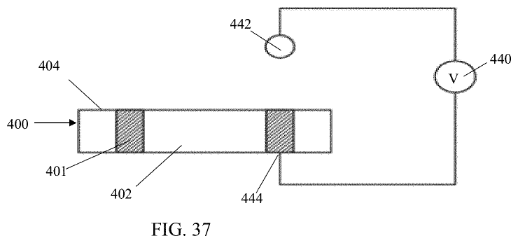

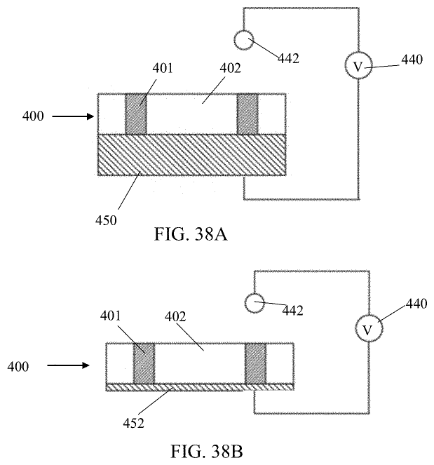

In some embodiments, an electrode may comprise of a single individual feedthrough conductive pad (e.g., conductive feedthrough). In other cases, an electrode may be formed by joining a plurality of the conductive pads. In some embodiments, all of the feedthrough conductive pads may be electrically joined to form an electrode. FIG. 37 illustrates an example of an electrical circuit including a first electrode 442 (e.g. EDM or ECM template tool), a source of electrical potential 440, and a second electrode 444 (e.g. a conductive pad 401 on a feedthrough plate 400). FIGS. 38A and 38B show examples of connecting multiple feedthrough conductive pads 401 to form an electrode. The circuits in FIGS. 38A and 38B comprise a first electrode 442, a source of electrical potential 440, and a second electrode 450 and 452, respectively. In some embodiments, the second electrode may be electrically connected to a plurality of feedthrough conductive pads 401. In some embodiments the feedthroughs may be electrically connected by bonding a conductive block 450 on the backside of the feedthrough plate 400, as shown in FIG. 38A. In some embodiments, the conductive block 450 bonded to the backside of the feedthrough plate 400 may include materials different from another conductive block bonded to the front side of the feedthrough plate 400. The conductive block 450 may comprise materials that are chemically dissimilar to the electrodes on the frontside of the feedthrough plate 400. The conductive block 450 may be used to form an electrical connection between two or more feedthroughs. In some embodiments, the two or more feedthroughs may include some but not all of the feedthroughs in the feedthrough plate. In some embodiments the two or more feedthroughs may include all the feedthroughs in the feedthrough plate. In some embodiments, the conductive block 450 may be removed after microwires are formed using physical methods (e.g. machining, grinding), chemical methods (etching), or combined methods (e.g. plasma etching).

FIG. 38B shows another example of connecting feedthroughs electrically using a thin layer, in accordance with some embodiments. The thin layer 452 may be a thin film of conductive material (e.g. metal, metallic alloy, graphite). The thin layer 452 can be formed as an electrical bridge on the backside of the feedthrough plate. In some embodiments, the thin film may have a predefined thickness to enable electrical current to pass through. The predefined thickness may be between about 100 nm to 100 um. In some embodiments, the thickness may be at least about 50 nm, 75 nm, 100 nm, 150 nm, 175 nm, 200 nm, 500 nm, 750 nm, 1 um, 10 um, 20 um, 50 um, 75 um, 100 um, 200 um, or more. In some embodiments, the thickness may be at most about 200 um, 100 um, 75 um, 50 um, 20 um, 10 um, 1 um, 750 nm, 500 nm, 200 nm, 175 nm, 150 nm, 100 nm, 75 nm, 50 nm, or less.

In some embodiments, a thin film of conductive material 452 may be applied to the backside of the feedthrough plate 400 using chemical vapor deposition (CVD) technology. CVD technology may include atmospheric pressure chemical vapor deposition (APCVD), low pressure chemical vapor deposition (LPCVD), plasma assisted (enhanced) chemical vapor deposition (PACVD, PECVD), photochemical vapor deposition (PCVD), laser chemical vapor deposition (LCVD), metal-organic chemical vapor deposition (MOCVD) and chemical beam epitaxy (CBE).

In some embodiments, a thin film of the conductive material 452 may be applied to the backside of the feedthrough plate 400 using physical vapor deposition (PVD) technology. PVD technology may include sputtering, ion plasma assist, thermal evaporation, vacuum evaporation, and molecular beam epitaxy (MBE). In some embodiments the thin film of a conductive material 452 may be applied to the backside of the feedthrough plate 400 using solution bath plating technology (SBP). Solution bath plating may include electroplating, electroless plating, or electrolytic plating technology. In some embodiments, a first layer coating may be required to prepare nonmetallic surfaces (e.g. plastic, or glass) on the backside of the feedthrough plate 400 for solution bath plating. The first layer coating may include chromium, nickel, aluminum, tin, tin-bismuth alloy, gold, or gold-tin alloys. In some embodiments the thin film of a conductive material 452 may be applied to the feedthrough plate 400 using cold-gas dynamic spray (e.g. cold spray). Cold spray process may include spraying of powdered metals, alloys, or mixtures of metal and alloys onto an article using a jet of high velocity gas to form continuous metallic coating at temperatures below the fusing temperatures of the powdered material (e.g. aluminum). In some embodiments, a first layer coating (e.g. tin, zinc, silver or gold) may be applied to the backside of the feedthrough plate 400 to pretreat the nonmetallic surfaces (e.g. glass, or plastic) for metallic coating using cold spray.

In some embodiments, a thicker film of the conductive material 452 may be formed using metal reflow. In some embodiments, metal reflow may include solder reflow. A plurality of solder bumps may be formed on the backside of the feedthrough plate. The solder bumps may be formed of any type of binary or ternary solder alloys. In some cases, the solder bumps may be formed of a lead-free solder such as SnAg, or a SnAg alloy (e.g. SnAgCu). In some instances, the solder bumps may be formed of a low melting point metal or metallic alloy (e.g. Indium, or an Indium alloy). In some cases, solder balls may be physically placed onto the conductive pads (or conductive feedthroughs) of the feedthrough plate and reflowed.

In some embodiments, the feedthroughs can be electrically joined to form the workpiece electrode by connecting a control circuit to the feedthrough plate. In some embodiments, the solder bumps may be formed on the back side of the feedthrough plate, and the solder bumps may then be joined (connected) to a conductive plane or the control circuit via flip-chip bonding before electrode formation. In some embodiments, the bond may be temporary and may be disconnected after the microwires are formed. For example, a solder bond can be broken by etching away the solder away using a solution that can dissolve the solder without affecting the electrodes. In some embodiments, instead of forming a full bond, the solder bumps can be mechanically pressed to the circuit so as to form a temporary bond via cold pressing. In some embodiments, the circuit may comprise a control circuit (e.g. printed circuit board (PCB), a ceramic plane, or an integrated circuit). The circuit may be modified to form a fanout connector for channel selection. In some embodiments, all of the channels in a circuit can be unified to form a single electrode or counter-electrode. In some embodiments, a plurality of channels but not all channels in a circuit may be unified to form a single electrode or counter-electrode. In some embodiments, the circuit can be modified to include active elements for assisting with the deposition process. Those active elements may include current control or voltage control for individual channels. The active elements can also be used for analysis of the deposition process as the process is occurring via any given channel. Using active elements in a custom circuit to form the workpiece electrodes may have advantages including having control over the formation of the protrusions. For example, forming heterogenous microwires (e.g. in a parallel process such as patterned electroplating) may be controlled on an individual feedthrough level instead of relying on a uniform deposition.

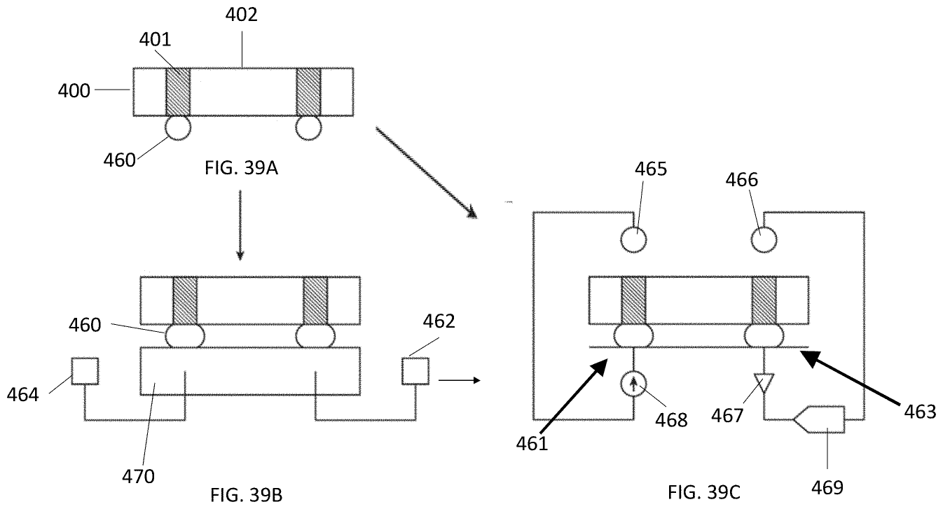

FIG. 39A-39C show an example of connecting a control circuit to the feedthrough plate. FIG. 39A shows the feedthrough plate 400 comprising the feedthrough conductive pads 401, the insulating portions 402, and solder bumps 460 formed on the backside of the feedthrough plate. FIG. 39B shows a control circuit (e.g. PCB, ceramic plane, or integrated circuit) being connected to the feedthrough plate 400 via the solder bumps 460 on the backside of the feedthrough plate. In some embodiments, solder reflow may be also used to bond the feedthrough plate to the control circuit. In some cases, the fixture 470 may be a fanout connector, unifying a plurality of lines into a single electrode. Alternatively, each of the feedthroughs may be independently controlled via an individual channel (e.g. 462 and 464). FIG. 39C shows an example of controlling the deposition process by using active elements in the circuit. In some embodiments, controlling the deposition via active elements may require a plurality of active elements. In some embodiments, the plurality of active elements may include a current controller 468 and/or a voltage controller 467. The current controller 468 may form a circuit 461 with an electrode 465. The electrode 465 may a tool electrode (e.g. ECM template tool, EDM template tool). Another electrode within circuit 461 may be the workpiece electrode (e.g. a feedthrough, an array of feedthroughs, a microwire, or an array of microwires). The voltage controller 467 may form a circuit 463 with an electrode 466. Similarly, the electrode 466 may be the tool electrode (e.g. ECM template tool, EDM template tool). Another electrode within circuit 463 may be the workpiece electrode (e.g. a feedthrough, an array of feedthroughs, a microwire, or an array of microwires). Accordingly, the active elements shown in FIG. 39C can allow for either current control and/or voltage control over individual channels to control or modulate the deposition process.

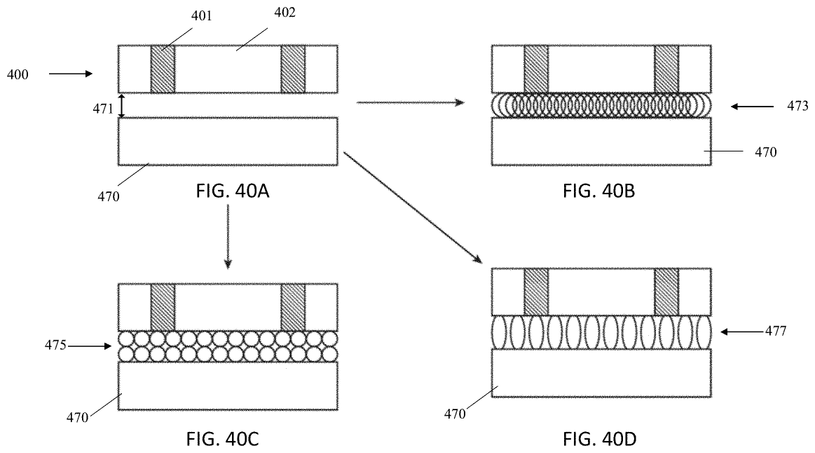

As described above, the circuit board may be attached to the feedthrough plate using solder bumps. This can allow for control over individual feedthroughs. In some other embodiments, conductive material can be applied to the gap 471 between the circuit 470 and the backside of the feedthrough plate to make an electrical bridge between a circuit and a plurality of feedthroughs. In some embodiments, the electrical bridge may be temporary and may comprise conductive ink 473, conductive adhesive/tape 475, and/or an anisotropic conductor layer 477, as illustrated in FIGS. 40B, 40C, and 40D respectively. Similarly, a conductive block similar to block 450 or a thin film similar to layer 452 may be bonded to the feedthrough plate via the aforementioned temporary bonding techniques (e.g. using conductive ink, conductive tape, or anisotropic conductor).

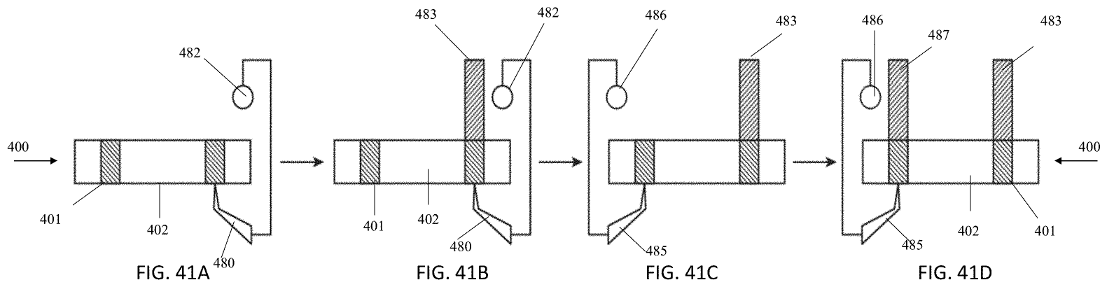

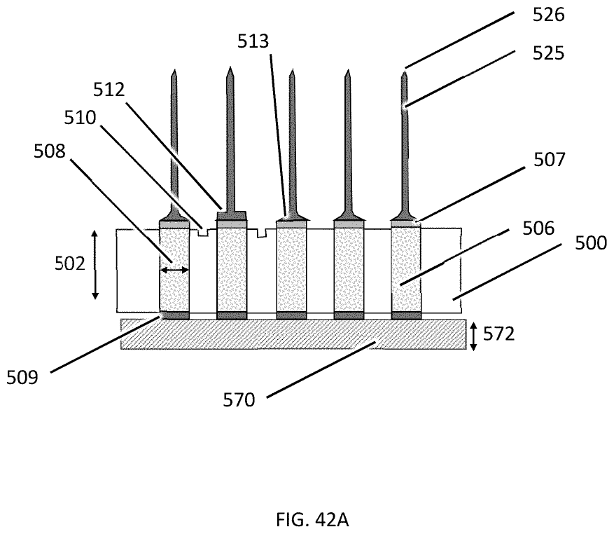

FIGS. 41A to 41D show examples of connecting individual feedthrough conductive pads to form the workpiece electrode to control the process of forming the microwires. In some embodiments, a physical electrical contact may be temporarily provided on the backside of the feedthrough plate 400. The contact may be made using an individual probe 480 or an array of probes. The probe can make contact at a first conductive pad on the backside of the feedthrough plate 400 and may be connected to a second electrode 482 (e.g. template tool). An electrical potential source, similar to 440 described elsewhere herein, may be connected in between the electrodes. A first high aspect protrusion 483 or an array of high aspect ratio protrusions may be formed by any of the techniques (e.g. ECM, EDM or electrolithography) described herein using the electrodes, for example as shown in FIG. 41B. Referring to FIG. 41C, a second probe 485 may be placed to connect a third electrode 486 to a conductive pad 401 or an array of pads on the backside of the feedthrough plate 400. Similarly, a second high aspect ratio protrusion 487 or an array of high aspect ratio protrusions may be formed using the electrodes. The above shows an example of controlling formation of microwires on an individual feedthrough level. In some embodiments, a similar method can be used to form heterogenous microwires on an individual feedthrough level. It should be noted that the probes can match whichever side (front side of backside of the feedthroughs) that is being built up or machined, and that the probes can be connected to control and test circuitry as well.

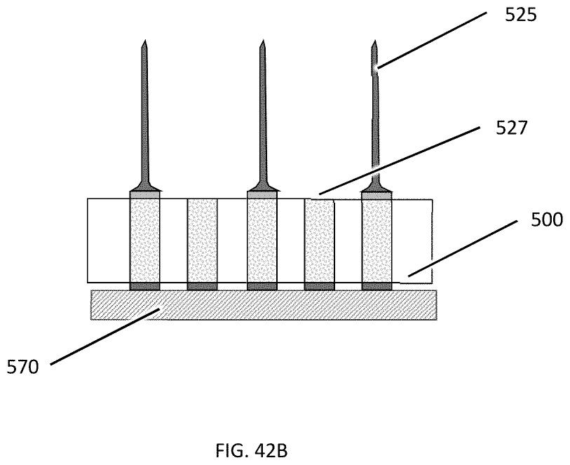

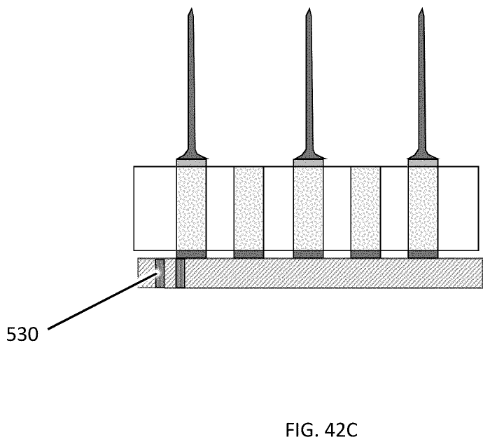

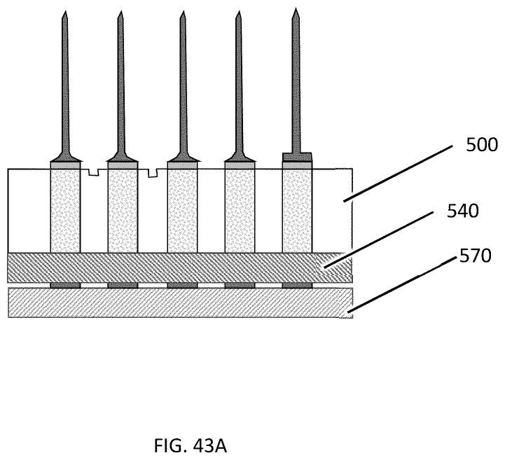

FIG. 42A illustrates an example of a neural interface probe device comprising a plurality of microwires 525, substrate 500, and a chip 570. The substrate 500 may comprise a plurality of conductive feedthroughs 506. The substrate 500 may be a ceramic substrate. Alternatively, the substrate may comprise, or may be made of any of the substrate materials as described elsewhere herein. The substrate 500 may have a thickness 502 of at most about 1 millimeter (mm). The thickness 502 of the substrate may be from about 100 micrometers (μm) to about 1 mm, from about 200 μm to about 900 μm, from about 300 μm to about 800 μm, from about 400 μm to about 700 μm, or about 500 μm to about 1 mm. The substrate 500 may comprise cut sections 510 in between the microwires 525. The cut sections may render the substrate surface to be non-smooth. The cut sections may be formed as a result of forming the microwires using any of the methods described herein.

The plurality of microwires 525 may be made from a material comprising a noble metal. The plurality of microwires 525 may each comprise a sharpened tip 526. A distal end of a microwire (e.g., an end farther from a feedthrough substrate) may be modified to form a conical tip. The sharpened tip 526 may have a conical radius. The plurality of conductive feedthroughs 506 may include a material comprising a metal, cermet, or a combination thereof. Cermet may comprise a heat-resistant material comprising ceramic and metal (e.g., sintered metal). In some embodiments, the material of the plurality of conductive feedthroughs 506 may be similar to, or may be different from a material of the plurality of microwires 525. A base of a microwire 525 (e.g., an end closer to the substrate 500) may have a square planar shape 512 or fillet shape 513. In some embodiment, the base of a microwire may be formed into a fillet to avoid sharp edges that may provide better accessibility for coating.

The plurality of microwires 525 may have a thickness or diameter that monotonically reduces from a thicker portion to a thinner portion. The thicker portion may be closer to the substrate 500. The thinner portion may be closer to the tip 526. In some embodiment, a conductive block may be subtracted such that each microwire in the array of microwires may have a diameter that decreases monotonically from a proximal end to a distal end of the microwire. Each microwire may have an average thickness or diameter from about 10 μm to about 50 μm. The thickness or diameter may be from about 15 μm to about 45 μm, from about 20 μm to about 40 μm, or from about 25 μm to about 35 μm.

The conical radius of the sharpened tip 526 may be between about 1 micrometer (μm) to about 10 μm wide. In some embodiment, the sharpened tip may have a conical radius of about 1 μm to about 2 μm, about 1 μm to about 3 μm, about 1 μm to about 4 μm, about 1 μm to about 5 μm, about 1 μm to about 6 μm, about 1 μm to about 7 μm, about 1 μm to about 8 μm, about 1 μm to about 9 μm, about 1 μm to about 10 μm, about 2 μm to about 3 μm, about 2 μm to about 4 μm, about 2 μm to about 5 μm, about 2 μm to about 6 μm, about 2 μm to about 7 μm, about 2 μm to about 8 μm, about 2 μm to about 9 μm, about 2 μm to about 10 μm, about 3 μm to about 4 μm, about 3 μm to about 5 μm, about 3 μm to about 6 μm, about 3 μm to about 7 μm, about 3 μm to about 8 μm, about 3 μm to about 9 μm, about 3 μm to about 10 μm, about 4 μm to about 5 μm, about 4 μm to about 6 μm, about 4 μm to about 7 μm, about 4 μm to about 8 μm, about 4 μm to about 9 μm, about 4 μm to about 10 μm, about 5 μm to about 6 μm, about 5 μm to about 7 μm, about 5 μm to about 8 μm, about 5 μm to about 9 μm, about 5 μm to about 10 μm, about 6 μm to about 7 μm, about 6 μm to about 8 μm, about 6 μm to about 9 μm, about 6 μm to about 10 μm, about 7 μm to about 8 μm, about 7 μm to about 9 μm, about 7 μm to about 10 μm, about 8 μm to about 9 μm, about 8 μm to about 10 μm, or about 9 μm to about 10 μm. In some embodiment, the sharpened tip (e.g., conical tip) may have a conical radius of about 1 μm, about 2 μm, about 3 μm, about 4 μm, about 5 μm, about 6 μm, about 7 μm, about 8 μm, about 9 μm, or about 10 μm. In some embodiment, the sharpened tip (e.g., conical tip) may have a conical radius of at least about 1 μm, about 2 μm, about 3 μm, about 4 μm, about 5 μm, about 6 μm, about 7 μm, about 8 μm, about 9 μm, or more. In some embodiment, the sharpened tip (e.g., conical tip) may have a conical radius of at most about 10 μm, about 9 μm, about 8 μm, about 7 μm, about 6 μm, about 5 μm, about 4 μm, about 3 μm, about 2 μm, about 1 μm, or less.

The plurality of conductive feedthroughs 506 may have a width or diameter 508. The width 508 may be from about 25 μm to about 250 μm. The width 508 may be from about 50 μm to about 225 μm, from about 100 μm to about 200 μm, or from about 125 μm to about 175 μm.

A microwire 525 may be connected to a conductive feedthrough 506 using solder or braze. The microwire may be formed using any of the techniques described herein. The solder or braze connection 507 may comprise a biocompatible material (e.g., gold or gold-titanium alloy). The solder or braze connection 507 may have a thickness of at most about 200 micrometers (μm). The connection 507 can connect a microwire 525 to a conductive feedthrough 506 in the feedthroughs without causing any electrical shorting between adjacent microwires. In some embodiments, the solder or braze connection 507 may have a thickness of about 1 μm to about 5 μm, about 1 μm to about 20 μm, about 1 μm to about 50 μm, about 1 μm to about 100 μm, about 1 μm to about 150 μm, about 1 μm to about 200 μm, about 5 μm to about 20 μm, about 5 μm to about 50 μm, about 5 μm to about 100 μm, about 5 μm to about 150 μm, about 5 μm to about 200 μm, about 20 μm to about 50 μm, about 20 μm to about 100 μm, about 20 μm to about 150 μm, about 20 μm to about 200 μm, about 50 μm to about 100 μm, about 50 μm to about 150 μm, about 50 μm to about 200 μm, about 100 μm to about 150 μm, about 100 μm to about 200 μm, or about 150 μm to about 200 μm. In some embodiments, the solder or braze connection 507 may have a thickness of about 1 μm, about 5 μm, about 20 μm, about 50 μm, about 100 μm, about 150 μm, or about 200 μm. In some embodiments, the solder or braze connection 507 may have a thickness of at least about 1 μm, about 5 μm, about 20 μm, about 50 μm, about 100 μm, about 150 μm, or about 200 μm. In some embodiments, the solder or braze connection 507 may have a thickness of at most about 200 μm, about 150 μm, about 100 μm, about 50 μm, about 20 μm, about 10 μm, about 5 μm, about 1 μm, or less.

The chip 570 may be an integrated circuit (IC) chip. The integrated circuit chip may be an application-specific integrated circuit (ASIC). The chip may have a thickness 572. The thickness 572 may be at most as thick as the substrate thickness 502. In some cases, the thicknesses 572 and 502 can be substantially the same. In other cases, the chip can be thicker than the substrate.

The electrical chip and the substrate (e.g., feedthrough device) may be connected using solder or braze via connection 509. The connection 509 may comprise a different material relative to the connection 507. The material of the connection 509 may comprise non-biocompatible material. In some embodiments, the material of the connection 509 may comprise biocompatible material.

FIG. 42B illustrates another example of a neural interface probe device comprising a plurality of microwires 525, a plurality of non-wired conductive feedthroughs 527, substrate 500, and a chip 570. As shown in FIG. 42B, the non-wired conductive feedthroughs 527 need not be connected to any microwire. In some embodiments, different length microwires may be used to allow for collecting signal from various depth of a sample (e.g., a human subject, an animal, etc.) The non-wired conductive feedthroughs may be used to collect (or record) signals from the surface as compared to microwires recording signals at a depth farther from a surface of a sample (e.g., brain tissue of a human subject or an animal). For example, in FIG. 42A the device may record from microwires that can penetrate into a brain to primarily record single unit neurons and/or local field potential signals. In another example, in the device schematically shown in FIG. 42B, microwires may record from the depth and non-wired feedthroughs may collect signals from regions closer to the surface of the brain. In some embodiments, the signals that can be collected by a mix of microwires and non-wired feedthroughs may comprise high-density or intermixed.

FIG. 42C illustrates another example of a neural interface probe device, in which the chip comprises vertical interconnect access (via) 530. The via 530 may be for example a through silicon via (TSV). In some embodiments, a TSV may be used to integrate additional components on the chip. In some embodiments, the TSV may be placed on the chip using drilling. The TSV may be placed on the chip using other bonding or connecting methods. In some embodiments, TSV allows for manufacturing more compact device (e.g., less volume or narrower in width or length). The TSV may be used as a vertical electrical connection that passes through the thickness of the chip. In some embodiments, the TSV may allow for higher rate of data transfer. In some embodiments the TSV may be used to transfer data directly from the feedthrough device to another device or bypass a chip. In some embodiments, the TSV may be used to allow electrical connection between two or more chips. The two or more chips may form a three-dimensional (3D) integrated circuit.

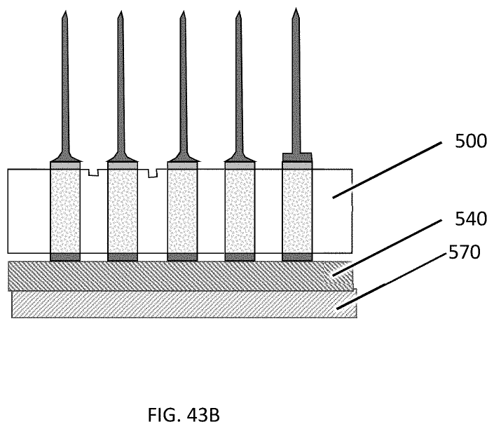

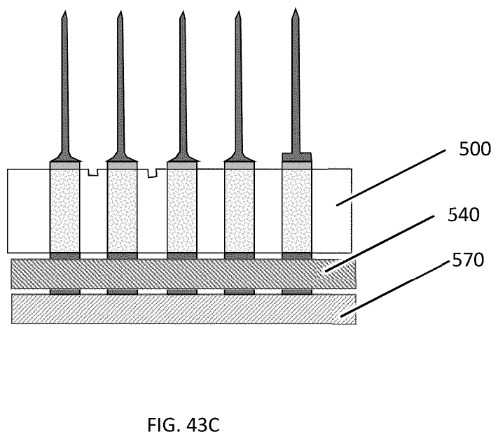

FIGS. 43A-43C illustrate examples of neural interface probe devices comprising a redistribution layer 540. Referring to FIG. 43A, the redistribution layer (RDL) 540 may be attached to the substrate 500 (e.g., to the feedthroughs). The RDl and the feedthrough substrate may be pre-fabricated to be attached. The redistribution layer may be subsequently connected to the chip 570 using solder or braze. Referring to FIG. 43B, the redistribution layer 540 may be attached to the chip 570 (e.g., on chip); the RDL and the chip may be attached prior to use as described herein. In some embodiments, the RDL may be bonded to the chip or the substrate using a method sufficient to bond the RDL and the chip or the feedthrough substrate (e.g., diffusion bonding, conductive ink, conductive tape, other adhesives, etc.) The substrate (or feedthrough) may be subsequently connected to the redistribution layer using solder or braze connections. Referring to FIG. 43C, the redistribution layer 540 may be a separate component that can be individually connected to the substrate 500 and/or chip 570 using solder or braze connections. The solder or braze connections may comprise a biocompatible material. In other cases, the solder or braze connections may comprise a non-biocompatible material. In some embodiments, the solder or braze connections may comprise a biocompatible material (e.g. between the substrate and the redistribution layer) and a non-biocompatible material (e.g. between the redistribution layer and the chip).

A redistribution layer (RDL) may comprise a metal or a metal alloy. In some embodiments, the RDL may be used on a chip (FIG. 43B) to redistribute or redirect electrical connections (e.g., input/output pins or IO pads) of a chip. For example, to make the IO pads available in other locations. In some embodiments, the RDL may be attached to a feedthrough substrate (or feedthrough device) (FIG. 43A) to redistribute or redirect electrical connections (e.g., input/output pins or IO pads) of a feedthrough. RDL may be used to match the IO pins (or pads) of a feedthrough and the IO pins (or pads) of a chip so that the data can be transferred between the feedthrough and the chip. The RDL may be used to facilitate feedthrough-to-chip bonding.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed.

What is claimed is:

1. An implantable device comprising:
a substrate comprising a plurality of feedthroughs, wherein the plurality of feedthroughs comprises a first conductive material; and
an array of microwires extending from the substrate, wherein the array of microwires is connected or bonded to the plurality of feedthroughs using a biocompatible solder or braze material or intermediate filler material, and wherein the array of microwires comprises a second conductive material that is different from the first conductive material.

2. The device of claim 1, wherein the substrate comprises ceramic.

3. The device of claim 1, wherein a thickness of the substrate is equal to or less than about 1 millimeter (mm).

4. The device of claim 1, wherein a diameter of each of the plurality of feedthroughs is from about 25 microns to about 250 microns.

5. The device of claim 1, wherein the plurality of feedthroughs is completely filled with the first conductive material.

6. The device of claim 1, wherein sidewalls of the plurality of feedthroughs are coated with the first conductive material.

7. The device of claim 1, wherein each microwire in the array of microwires has a conical tip.

8. The device of claim 7, wherein a radius of the conical tip is less than about 5 micrometers.

9. The device of claim 1, wherein each microwire in the array of microwires has a diameter of about 10 micrometers to about 50 micrometers.

10. The device of claim 1, wherein each microwire in the array of microwires has a diameter that decreases monotonically from a proximal end to a distal end of the microwire.

11. The device of claim 10, wherein the proximal end of the microwire is located closer to the substrate than the distal end.

12. The device of claim 11, wherein the proximal end of the microwire has a flange that is at least about 50% of a diameter of the feedthrough on which the microwire is located.

13. The device of claim 11, wherein a diameter of the distal end of the microwire is less than about 50% of a diameter of the feedthrough on which the microwire is located.

14. The device of claim 1, wherein a thickness of the biocompatible solder or braze material is less than about 200 micrometers.

15. The device of claim 14, wherein the biocompatible solder or braze material is configured to connect the array of microwires to the plurality of feedthroughs without causing electrical shorting between adjacent feedthroughs.

16. The device of claim 1, further comprising a ceramic film coated over or onto the array of microwires.

17. The device of claim 16, wherein a thickness of the ceramic film is about 500 nanometers to about 2 micrometers.

18. The device of claim 16, wherein the ceramic film is further coated on one side of the substrate.

19. The device of claim 1, wherein each microwire in the array of microwires has a de-insulated tip.

20. The device of claim 19, wherein the de-insulated tip has an impedance of about 50 kilo-ohms to about 5000 kilo-ohms when tested at a frequency of about 1 KHz in a biological saline solution.

21. The device of claim 1, wherein the array of microwires is configured to be inserted into brain tissue.
